(19) United States
(12) Patent Application Publication
(10) Pub. No.: US 2026/0232844 A1
(43) Pub. Date: Aug. 13, 2026

(54) GENE EDITING METHOD FOR INHIBITING ABERRANT SPLICING IN STATHMIN 2 (STMN2) TRANSCRIPT

(71) Applicant: ARBOR BIOTECHNOLOGIES, INC., Cambridge, MA (US)

(72) Inventors: Ryan Phillip ABO, Cambridge, MA (US); Chee Yeun CHUNG, Cambridge, MA (US); Anthony James GARRITY, Cambridge, MA (US); Priscilla DAVIDSON NEGRAES, Cambridge, MA (US)

(21) Appl. No.: 19/156,631

(22) PCT Filed: Feb. 15, 2024
(86) PCT No.: PCT/US2024/015940

§ 371 Date: Aug. 14, 2025

Related U.S. Application Data
(60) Provisional application No. 63/445,926, filed on Feb. 15, 2023.
(60) Provisional application No. 63/618,201, filed on Jan. 5, 2024.
(60) Provisional application No. 63/549,007, filed on Feb. 2, 2024.

(51) Int. Cl. A61K 48/00; A61K 9/00; A61K 38/17; A61P 25/28; C12N 9/22; C12N 15/11; C12N 15/86

(52) U.S. Cl. CPC A61K 48/0058; A61K 9/0085; A61K 38/1709; A61K 48/0075; A61P 25/28; C12N 9/226; C12N 15/111; C12N 15/86; C12N 2310/20; C12N 2750/14143

(57) ABSTRACT

A method for inhibiting aberrant splicing in a Stathmin-2 (STMN2) transcript, the method comprising: genetically editing a STMN2 gene in a cell to delete (a) one or more nucleotides in a 3′ splice site of intron 1, wherein the 3′ splice site is adjacent to exon 2a, (b) one or more nucleotides in a region of intron 1 that is adjacent to the 3′ splice site, or both (a) and (b), thereby inhibiting production of STMN2 transcripts including exon 2a and improving production of functional STMN2 transcripts in the cell. Also provided herein are gene editing systems for genetic modification of the STMN2 gene.

### CROSS REFERENCE TO RELATED APPLICATIONS

This application claims the benefit under 35 U.S.C. § 119 (e) of U.S. Provisional Application No. 63/549,007, filed Feb. 2, 2024, U.S. Provisional Application No. 63/618,201, filed Jan. 5, 2024, and U.S. Provisional Application No. 63/445,926, filed Feb. 15, 2023, the contents of each of which are incorporated by reference herein in their entirety.

### SEQUENCE LISTING

The instant application contains a Sequence Listing, which has been filed electronically in XML format and is hereby incorporated by reference in its entirety. Said XML copy, created on Feb. 13, 2024, is named 063586-511001WO_SeqList_ST26.xml and is 1,675,184 bytes in size.

### BACKGROUND OF THE INVENTION

Amyotrophic lateral sclerosis (ALS) is a late-onset neurodegenerative disorder characterized by progressive loss of motor neurons in the spinal cord and brain. The majority of the cases is sporadic and, among the 10% due to gene mutations, the GGGGCC repeat expansions in the chromosome 9 open reading frame 72 (C9orf72) represents the most frequent genetic cause of ALS. Many genes compromised in ALS are also involved with frontotemporal dementia (FTD), another neurodegenerative disease.

TDP-43 (transactive response DNA-binding protein 43) cytoplasmic accumulation is observed in both ALS and FTD. TDP-43 is a DNA/RNA binding protein implicated in RNA transcription, splicing, processing, transport, and stability. TDP-43 binds to thousands of pre-mRNA targets and its reduction in adult nervous system impacts the splicing or expression of many RNAs. Recently, it was noticed that stathmin-2 mRNA is significantly lost after TDP-43 depletion.

Stathmin-2 gene (STMN2) encodes a microtubule binding protein, crucial for maintaining axonal health. STMN2 is aberrantly spliced in ALS due to TDP-43 loss of function, generating a nonfunctional truncated form of STMN2 (exon 2a variant). Approaches to inhibit such STMN2 aberrant splicing and enhancing the production of functional STMN2 transcripts/proteins would benefit the treatment of diseases such as ALS and FTD.

### SUMMARY OF THE INVENTION

The present disclosure is based, at least in part, on the discoveries that gene editing at specific locations (e.g., a 3′ splice site in intron 1 and/or a region in intron 1 that is adjacent to the 3′ splice site) in the STMN2 gene significantly reduced aberrant splicing of STMN2 (reduced formation of the non-functional transcript containing the cryptic exon 2a) and increased production of full-length STMN2 transcripts. Accordingly, provided herein are methods and gene editing systems for inhibiting aberrant splicing in the STMN2 transcripts. Such methods and gene editing systems can benefit treatment of diseases involving loss of STMN2, for example, ALS and FTD.

In some aspects, the present disclosure provides a gene editing system, comprising: (a) a Type V nuclease (e.g., those disclosed herein such as SEQ ID NO: 3, 4, 5, or 6, or a variant thereof as also disclosed herein) or a nucleic acid encoding the nuclease; and (b) one or more guide RNAs (gRNAs) targeting the STMN2 gene, or one or more nucleic acids encoding the one or more gRNAs. The gene editing system leads to (a) a deletion of one or more nucleotides in a 3′ splice site of intron 1 of STMN2, wherein the 3′ splice site is adjacent to exon 2a; (b) a deletion of one or more nucleotides in a region of intron 1 that is adjacent to the 3′ splice site, or both (a) and (b), thereby reducing production of STMN2 transcripts including exon 2a and increasing production of functional STMN2 transcripts in a cell edited by the gene editing system.

In some instances, the gene editing system comprises two or more gRNAs targeting the STMN2 gene, for example, a first gRNA and a second gRNA. In some examples, the first gRNA induces a deletion of one or more nucleotides in the 3′ splice site and the second gRNA induces a deletion of one or more nucleotides in the downstream 10-nt window. In other examples, the first gRNA induces a deletion in both the 3′ splice site and the downstream 10-nt window and the second gRNA induces a deletion in the 3′ splice site. In yet other examples, the first gRNA induces a deletion in both the 3′ splice site and the downstream 10-nt window and the second gRNA induces a deletion in the downstream 10-nt window. In still other examples, each of the first gRNA and the second gRNA induces a deletion in both the 3′ splice site and the downstream 10-nt window.

In some examples, the gene editing system disclosed herein comprises (a) a Cas12i2 nuclease, which optionally comprises an amino acid sequence at least 90% identical to SEQ ID NO: 3 or SEQ ID NO: 266; and (b) the one or more guide RNAs (gRNAs) are selected from those listed in Table 2. In specific examples, the gene editing system comprises the nuclease of SEQ ID NO:3 or SEQ ID NO: 266 and a gRNA of G53, G55, or G56.

In some examples, the gene editing system disclosed herein comprises (a) a Type V nuclease comprising an amino acid sequence at least 90% identical to SEQ ID NO: 4 or SEQ ID NO: 255; and (b) the one or more guide RNAs (gRNAs) are selected from those listed in Table 3. In specific examples, the gene editing system comprises the nuclease of SEQ ID NO: 4 or SEQ ID NO: 255 and a gRNA of A_STMN2_Splice2a_4 or A_STMN2_Splice2a_4. In one example, the gRNA is A_STMN2_Splice2a_4. In another example, the gRNA is A_STMN2_Splice2a_3.

In some examples, the gene editing system disclosed herein comprises (a) a Type V nuclease comprising an amino acid sequence at least 90% identical to SEQ ID NO: 5; and (b) the one or more guide RNAs (gRNAs) are selected from those listed in Table 4.

In some examples, the gene editing system disclosed herein comprises (a) a Type V nuclease comprising an amino acid sequence at least 90% identical to SEQ ID NO: 6; and (b) the one or more guide RNAs (gRNAs) are selected from those listed in Table 5.

In some embodiments, the gene editing system disclosed herein comprises a nucleic acid encoding the Type V CRISPR nuclease. In some examples, the nucleic acid is a vector, which comprises a nucleotide sequence encoding the Type V CRISPR nuclease. The coding nucleotide sequence can be in operable linkage to a promoter. In some examples, the vector can be an adeno-associated viral (AAV) vector (e.g., an AAVrh10 vector). In some examples, the promoter can be a synapsin 1 promoter.

In addition, the present disclosure features a method for inhibiting aberrant splicing in a Stathmin-2 (STMN2) transcript, the method comprising: (i) genetically editing a STMN2 gene in a cell to delete (a) one or more nucleotides in a 3′ splice site of intron 1, wherein the 3′ splice site is adjacent to exon 2a, (b) one or more nucleotides in a region of intron 1 that is adjacent to the 3′ splice site, or both (a) and (b), thereby inhibiting production of STMN2 transcripts including exon 2a and improving production of functional STMN2 transcripts in the cell. In some embodiments, the method may further comprise (ii) measuring levels of STMN2 transcripts and/or STMN2 protein in the cell after the genetic editing.

In some embodiments, the 3′ splice site in (a) comprises the nucleotide sequence of TTGCAG. Alternatively or in addition, the region of exon 2a in (b) comprises the nucleotide sequence of ACTCGGCAGA (SEQ ID NO: 2) (also referred herein as the downstream 10-nt window).

In some embodiments, the gene editing step (i) may result in deletions of one or more nucleotides in both (a) and (b). In some instances, the genetic editing step (i) is mediated by a gene editing system. For example, the gene editing system comprises a Type V nuclease and a guide RNA (gRNA) targeting the STMN2 gene.

In some examples, the Type V nuclease is a Cas12i nuclease, which optionally comprises an amino acid sequence at least 90% identical to SEQ ID NO: 3. In specific examples, the Cas12i nuclease is a Cas12i2 nuclease comprising the amino acid sequence of SEQ ID NO: 3. In another example, the Cas12i nuclease is a Cas12i2 nuclease comprising the amino acid sequence of SEQ ID NO: 266.

In other examples, the Type V nuclease is a nuclease comprising the amino acid sequence of any one of SEQ ID NOs: 4-6, or a variant thereof, which may comprise an amino acid sequence at least 90% identical to SEQ ID NO: 4, 5, or 6. In one example, the Type V nuclease comprises the amino acid sequence of SEQ ID NO: 4. In another example, the Type V nuclease comprises the amino acid sequence of SEQ ID NO: 255. In another example, the Type V nuclease comprises the amino acid sequence of SEQ ID NO: 5. In yet another example, the Type V nuclease comprises the amino acid sequence of SEQ ID NO: 6.

Any of the gene editing systems provided herein may be used in the method for inhibiting aberrant splicing in a Stathmin-2 (STMN2) transcript as also disclosed herein.

In some embodiments, the gene editing system as disclosed herein may comprise two or more gRNAs targeting the STMN2 gene, for example, a first gRNA and a second gRNA. In some examples, the first gRNA induces a deletion of one or more nucleotides in the 3′ splice site and the second gRNA induces a deletion of one or more nucleotides in the downstream 10-nt window. In other examples, the first gRNA induces a deletion in both the 3′ splice site and the downstream 10-nt window and the second gRNA induces a deletion in the 3′ splice site. In yet other examples, the first gRNA induces a deletion in both the 3′ splice site and the downstream 10-nt window and the second gRNA induces a deletion in the downstream 10-nt window. In still other examples, each of the first gRNA and the second gRNA induces a deletion in both the 3′ splice site and the downstream 10-nt window.

In some embodiments, the cell for the gene editing disclosed herein is in cell culture. In some instances, the cell is from a human patient having amyotrophic lateral sclerosis (ALS) or frontotemporal dementia (FTD). In some examples, the cell is a brain cell, such as a neuron cell. In specific examples, the cell is a motor neuronal cell.

In other embodiments, the cell is a neuronal cell in a human patient having amyotrophic lateral sclerosis (ALS) or frontotemporal dementia (FTD).

In some embodiments, the method for inhibiting aberrant splicing in a STMN2 transcript disclosed herein comprises delivering the gene editing system to a subject in need thereof (e.g., a human patient having ALS or FTD). In some instances, the gene editing system is delivered to the subject by intracerebroventricular (ICV) injection. In some instances, the gene editing system is delivered to the subject by intrathecal injection.

In other aspects, the present disclosure features a genetically edited cell, comprising (a) a deletion of one or more nucleotides in a 3′ splice site of intron 1 of STMN2, wherein the 3′ splice site is adjacent to exon 2a; (b) a deletion of one or more nucleotides in a region of intron 1 that is adjacent to the 3′ splice site, or both (a) and (b). Such a genetically edited cell produces a reduced level of STMN2 transcripts including exon 2a and an increased level of functional STMN2 transcripts as compared with a non-edited counterpart. In some instances, the genetically edited cell is a human brain cell, such as a human neuron cell. In some examples, the human neuron cell is a motor neuronal cell. The genetically edited cell disclosed herein may be produced by any of the gene editing methods disclosed herein.

Further, the present disclosure provides a gene editing system, comprising: (a) a Type V CRISPR nuclease comprising an amino acid sequence at least 90% identical to SEQ ID NO: 4 or a nucleic acid encoding the Type V CRISPR nuclease; and (b) a guide RNA (gRNA) targeting a Stathmin-2 (STMN2) gene or a nucleic acid encoding the gRNA. Such gene editing system genetically modifies the STMN2 gene to inhibit production of STMN2 transcripts containing exon 2a. In some embodiments, the Type V CRISPR comprises the amino acid sequence of SEQ ID NO: 4. In some embodiments, the Type V CRISPR comprises the amino acid sequence of SEQ ID NO: 255.

In some embodiments, the gene editing system disclosed herein comprises a nucleic acid encoding the Type V CRISPR nuclease. In some examples, the nucleic acid is a vector, which comprises a nucleotide sequence encoding the Type V CRISPR nuclease, the nucleotide sequence being in operable linkage to a promoter. In some examples, the vector is an adeno-associated viral (AAV) vector (e.g., an AAVrh10 vector)). In some examples, the promoter is a synapsin 1 promoter. In some instances, the vector disclosed herein (e.g., an AAV vector such as an AAVrh10 vector) may further comprise a nucleotide sequence encoding the gRNA as disclosed herein.

Still further, the present disclosure provides a method for genetically editing a STMN2 gene, the method comprising contacting cells with any of the gene editing systems disclosed herein to allow for genetic editing of the STMN2 gene in the cells by the gene editing system.

In some embodiments, the method for inhibiting aberrant splicing in a STMN2 transcript disclosed herein comprises delivering the gene editing system to a subject in need thereof (e.g., a human patient). In some instances, the gene editing system is delivered to the subject by intracerebroventricular (ICV) injection. In some instances, the gene editing system is delivered to the subject by intrathecal injection.

Further, the present disclosure provides methods for treating a disease involving STMN2 aberrant splicing (e.g., ALS or FTD), comprising administering to a subject in need of the treatment any of the gene editing systems disclosed herein or modified cells produced by the gene editing system. Also provided herein are gene editing systems as disclosed herein for use in treating the target disease, as well as uses of the gene editing systems for manufacturing a medicament for use in treating the target disease.

Also provided herein are gene editing systems for use in genetically editing a STMN2 gene to inhibit production of exon 2a-containing splice variants and for use in treating a disease involving STMN2 aberrant splicing (e.g., ALS or FTD), as well as uses of such gene editing systems for manufacturing a medicament for use in the intended therapeutic applications.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1C include diagrams illustrating TDP-43 knockdown in SH-SY5Y cells. FIG. 1A shows a 1.4-fold TDP-43 knockdown in SH-SY5Y cells using siTDP-43 RNA. FIG. 1B shows that knockdown of TDP-43 in the presence of a control RNP complex (i.e., an RNP complex that does not target STMN2 exon 2a) resulted in a 60-fold increase in the STMN2 exon 2a splice variant. FIG. 1C shows knockdown of TDP-43 in the presence of a control RNP complex (i.e., an RNP complex that does not target STMN2 exon 2a) resulted in over a 90-fold decrease in the STMN2 full length isoform.

FIGS. 2A-2C include diagrams illustrating indel activity and splice motif disruption of the tested RNA guides in SH-SY5Y cells. FIG. 2A illustrates that RNA guides 55 and 56 showed the highest motif disruption among all tested guides. FIG. 2B shows a correlation between RNA guides that promoted STMN2 full length recovery and raw indels. FIG. 2C shows a correlation between RNA guides that promoted STMN2 full length recovery and motif disruption.

FIGS. 3A-3D include diagrams showing the impact on STMN2 transcript production by targeting the STMN2 exon 2a splicing site and TDP-43 binding site using Cas12i2. FIG. 3A shows 3′ splice site, 10-nt window, and the TBD-43 binding site in the corresponding region of the STMN2 gene. The nucleotide sequence presented in FIG. 3A is set forth as SEQ ID NO: 256. FIG. 3B shows that disruption of the TDP-43 binding site with RNA guide 12 recapitulated the effect of siRNA TDP-43 knockdown. In the absence of siRNA mediated TDP-43 knockdown and presence of RNA guide 12, splice site disruption with RNA guide 55 resulted in a decrease in the exon 2a splice variant (FIG. 3C) and an increase in full length STMN2 (FIG. 3D). NT: non-target guide. 12: Guide 12 (targeting the TDP-43 binding site). 55: Guide 55 (see Table 2).

FIGS. 4A-4B include diagrams illustrating representative plots for one dataset analyzed, showing the maximum correlation observed with a 10-nt window starting at amplicon position 106 (see SEQ ID NO:1). FIG. 4A shows correlation values between position indel rates and STMN2 recovery rates across guides (y-axis) for all amplicon positions (x-axis) are plotted with the position for maximum correlation (black dotted line). FIG. 4B shows scatter plot of guide position indels rates with a 10-nt window starting at position 106 (x-axis) and STMN2 recovery rates (y-axis).

FIGS. 5A-5G include diagrams showing the positions of indels induced by a particular guide within the STMN2 amplicon (x-axis). The number of NGS reads is on the y-axis. Minimal or no full-length STMN2 recovery was observed for guides that did not induce indels within either window. FIG. 5A: guides G1-G3. FIG. 5B: guides G4-G6. FIG. 5C: guides G7-G9. FIG. 5D: guides G10, G11, and G50. FIG. 5E: guides G51-G53. FIG. 5F: guides G54-G56. FIG. 5G: guides G57 and G58. The Indel Position base pair (bp) numbers at X-axis are relative to SEQ ID NO: 1. The 3′ splice site: positions 99-104 (region defined by the solid lines, having a sequence of TTGCAG); the identified optimal 10-nt window: 106-115 (region defined by the dash lines; having a sequence of ACTCGGCAGA (SEQ ID NO: 2)). See SEQ ID NO: 1 below.

FIG. 6 is a diagram showing RNA guides that produced >5% Motif Disruption (shown in bold in Table 6).

FIGS. 7A-7G include diagrams showing the positions of indels induced by a particular guide as indicated in association with Nuclease A within the STMN2 amplicon (x-axis). FIG. 7A: guide A_STMN2_Splice2a_1. FIG. 7B: guide A_STMN2_Splice2a_3. FIG. 7C: guide A_STMN2_Splice2a_2. FIG. 7D: guide A_STMN2_Splice2a_4. FIG. 7E: guide A_STMN2_Splice2a_5. FIG. 7F: guide A_STMN2_Splice2a_7. FIG. 7G: guide A_STMN2_Splice2a_6.

FIGS. 8A-8E include diagrams showing the positions of indels induced by a particular guide as indicated in association with Nuclease C within the STMN2 amplicon (x-axis). FIG. 8A: guide C_STMN2_Splice2a_36. FIG. 8B: guide C_STMN2_Splice2a_38. FIG. 8C: guide C_STMN2_Splice2a_37. FIG. 8D: guide C_STMN2_Splice2a_39. FIG. 8E: guide C_STMN2_Splice2a_43.

FIGS. 9A-9K include diagrams showing that editing the 3′ splicing site disclosed herein rescued increase in production of STMN2 exon 2A splice variant caused by knockdown of TDP-43 and positions of indels induced by a particular guide as indicated in association with Nuclease A in SH-SY5Y cells. FIG. 9A: is a diagram showing that a 3-fold TDP-43 knockdown was observed in SH-SY5Y cells using siTDP-43 RNA. FIG. 9B: a diagram showing that knockdown of TDP-43 in the presence of a non-targeting control RNP complex resulted in an 80-fold increase in the STMN2 exon 2A splice variant. FIG. 9C: a diagram showing that knockdown of TDP-43 in the presence of a non-targeting control RNP complex resulted in over a 7.5-fold decrease in the STMN2 full length transcript. FIG. 9D is a diagram showing the indel activity of Nuclease A in SH-SYSY cells. FIG. 9E: guide A_STMN2_Splice2a_1. FIG. 9F: guide A_STMN2_Splice2a_2. FIG. 9G: guide A_STMN2_Splice2a_3. FIG. 9H: guide A_STMN2_Splice2a_4. FIG. 9I: guide A_STMN2_Splice2a_5. FIG. 9J: guide A_STMN2_Splice2a_6. FIG. 9K: guide A_STMN2_Splice2a_7.

FIGS. 10A-10C include diagrams showing that the gene editing system comprising Nuclease A or Nuclease D and an exemplary gRNA targeting the 3′ splicing site successfully rescued increase in production of STMN2 exon 2A splice variant caused by knockdown of TDP-43. FIG. 10A: a diagram showing that a 3-fold and 5-fold TDP-43 knockdown was observed in SH-SY5Y cells using siTDP-43 RNA, when co-nucleofected with RNP comprising the Type V CRISPR nuclease variants as indicated. FIG. 10B: a diagram showing that knockdown of TDP-43 in the presence of a non-targeting control RNP complex resulted in a 120-fold and 130-fold increase in the STMN2 exon 2A splice variant when co-nucleofected with RNPs comprising the Type V CRISPR nuclease variants as indicated. FIG. 10C: a diagram showing that an 8-fold and 10-fold decrease in the STMN2 full length transcript was observed with siTDP-43 knockdown and RNPs comprising the Type V CRISPR nuclease variants as indicated.

FIGS. 11A-11P include diagrams illustrating splice motif disruption and the reversal of STMN2 mis-splicing in human motor neurons and positions of indels induced by a particular guide as indicated in association with Cas12i2 in motor neurons. FIGS. 11A-11B illustrate the inverse relationship between the decrease of the truncated exon2a STMN2 transcript and increase in the full length STMN2 transcript with motif disruption. FIG. 11C illustrates indel activity in motor neurons correlates with recovery of full length STMN2 transcript. gNT: not-targeting guide controls. FIG. 11D: guide STMN2_Splice2a_4 (G4). FIG. 11E: guide STMN2_Splice2a_8 (G8). FIG. 11F: guide STMN2_Splice2a_9 (G9). FIG. 11G: guide STMN2_Splice2a_10 (G10). FIG. 11H: guide STMN2_Splice2a_50 (G50). FIG. 11I: guide STMN2_Splice2a_51 (G51). FIG. 11J: guide STMN2_Splice2a_52 (G52). FIG. 11K: guide STMN2_Splice2a_53 (G53). FIG. 11L: guide STMN2_Splice2a_54 (G54). FIG. 11M: guide STMN2_Splice2a_55 (G55). FIG. 11N: guide STMN2_Splice2a_56 (G56). FIG. 11O: guide STMN2_Splice2a_57 (G57). FIG. 11P: guide STMN2_Splice2a_58 (G58).

FIGS. 12A-12L include diagrams illustrating splice motif disruption and the reversal of STMN2 mis-splicing by Nuclease A in human motor neurons and positions of indels included by a particular guide as indicated in association with Nuclease A in motor neurons. FIGS. 12A-12B illustrate the inverse relationship between the decrease of the truncated exon2a STMN2 transcript and increase in the full length STMN2 transcript with motif disruption. FIG. 12C illustrates indel activity in motor neurons correlates with recovery of full length STMN2 transcript. gNT: not-targeting guide controls. FIG. 12D illustrates STMN2 motif disruption in motor neurons analyzed by a digital droplet polymerase chain reaction (ddPCR) assay. FIG. 12E: guide A_STMN2_Splice2a_1. FIG. 12F: guide A_STMN2_Splice2a_2. FIG. 12G: guide A_STMN2_Splice2a_3. FIG. 12H: guide A_STMN2_Splice2a_4. FIG. 12I: guide A_STMN2_Splice2a_5. FIG. 12J: guide A_STMN2_Splice2a_6. FIG. 12K: guide A_STMN2_Splice2a_7. FIG. 12L: Control.

FIGS. 13A-13D include images and diagrams illustrating the phenotypic rescue in human motor neurons after the reversal of STMN2 mis-splicing by Cas12i2 and RNA guide G55. FIG. 13A shows representative images from the different conditions tested, in the presence or absence of TDP43 knockdown and a non-targeting control guide or guide 55. Cells stained with B3-tubulin and STMN2, nuclei stained with Hoechst. FIG. 13B and FIG. 13D illustrate the rescue in neurite length and the increase of STMN2-positive neurons observed after treatment with RNA guide G55, respectively. FIG. 13C illustrates that the RNA guide G55 treatment did not have an impact on the cell number. gNT: not-targeting guide controls.

FIGS. 14A-14G include diagrams showing genetic editing of STMN2 gene in mice by a gene editing system provided herein, which is delivered via an AAV vector. FIGS. 14A-14D are diagrams illustrating AAV vector genomes quantification, splice motif disruption and the reversal of STMN2 mis-splicing in vivo. FIG. 14A shows the number of VG per diploid genome observed in mice ICV-injected with four different vectors. FIGS. 14B-14D illustrate the motif disruption, decrease of the truncated exon2a STMN2 transcript, and increase in the full length STMN2 transcript in vivo after injection of the tested vectors, respectively. FIGS. 14E-14G are diagrams illustrating the correlation between editing percentage and STMN2 transcript levels in mice injected with Nuclease A+g4. FIG. 14E: correlation between editing percentage and exon2a STMN2 transcript. FIG. 14F: correlation between editing percentage and full length STMN2 transcript. FIG. 14G shows inverse relationship between exon 2a STMN2 transcript and full length STMN2 transcript. Ctrl: mice ICV-injected with vehicle.

### DETAILED DESCRIPTION OF THE INVENTION

Stathmin-2 (STMN2) (also known as SCG10) is a microtubule-associated protein enriched in brain. STMN2 plays import roles in promoting microtubule instability necessary for normal axonal outgrowth and regeneration. The expression of STMN2 is strongly regulated by nuclear transactive response DNA-binding protein 43 kDA (TDP-43). TDP-43 binds to a site within intron 1 of STMN pre-mRNA to regulate production of functional STMN2 transcript and thus functional STMN2 proteins. Certain diseases such as amyotrophic lateral sclerosis (ALS) and frontotemporal dementia (FTD) are associated with loss of nuclear TDP-43, which led to aberrant splicing of STMN2 to produce a non-functional transcript including a cryptic exon 2a. Melamed et al., Nat. Neurosci. 2019 22 (2): 180-190.

It is reported herein that disrupting a 3′ splice site in intron 1 (e.g., adjacent to the cryptic exon 2a) and/or a region downstream and adjacent to the 3′ splice site via gene editing significantly inhibited the aberrant splicing of STMN2 and improved production of functional STMN2 transcripts (encoding functional STMN2 proteins). Accordingly, provided herein are methods and gene editing systems (e.g., comprising Type V CRISPR nucleases) designed to inhibit or prevent aberrant splicing of STMN2. Such methods and gene editing systems could potentially alleviate motor neuron dysfunction in diseases involving the STMN2 aberrant splicing, such as ALS and FTD.

### I. Genetic Editing in STMN2 Gene for Inhibiting Aberrant Splicing

Human STMN2 gene is located at 8q21.13. Reference is made to Gene ID: 11075, which provides the sequence of the whole human STMN2 gene (ncbi.nlm.nih.gov/gene/11075), the relevant disclosures of which are incorporated by reference for the subject matter and purpose referenced herein.

Provided herein are methods for genetic editing at specific locations in the intron 1 of the STMN2 to inhibit or prevent occurrence of the aberrant splicing and to increase production of functional STMN2 transcripts and thus functional STMN2 proteins. As used herein, “aberrant splicing of STMN2” refers to the RNA splicing event that produces the non-functional transcript that includes the cryptic exon 2a. Due to the presence of a premature stop codon in exon 2a and premature poly(A) site (see below disclosures), the non-functional transcript would lead to production of a truncated, non-functional STMN2 protein.

### (a) Targeting Genetic Sites

In some embodiments, the location in intron 1 for genetic editing includes a 3′ splice site in the intron 1 of the STMN2 gene (e.g., the 3′ splicing site adjacent and upstream to the exon 2a sequence), a region in intron 1 that is adjacent and downstream to the 3′ splice site, or a combination thereof, the genetic editing leading to reduced or eliminated aberrant STMN2 splicing. The nucleotide sequence of a fragment of STMN2 intron 1 is provided below for reference purposes.

(SEQ ID NO: 1)
ACTTCCGAACTCATATACCTGGGGATTTTATTACTCTGGGAATTATG
TGTTCTGCCCCATCACTCTCTCTTAATTGGATTTTTAAAATTATATT
CATATTGCAGGACTCGGCAGAAGACCTTCGAGAGAAAGGTAGAAAAT
AAGAATTTGGCTCTCTGTGTGAGCATGTGTGCGTGTGTGCGAGAGAG
AGAGACAGACAGCCTGCCTAAGAAGAAATGAATGTGAATGCGGCTTG
TGGCACAGTTGACAAGGATGATAAATCAATAATGCAAGCTTACTATC
ATTTATGAATAGCAATACTGAAGAAATTAAAACAAAAGATTGCTGTC
TCAATATATCTTATATTTATTATTTACCAAATTATTCTAAGAGTATT
TCTTCCTGAATACCATGTGAGAAAATTCTTAAGAATTTATTGAGTAT
GACTGTATATTTGAAAAGAGTG

In the above sequence, the 3′ splice site TTGCAG is in boldface and underlined; the intron 1 region adjacent and downstream to the 3′ splice site, ACTCGGCAGA (SEQ ID NO: 2), also referred to as the optimal 10-nt window (a.k.a., optimal disruption window), is in boldface and italicized. The cryptic exon 2a starts from the “G” residue following the 3′ splice site. Further, the premature stop codon TAG (resulting in a truncated STMN2 protein when the aberrant splicing occurs) and a premature poly(A) site ATTAAA are underlined.

In some examples, the genetic editing method disclosed herein targets the 3′ splice site, e.g., leading to deletion of one or more nucleotides of the 3′ splice site, thereby disrupting its function as a 3′ splice site. In other examples, the genetic editing method disclosed herein targets the region adjacent to the 3′ splice site as disclosed herein, e.g., leading to deletion of one or more nucleotides within this region. In specific examples, the genetic editing method disclosed herein targets both the 3′ splice site and the downstream adjacent region, leading to deletions of one or more nucleotides in both regions.

Targeting the 3′ splice site, the optimal 10-nt window, or both as provided herein is expected to reduce the production of exon 2a-containing splice variants and increase the production of full-length STMN2 transcripts.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, or 10 nucleotides). In some embodiments, a first sequence is adjacent to a second sequence if the two sequences are separated by up to 2 nucleotides, up to 5 nucleotides, up to 8 nucleotides, or up to 10 nucleotides.

As used herein, the terms “upstream” and “downstream” refer to relative positions within a single nucleic acid (e.g., DNA) sequence. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence.

In other embodiments, the gene editing system provided herein (e.g., comprising a Type V nuclease having an amino acid sequence at least 90% identical to SEQ ID NO: 4) may target a genomic site within the STMN2 gene to reduce the production of exon 2a-containing splice variants. In some instances, such a gene editing system may target a genomic site within the STMN2 gene to disrupt expression of the STMN2 gene.

In other embodiments, the gene editing system provided herein (e.g., comprising a Type V nuclease having an amino acid sequence at least 90% identical to SEQ ID NO: 4) may target a genomic site within the STMN2 gene to reduce the production of exon 2a-containing splice variants. In some instances, such a gene editing system may target a genomic site within the STMN2 gene to disrupt expression of the STMN2 gene.

The genetic editing method may result in gene editing at the target location(s) as disclosed herein. As used herein, the term “edit” refers to one or more modifications introduced into one or more of the target locations in the STMN2 gene. The edit can be one or more substitutions, one or more insertions, one or more deletions, or a combination thereof. As used herein, the term “substitution” refers to a replacement of a nucleotide or nucleotides with a different nucleotide or nucleotides, relative to a reference sequence. As used herein, the term “insertion” refers to a gain of a nucleotide or nucleotides in a nucleic acid sequence, relative to a reference sequence. As used herein, the term “deletion” refers to a loss of a nucleotide or nucleotides in a nucleic acid sequence, relative to a reference sequence.

The genetic editing method as disclosed herein may be performed to a suitable cell (e.g., a cell that produces STMN2 and may produce or at risk for producing the non-functional transcript due to the aberrant splicing event) to edit the specific genetic locations in intron 1 of STMN2 as also disclosed herein. In some embodiments, the suitable cell can be a neutron cell, for example, obtained from a human patient having a disease involving STMN2 aberrant slicing such as ALS or FTD. In some instances, the genetic editing may be performed on cultured cells in vitro. Alternatively, the genetic editing may be performed in vivo to edit cells (e.g., neuron cells) in a subject in need thereof (e.g., a human patient having ALS or FTD).

### (ii) Genetic Editing Methods

The genetic editing of the STMN2 gene at the specific locations described herein may be achieved by a genetic editing system as known in the art, for example, a CRISPR/Cas-mediated gene editing system (e.g., involving a CRISPR nuclease such as a Type V nuclease), a zinc-finger nucleases (ZFN)-mediated gene editing system, or a transcription activator-like effector nucleases (TALEN)-mediated gene editing system. Additional examples include the DICE (dual integrase cassette exchange) system utilizing phiC31 and Bxb1 integrases.

In some embodiments, the genetic editing of the STMN2 gene as disclosed herein may be achieved using a gene editing system comprising a CRISPR nuclease and one or more guide RNAs targeting suitable genomic locations in the STMN2 gene to introduce the desired edits at the desired locations, including the 3′ splice site and/or the 10-nt downstream window as disclosed herein. “CRISPR nuclease” refers to an RNA-guided effector that is capable of binding a nucleic acid and introducing a single-stranded break or double-stranded break. In some embodiments, a CRISPR nuclease can be a Type II CRISPR nuclease, which refers to a nuclease comprising a RuvC domain and an HNH domain. The Type II nuclease can be a Type II-A nuclease, a Type II-B nuclease, or a Type II-C nuclease. In some embodiments, the Type II nuclease requires a tracrRNA. In some embodiments, the Type II nuclease is a Cas9 polypeptide. The Cas9 polypeptide can cleave a double-stranded DNA target or be a nickase. In other embodiments, a CRISPR nuclease can be a Type V CRISPR nuclease (see detailed disclosures below). In some embodiments, a CRISPR nuclease is an effector as described in Makarova et al. “Classification and Nomenclature of CRISPR-Cas Systems: Where from Here?” CRISPRJ. 1 (5): 325-36 (2018).

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Any of the nucleases disclosed herein may be delivered using a vector system, including, but not limited to, plasmid vectors, DNA minicircles, retroviral vectors, lentiviral vectors, adenovirus vectors, adeno-associated vectors, poxvirus vectors; herpesvirus vectors and adeno-associated virus vectors, and combinations thereof. Conventional viral and non-viral based gene transfer methods can be used to introduce nucleic acids encoding nucleases and gRNAs in cells (e.g., T cells). In some examples, the gene editing system or a component thereof (e.g., the nuclease therein) disclosed herein may be delivered via an AAV vector, which can be of a particular serotype capable of infecting neuron cells (e.g., AAVrh10). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell. Methods of non-viral delivery of nucleic acids include electroporation, lipofection, microinjection, biolistics, virosomes, liposomes, immunoliposomes, polycation or lipid: nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

Alternatively, the nucleases disclosed herein may be delivered by mRNAs, which may be associated with or encapsulated by lipid nanoparticles (LNPs).

To perform the gene editing method disclosed herein, a suitable gene editing system as disclosed herein can be delivered to or introduced into a population of cells (e.g., neuron cells). In some instances, cells comprising the desired genetic editing may be collected and optionally cultured and expanded in vitro.

### II. Type V Nuclease-Containing Gene Editing Systems

In some aspects, the present disclosure provides a CRISPR Type V-nuclease-mediated gene editing system for introducing the edits (e.g., deletions) at the desired genetic locations in the STMN2 gene as disclosed herein to introduce gene editing into the desired locations in the STMN2 gene, including the 3′ splice site adjacent (upstream) to the cryptic exon 2a and/or the downstream optimal 10-nt window in intron 1, thereby reducing or preventing the inclusion of the cryptic exon 2a, as well as increasing production of functional STMN2 transcripts. In some embodiments, the CRISPR Type V-nuclease-mediated gene editing system comprises a Type V nuclease or a nucleic acid encoding the nuclease and one or more RNA guides targeting suitable genomic sites within the STMN2 gene (e.g., in intron 1) or one or more nucleic acids encoding the one or more gRNAs as also disclosed herein.

### (i) Type V Nucleases

As used herein, the terms “Type V” and “Type V nuclease” refer to an RNA-guided CRISPR nuclease with a RuvC domain. In some embodiments, a Type V nuclease does not require a tracrRNA. In some embodiments, a Type V nuclease requires a tracrRNA. In some embodiments, the Type V nuclease is a Cas12 polypeptide, such as a Cas12a (Cpf1), Cas12b (C2c1), Cas12c, Cas12d, Cas12e, Cas12f, Cas12h, Cas12i, or Cas12j (CasPhi) polypeptide.

### (a) Cas12i Nucleases

“Cas12i nucleases” (also referred to herein as Cas12i) refers to a polypeptide that binds to a target sequence on a target nucleic acid specified by an RNA guide, wherein the polypeptide has at least some amino acid sequence homology to a wild-type Cas12i nuclease. Such Cas12i polypeptides are known in the art or disclosed herein. See, e.g., WO/2021/202800 and WO2022256440, the relevant disclosures of each of which are incorporated by reference for the subject matter and purpose disclosed herein.

In some embodiments, the Cas12i nuclease is a Cas12i2 polypeptide. In one example, the Cas12i2 polypeptide for use in the gene editing system and method disclosed herein can comprise one of the following amino acid sequences:

(SEQ ID NO: 3)
MSSAIKSYKS VLRPNERKNQ LLKSTIQCLE DGSAFFFKML
QGLFGGITPE IVRFSTEQEK QQQDIALWCA VNWFRPVSQD
SLTHTIASDN LVEKFEEYYG GTASDAIKQY FSASIGESYY
WNDCRQQYYD LCRELGVEVS DLTHDLEILC REKCLAVATE
SNQNNSIISV LFGTGEKEDR SVKLRITKKI LEAISNLKEI
PKNVAPIQEI ILNVAKATKE TFRQVYAGNL GAPSTLEKFI
AKDGQKEFDL KKLQTDLKKV IRGKSKERDW CCQEELRSYV
EQNTIQYDLW AWGEMFNKAH TALKIKSTRN YNFAKQRLEQ
FKEIQSLNNL LVVKKLNDFF DSEFFSGEET YTICVHHLGG
KDLSKLYKAW EDDPADPENA IVVLCDDLKN NFKKEPIRNI
LRYIFTIRQE CSAQDILAAA KYNQQLDRYK SQKANPSVLG
NQGFTWTNAV ILPEKAQRND RPNSLDLRIW LYLKLRHPDG
RWKKHHIPFY DTRFFQEIYA AGNSPVDTCQ FRTPRFGYHL
PKLTDQTAIR VNKKHVKAAK TEARIRLAIQ QGTLPVSNLK
ITEISATINS KGQVRIPVKF RVGRQKGTLQ IGDRFCGYDQ
NQTASHAYSL WEVVKEGQYH KELGCFVRFI SSGDIVSITE
NRGNQFDQLS YEGLAYPQYA DWRKKASKFV SLWQITKKNK
KKEIVTVEAK EKFDAICKYQ PRLYKFNKEY AYLLRDIVRG
KSLVELQQIR QEIFRFIEQD CGVTRLGSLS LSTLETVKAV
KGIIYSYFST ALNASKNNPI SDEQRKEFDP ELFALLEKLE
LIRTRKKKQK VERIANSLIQ TCLENNIKFI RGEGDLSTTN
NATKKKANSR SMDWLARGVF NKIRQLAPMH NITLFGCGSL
YTSHQDPLVH RNPDKAMKCR WAAIPVKDIG DWVLRKLSQN
LRAKNRGTGE YYHQGVKEFL SHYELQDLEE ELLKWRSDRK
SNIPCWVLQN RLAEKLGNKE AVVYIPVRGG RIYFATHKVA
TGAVSIVFDQ KQVWVCNADH VAAANIALTG KGIGEQSSDE
ENPDGSRIKL QLTS
(SEQ ID NO: 266)
MSSAIKSYKSVLRPNERKNOLLKSTIQCLEDGSAFFFKMLQGLFGGI
TPEIVRFSTEQEKQQQDIALWCAVNWFRPVSQDSLTHTIASDNLVEK
FEEYYGGTASDAIKQYFSASIGESYYWNDCRQQYYDLCRELGVEVSD
LTHDLEILCREKCLAVATESNQNNSIISVLFGTGEKEDRSVKLRITK
KILEAISNLKEIPKNVAPIQEIILNVAKATKETFRQVYAGNLGAPST
LEKFIAKDGQKEFDLKKLQTDLKKVIRGKSKERDWCCQEELRSYVEQ
NTIQYDLWAWGEMFNKAHTALKIKSTRNYNFAKQRLEQFKEIQSLNN
LLVVKKLNDFFDSEFFSGEETYTICVHHLGGKDLSKLYKAWEDDPAD
PENAIVVLCDDLKNNFKKEPIRNILRYIFTIRQECSAQDILAAAKYN
QQLDRYKSQKANPSVLGNQGFTWTNAVILPEKAQRNDRPNSLDLRIW
LYLKLRHPDGRWKKHHIPFYDTRFFQEIYAAGNSPVDTCQFRTPRFG
YHLPKLTDQTAIRVNKKHVKAAKTEARIRLAIQQGTLPVSNLKITEI
SATINSKGQVRIPVKFRVGRQKGTLQIGDRFCGYDQNQTASHAYSLW
EVVKEGQYHKELRCRVRFISSGDIVSITENRGNQFDQLSYEGLAYPQ
YADWRKKASKFVSLWQITKKNKKKEIVTVEAKEKFDAICKYQPRLYK
FNKEYAYLLRDIVRGKSLVELQQIRQEIFRFIEQDCGVTRLGSLSLS
TLETVKAVKGIIYSYFSTALNASKNNPISDEQRKEFDPELFALLEKL
ELIRTRKKKQKVERIANSLIQTCLENNIKFIRGEGDLSTTNNATKKK
ANSRSMDWLARGVFNKIRQLATMHNITLFGCGSLYTSHQDPLVHRNP
DKAMKCRWAAIPVKDIGDWVLRKLSQNLRAKNRGTGEYYHQGVKEFL
SHYELQDLEEELLKWRSDRKSNIPCWVLQNRLAEKLGNKEAVVYIPV
RGGRIYFATHKVATGAVSIVFDQKQVWVCNADHVAAANIALTGKGIG
RQSSDEENPDGGRIKLQLTS

In some instances, the Cas12i2 polypeptide may comprise an amino acid sequence that is at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, or at least 99% identical to SEQ ID NO: 3 or SEQ ID NO: 266. In one example, the Cas12i2 polypeptide comprises the amino acid sequence of SEQ ID NO: 3. In another example, the Cas12i2 polypeptide comprises the amino acid sequence of SEQ ID NO: 266.

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, word length=3 to obtain amino acid sequences homologous to the protein molecules of the invention. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

In some instances, the Cas12i2 polypeptide may comprise one or more conservative amino acid substitutions as compared with SEQ ID NO: 3. In some instances, the Cas12i2 polypeptide may comprise one or more conservative amino acid substitutions as compared with SEQ ID NO: 266. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

### (b) Other Type V Nucleases

Other Type V nucleases can also be used in the gene editing systems and methods disclosed herein. Exemplary suitable Type V nucleases can be found in WO2019178427, WO2021202800, WO2021050534, WO2022192391, WO2024020567, WO2022192381, WO2024020557, WO2020018142, and WO2023039472, the relevant disclosures of each of which are incorporated by reference for the subject matter and purpose referenced herein. Specific examples of Type V nucleases are provided in Table 1 below, all of which are within the scope of the present disclosure.

In some instances, the Type V nuclease may comprise an amino acid sequence that is at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, or at least 99% identical to SEQ ID NO: 4. Alternatively or in addition, the Type V nuclease may comprise one or more conservative amino acid substitutions relative to SEQ ID NO: 4. In specific examples, the Type V nuclease comprises the amino acid sequence of SEQ ID NO: 4 (Nuclease A listed in Table 1 below). In specific examples, the Type V nuclease comprises the amino acid sequence of SEQ ID NO: 255 (Nuclease D listed in Table 1 below).

In some instances, the Type V nuclease may comprise an amino acid sequence that is at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, or at least 99% identical to SEQ ID NO: 5. Alternatively or in addition, the Type V nuclease may comprise one or more conservative amino acid substitutions relative to SEQ ID NO: 5. In specific examples, the Type V nuclease comprises the amino acid sequence of SEQ ID NO: 5 (Nuclease B listed in Table 1 below).

In some instances, the Type V nuclease may comprise an amino acid sequence that is at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, or at least 99% identical to SEQ ID NO: 6. Alternatively or in addition, the Type V nuclease may comprise one or more conservative amino acid substitutions relative to SEQ ID NO: 6 (Nuclease C listed in Table 1 below).

TABLE 1
Amino Acid Sequences of Exemplary Type V Nuclease

| | | SEQ |
|---|---|---|
| Nuclease | Amino Acid Sequence | ID NO |
| Nuclease A | MIKSIQLKVKGECRITKDVINEYKEYYNNCSRWIKNNLTSITIGEMAKFLQSL | 4 |
| | SDKEVAYRSMGLSDEWKDKPLYHLFTKKYHTKNADNLLYYYIKEKNLDGYKGN | |
| | TLNISNTSFRQFGYFKLVVSNYRTKIRTLNCKIKRKKIDADSTSEDIEMQVMY | |
| | EIIKYSLNKKSDWDNFISYIENVENPNIDNINRYKLLRECFCENENMIKNKLE | |
| | LLSVEQLKKFGGCIMKPHINSMTINIQDFKIEEKENSLGFILHLPLNKKQYQI | |
| | ELLGNRQIKKGTKEIHETLVDITNTHGENIVFTIKNDNLYIVFSYRSEFEKEE | |
| | VNFAKTVGLDVNFKHAFFVGSEKDNCHLDGYINLYKYLLEHDEFTNLLTEDER | |
| | KDYEELSKVVTFCPFENQLLFARYNKMSKFCKKEQVLSKLLYALQKKLKDENR | |
| | TKEYIYVSCVNKLRAKYVSYFILKEKYYEKQKEYDIEMGFVDDSTESKESMDK | |
| | RRTEYPFRNTPVANELLSKLNNVQQDINGCLKNIINYIYKIFEQNGYKVVALE | |
| | NLENSNFEKKQVLPTIKSLLKYHKLENQNVNDIKASDKVKEYIENGYYELMTN | |
| | ENNEIVDAKYTEKGAMKVKNANFFNLMMKSLHFASVKDEFVLLSNNGKTQIAL | |
| | VPSEFTSQMDSTDHCLYMKKNDKGKLVKADKKEVRTKQERHINGLNADFNAAN | |
| | NIKYIVENEVWRGIFCTRPKKTEYNVPSLDTTKKGPSAILNMLKKIGAIKVLE | |
| | TEK | |
| Nuclease B | MTTKQVKSIVLKVKNTNECPITKDVINEYKKYYNICSEWIKDNLTSITIGDIA | 5 |
| | SFLKEARNKDTIPTYINMGLSEEWKYKPIYHLFTDRYHEKSANNLLYAYFKEK | |
| | NLDCYNGNILNLSETYYRRNGYFKSVVGNYRTKIRTLNYKIKRKNVDENSTNE | |
| | DIELQVMYEIAKRKLNIKKDWENYISYIENVENINIKNIDRYNLLYKHFCENE | |
| | STINCKMELLSVEQLKEFGGCVMKQHINSMTINIQDFKIENKENSLGFILNLP | |
| | LNKKKYQIELWGNRQIKKGNKDNYKTLVDFINTYGQNIIFTIKNNKIYVVFSY | |
| | ECELKEKEINFDKIVGIDVNFKHALFVASERDKNPLQGNNQLKGYINLYGYLL | |
| | EHNEFTSLLTKEELDIYKEIAKGVTFCPLEYNLLFTRIENKGGKSNDKEQVLS | |
| | KLLYSLQIKLKNENKIQEYIYVSCVNKLRAKYVSYFILKEKYYEKQKEYDIEM | |
| | GFTDDSTESKESMDKRRLEFPFRNTQIANGFLEKLSNVQQDINGCLKNIINYA | |
| | YKVFEQNGFGVIALENLENSNFEKTQVLPTIKSLLRYHKLRNQNINNINASDK | |
| | VKEYIEKEYYELTTNENNEIVDAKYTKKGIIKVKKANFFNLMMKSLHFASNKD | |
| | EFILLSNNGKTQIALVPSEYTSQMDSIEHCLYVDKNGKKVDKKKVRQKQETHI | |
| | NGLNADFNAANNIKYIIENENLRKLFCGKLKVSGYNTPILRATKKGQFNILAE | |
| | LKKQNKIKIFEIEK | |
| Nuclease C | MGAARRRNPKVAAARKGKPPPKATGNCRNYRYGAHEPIANLDKVLDEMRGAHD | 6 |
| | LRNVLTCINRARSEMITAALGEHQSYKKATADLAALHQRRDKLEAQIRQQNSA | |
| | SRKRLGRHSPLSSELDTVRKRIDEGRTALKKLRRKLLKKDPALKAVVEAADDM | |
| | AKRETTRAEDACGLYWCTRNEQTGKRAKLRRFKKWRDSEATISVQIPGGLTVE | |
| | QLLGGENNQARLELRPEGVWVQGARKRKVEPAEAARNKLRLDEDGYPMRKLGT | |
| | AILHLRCMSDEDGKPIWAEVPITYHREIPADAKIKRCYLHRFRVGNRYHWSVR | |
| | FSLERGKKGDDSWLHPRVATTGTAAIDIGWRWFPDRLRVAVWAGSDGAEGELC | |
| | LPKWWLDEMYSVRLDQRERDVLFNEIVSLVLPWFRSRRGELSDYVVQAIKTMH | |
| | SWRDKGRLAALSMRWRDDLAADPGANPAHVAMSIRLEEWRKRDKHIWCEEVNL | |
| | RSQLQGSRKDLYRRFAAMLTSRYGRIVVEEFRLSAVQKLPPASIDDGTYSRVK | |
| | RHKGDAACSHLVGALKDAARQLDKKNPKWTTKRCHVCGKTERKWENPGELEHT | |
| | CKHCGVLWDRDVNAARNILAASGVAVDWTRPPLAPAARMTYPQVENREMRRSR | |
| | RRKEALETTRASGDRQTA | |
| Nuclease D | MIKSIQLKVKGECRITKDVINEYKEYYNNCSRWIKNNLTSITIGEMAKFLQSL | 255 |
| | SDKEVAYRSMGLSDEWKDKPLYHLFTKKYHTKNADNLLYYYIKEKNLDGYKGN | |
| | TLNISNTSFRQFGYFKLVVSNYRTKIRTLNCKIKRKKIDADSTSEDIEMQVMY | |
| | EIIKYSLNKKSDWDNFISYIENVENPNIDNINRYKLLRECFCENENMIKNKLE | |
| | LLSVEQLKKFGGCIMKPHINSMTINIQDFKIEEKENSLGFILHLPLNKKQYQI | |
| | ELLGNRQIKKGTKEIHETLVDITNTHGENIVFTIKNDNLYIVFSYRSEFEKEE | |
| | VNFAKTVGLDVNFKHAFFVTSEKDNCHLDGYINLYKYLLEHDEFTNLLTEDER | |
| | KDYEELSKVVTFCPFENQLLFARYNKMSKFCKKEQVLSKLLYALQKKLKDENR | |
| | TKEYIYVSCVNKLRAKYVSYFILKEKYYEKQKEYDIEMGFVDDSTESKESMDK | |
| | RRTEYPFRNTPVANELLSKLNNVQQDINGCLKNIINYIYKIFEQNGYKVVALE | |
| | NLENSNFEKKQVLPTIKSLLKYHKLENQNVNDIKASDKVKEYIENGYYELMTN | |
| | ENNEIVDAKYTEKGAMKVKNANFFNLMMKSLHFASVKDEFVLLSNNGKTQIAL | |
| | VPSEFTSQMDSTDHCLYMKKNDKGKLVKADKKEVRTKQERHINGLNADFNAAN | |
| | NIKYIVENEVWRGIFCTRPKKTEYNVPSLDTTKKGPSAILNMLKKIEAIKVLE | |
| | TEK | |

Any of the Type V nuclease polypeptides provided herein, e.g., the Cas12i2 nuclease or the other Type V nucleases provided herein, may comprise one or more nuclear localization signals (NLS), for example, at the N-terminus, at the C-terminus, or both.

### (c) Preparation of Type V Nucleases

In some embodiments, a Type V nuclease such as a Cas12i2 polypeptide or other Type V nucleases as disclosed herein (as well as any suitable nucleases for use in gene editing as known in the art or disclosed herein) can be prepared by (a) culturing host cells such as bacteria cells or mammalian cells, capable of producing the proteins, isolating the proteins thus produced, and optionally, purifying the proteins. The nucleases can be also prepared by (b) a known genetic engineering technique, specifically, by isolating a gene encoding the nuclease from bacteria, constructing a recombinant expression vector, and then transferring the vector into an appropriate host cell that expresses guide RNAs that complexes with the nucleases in the host cell. Alternatively, the nuclease can be prepared by (c) an in vitro coupled transcription-translation system and then complexes with guide RNAs.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

### Vectors

The gene editing system disclosed herein may comprise a nucleic acid encoding the Type V CRISPR nuclease. In some examples, the nucleic acid is a vector, which comprises a nucleotide sequence encoding the Type V CRISPR nuclease. In some instances, the nucleotide sequence encoding the Type V CRISPR nuclease may be in operable linkage to a promoter (e.g., a synapsin 1 promoter). In some examples, the vector is an adeno-associated viral (AAV) vector, for example, an AAV vector of a suitable serotype that can infect neuron cells. In one example, the AAV vector may be an AAVrh10 vector.

The present disclosure provides one or more vectors for expressing the Type V nucleases disclosed herein (as well as other suitable nucleases as also disclosed herein). In some embodiments, a vector disclosed herein includes a nucleotide sequence encoding the nuclease. The present disclosure also provides one or more vectors encoding the guide RNA. In some embodiments, the vector comprises a U6 promoter, synapsin promoter, a Pol II promoter, and/or a Pol III promoter.

Expression of natural or synthetic polynucleotides is typically achieved by operably linking a polynucleotide encoding the gene of interest, e.g., nucleotide sequence encoding the Type V nucleases to a promoter and incorporating the construct into an expression vector. The expression vector is not particularly limited as long as it includes a polynucleotide encoding the nuclease and/or the guide RNA and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses useful as vectors include, but are not limited to, phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the polypeptide(s) from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In some embodiments, the expression vectors provided herein may comprise a nucleotide sequence encoding any of the Type V nucleases disclosed herein (e.g., SEQ ID NO: 3, SEQ ID NO: 266, or SEQ ID NO: 4) and a nucleotide sequence encoding a guide RNA as also disclosed herein. For example, the expression vector may comprise a nucleotide sequence encoding the Type V nuclease of SEQ ID NO: 3 or SEQ ID NO: 266 and a nucleotide sequence encoding the guide of G53, G55, or G56. In other examples, the expression vector may comprise a nucleotide sequence encoding the Type V nuclease of SEQ ID NO: 4 or SEQ ID NO: 255 and a nucleotide sequence encoding the guide of A_STMN2_Splice2a_4 or A_STMN2_Splice2a_3. In some instances, the nuclease-encoding sequence and the gRNA-encoding sequence may each be in operable linkage to a suitable promoter. For example, the nuclease-encoding sequence may be in operable linkage to a synapsin 1 promoter and the gRNA-encoding sequence may be in operable linkage to a U6 promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the polypeptide(s) of the present invention has been transferred into the host cells and then expressed without fail.

The preparation method for recombinant expression vectors is not particularly limited, and examples thereof include methods using a plasmid, a phage or a cosmid.

### Methods of Expression

The present disclosure includes a method for expressing the Type V nuclease (and other suitable nucleases) in host cells of interest.

In some embodiments, a host cell described herein is used to express the Type V nuclease (and other suitable nucleases) and/or the guide RNA. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Type V nuclease, and/or the guide RNA. After expression of the nuclease and/or the guide RNA, the host cells can be collected and the nuclease and/or the guide RNA purified from the cultures etc. according to conventional methods (for example, filtration, centrifugation, cell disruption, gel filtration chromatography, ion exchange chromatography, etc.).

A variety of methods can be used to determine the level of production of a mature Type V nuclease (or other suitable nucleases) and/or the guide RNA in a host cell. Such methods include, but are not limited to, for example, methods that utilize either polyclonal or monoclonal antibodies specific for the proteins or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the nuclease and/or the guide RNAs in a cell (e.g., in a neuron cell). Such a method may comprise providing a polyribonucleotide encoding the nuclease and/or the guide RNAs to a suitable cell (e.g., a neuron cell). Expression of the nuclease and the guide RNAs in the cell could lead to the desired gene editing in the cell. In some examples, the Type V nuclease and the gRNA as disclosed herein may be delivered to the host cell (e.g., a neuron cell such as a motor neuron cell) via one or more viral vector(s), for example, an AAV vector (e.g., AAVrh10). In some examples, the expression of the Type V nuclease in neuron cells may be under the control of a suitable promoter, such as a synapsin 1 promoter. In some examples, the expression of the gRNA nuclease in neuron cells may be under the control of a suitable promoter, such as a synapsin 1 promoter or a U6 promoter.

(ii) Guide RNAs (gRNAs)

Any of the gene editing systems disclosed herein also comprises one or more guide RNAs (gRNAs) targeting suitable genomic sites in the STMN2 gene (e.g., in intron 1 of the STMN2 gene), leading to gene editing at the desired STMN2 locations, including the 3′ splice site in intron 1 (for inclusion of the cryptic exon 2a) and the 10-nt downstream window as disclosed herein. See SEQ ID NO: 1 provided above. The gRNAs mediate cleavage of a target nucleic acid via the CRISPR nuclease also contained in the gene editing system. The RNA guide (or a gRNA) comprises a nuclease binding sequence and a DNA-binding sequence (a spacer). The nuclease binding sequence may comprise one or more binding sites that can be recognized by the CRISPR nuclease for binding. In some instances, the gRNA is a single RNA molecule comprising both the nuclease binding sequence and a spacer. Alternatively, the gRNA may comprise the nuclease binding sequence and the spacer as two separate RNA molecules.

As used herein, the terms “RNA guide” or “RNA guide sequence” refer to an RNA molecule that facilitates the targeting of a CRISPR nuclease described herein to a genomic site of interest. For example, an RNA guide can be a molecule that recognizes (e.g., binds to) a site in a non-PAM strand that is complementary to a target sequence in the PAM strand, e.g., designed to be complementary to a specific nucleic acid sequence. An RNA guide comprises a spacer and a nuclease binding sequence (e.g., a direct repeat (DR) sequence). The terms CRISPR RNA (crRNA), pre-crRNA and mature crRNA are also used herein to refer to an RNA guide. The 5′ end or 3′ end of an RNA guide may be fused to an RT donor RNA as disclosed herein.

As used herein, the term “protospacer adjacent motif” or “PAM sequence” refers to a DNA sequence adjacent to a target sequence. In some embodiments, a PAM sequence is required for enzyme activity. In a double-stranded DNA molecule, the strand containing the PAM motif is called the “PAM-strand” and the complementary strand is called the “non-PAM strand.” The RNA guide binds to a site in the non-PAM strand that is complementary to a target sequence disclosed herein, and the PAM sequence as described herein is present in the PAM-strand.

As used herein, the term “PAM strand” refers to the strand of a target nucleic acid (double-stranded) that comprises a PAM motif. In some embodiments, the PAM strand is a coding (e.g., sense) strand. In other embodiments, the PAM strand is a non-coding (e.g., antisense strand). The term “non-PAM strand” refers to the complementary strand of the PAM strand.

A guide RNA typically comprises a spacer sequence and a scaffold sequence. The spacer sequence (a.k.a., a DNA-binding sequence) is the RNA equivalent of the target sequence (a DNA sequence). The spacer contains a sequence capable of binding to the non-PAM strand via base-pairing at the site complementary to the target sequence (in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference. The scaffold sequence comprises a motif recognizable by a nuclease (e.g., a Type V nuclease as disclosed herein)

As used herein, the term “target sequence” refers to a DNA fragment adjacent to a PAM motif (on the PAM strand). The complementary region of the target sequence is on the non-PAM strand. A target sequence may be immediately adjacent to the PAM motif. Alternatively, the target sequence and the PAM may be separated by a small sequence segment (e.g., up to 5 nucleotides, for example, up to 4, 3, 2, or 1 nucleotide). A target sequence may be located at the 3′ end of the PAM motif or at the 5′ end of the PAM motif, depending upon the CRISPR nuclease that recognizes the PAM motif, which is known in the art. For example, a target sequence is located at the 3′ end of a PAM motif for a Cas12i polypeptide (e.g., a Cas12i2 polypeptide such as those disclosed herein) or the other Type V nucleases such as those disclosed herein (see Table 1 above).

As used herein, the term “complementary” refers to a first polynucleotide (e.g., a spacer sequence of an RNA guide) that has a certain level of complementarity to a second polynucleotide (e.g., the complementary sequence of a target sequence) such that the first and second polynucleotides can form a double-stranded complex via base-pairing to permit an effector polypeptide (e.g., a Type V nuclease or a variant thereof) that is complexed with the first polynucleotide to act on (e.g., cleave) the second polynucleotide. In some embodiments, the first polynucleotide may be substantially complementary to the second polynucleotide, i.e., having at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second polynucleotide. In some embodiments, the first polynucleotide is completely complementary to the second polynucleotide, i.e., having 100% complementarity to the second polynucleotide.

### Nuclease Binding Sequence (Direct Repeat)

In some embodiments, the nuclease binding sequence in a gRNA disclosed herein is a CRISPR nuclease binding sequence (e.g., the nuclease binding sequence is capable of binding to a Type V nuclease or a Type II nuclease). In some embodiments, the nuclease binding sequence comprises a direct repeat sequence. In certain embodiments, the nuclease binding sequence includes a direct repeat sequence linked to a DNA-binding sequence (e.g., a DNA-targeting sequence or spacer). In some embodiments, the nuclease binding sequence includes a direct repeat sequence and a DNA-binding sequence or a direct repeat-DNA-binding sequence-direct repeat sequence. In some embodiments, the nuclease binding sequence includes a truncated direct repeat sequence and a DNA-binding sequence, which is typical of processed or mature crRNA.

In the embodiments where the nuclease binding sequence is a direct repeat for a publicly available CRISPR nuclease, those direct repeat sequences are known in the art. In some embodiments, direct repeat sequences capable of binding a CRISPR nuclease are any of those disclosed in WO2021055874, WO2020206036, WO2020191102, WO2020186213, WO2020028555, WO2020033601, WO2019126762, WO2019126774, WO2019071048, WO2019018423, WO2019005866, WO2018191388, WO2018170333, WO2018035388, WO2018035387, WO2017219027, WO2017189308, WO2017184768, WO2017106657, WO2016205749, WO2017070605, WO2016205764, WO2016205711, WO2016028682, WO2015089473, WO2014093595, WO2015089427, WO2014204725, WO2015070083, WO2014093655, WO2014093694, WO2014093712, WO2014093635, WO2021133829, WO2021007177, WO2020197934, WO2020181102, WO2020181101, WO2020041456, WO2020023529, WO2020005980, WO2019104058, WO2019089820, WO2019089808, WO2019089804, WO2019089796, WO2019036185, WO2018226855, WO2018213351, WO2018089664, WO2018064371, WO2018064352, WO2017106569, WO2017048969, WO2016196655, WO2016106239, WO2016036754, WO2015103153, WO2015089277, WO2014150624, WO2013176772, WO2021119563, WO2021118626, WO2020247883, WO2020247882, WO2020223634, WO2020142754, WO2020086475, WO2020028729, WO2019241452, WO2019173248, WO2018236548, WO2018183403, WO2017027423, WO2018106727, WO2018071672, WO2017096328, WO2017070598, WO2016201155, WO2014150624, WO2013098244, WO2021113522, WO2021050534, WO2021046442, WO2021041569, WO2021007563, WO2020252378, WO2020180699, WO2020018142, WO2019222555, WO2019178428, WO2019178427, or WO2019006471, which are incorporated by reference for the subject matter and purpose referenced herein.

In some embodiments, the direct repeat sequence of the RNA guide has a length of between 12-100, 13-75, 14-50, or 15-40 nucleotides (e.g., 15, 16, 17, 18, 19, 20, 21, 22, 23, 24, 25, 26, 27, 28, 29, 30, 31, 32, 33, 34, 35, 36, 37, 38, 39, or 40 nucleotides).

In some examples, the nuclease binding sequence is capable of binding to a Cas12i polypeptide (e.g., SEQ ID NO: 3 or SEQ ID NO: 266 disclosed herein or a variant thereof). See Table 2 below. For example, a direct repeat sequence recognizable by Cas12i2 nucleases (e.g., SEQ ID NO: 3 or SEQ ID NO: 266) may comprise (e.g., consisting of) the nucleotide sequence of 5′-AGAAAUCCGUCUUUCAUUGACGG-3′ (SEQ ID NO: 36).

In other examples, the nuclease binding site is capable of binding to the other Type V nucleases disclosed herein, e.g., capable of binding to SEQ ID NO: 4, 5, or 6, or a variant thereof (e.g., SEQ ID NO: 255). See Tables 3-5 below. For example, a direct repeat sequence recognizable by Type V nuclease A and its variants (e.g., SEQ ID NO: 4 or SEQ ID NO: 255) may comprise (e.g., consisting of) the nucleotide sequence of 5′-CUUGUUGUAUAUGUCCUUUUAUAGGUAUUAAACAAC-3′ (SEQ ID NO: 56).

### DNA-Binding Sequence (Spacer)

The RNA guide also comprises a DNA-binding sequence (also known as a spacer). A spacer may have a length of from about 7 nucleotides to about 100 nucleotides. For example, the spacer can have a length of from about 7 nucleotides to about 80 nucleotides, from about 7 nucleotides to about 50 nucleotides, from about 7 nucleotides to about 40 nucleotides, from about 7 nucleotides to about 30 nucleotides, from about 7 nucleotides to about 25 nucleotides, from about 7 nucleotides to about 20 nucleotides, or from about 7 nucleotides to about 19 nucleotides. For example, the spacer can have a length of from about 7 nucleotides to about 20 nucleotides, from about 7 nucleotides to about 25 nucleotides, from about 7 nucleotides to about 30 nucleotides, from about 7 nucleotides to about 35 nucleotides, from about 7 nucleotides to about 40 nucleotides, from about 7 nucleotides to about 45 nucleotides, from about 7 nucleotides to about 50 nucleotides, from about 7 nucleotides to about 60 nucleotides, from about 7 nucleotides to about 70 nucleotides, from about 7 nucleotides to about 80 nucleotides, from about 7 nucleotides to about 90 nucleotides, from about 7 nucleotides to about 100 nucleotides, from about 10 nucleotides to about 25 nucleotides, from about 10 nucleotides to about 30 nucleotides, from about 10 nucleotides to about 35 nucleotides, from about 10 nucleotides to about 40 nucleotides, from about 10 nucleotides to about 45 nucleotides, from about 10 nucleotides to about 50 nucleotides, from about 10 nucleotides to about 60 nucleotides, from about 10 nucleotides to about 70 nucleotides, from about 10 nucleotides to about 80 nucleotides, from about 10 nucleotides to about 90 nucleotides, or from about 10 nucleotides to about 100 nucleotides.

In some embodiments, the spacer in the RNA guide may be generally designed to have a length of between 7 and 50 nucleotides or between 15 and 35 nucleotides (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides) and be complementary to a specific target sequence.

In some embodiments, the DNA-binding sequence has at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a target sequence as described herein and is capable of binding to the complementary region of the target sequence via base-pairing.

In some embodiments, the DNA-binding sequence comprises only RNA bases. In some embodiments, the DNA-binding sequence comprises a DNA base (e.g., the spacer comprises at least one thymine). In some embodiments, the DNA-binding sequence comprises RNA bases and DNA bases (e.g., the DNA-binding sequence comprises at least one thymine and at least one uracil).

In some instances, the RNA guide disclosed herein may further comprise a linker sequence, a 5′ end and/or 3′ end protection fragment (see disclosures herein), or a combination thereof.

The spacer in any of the RNA guides disclosed herein can be specific to a target sequence, i.e., capable of binding to the complementary region of the target sequence via base-pairing. In some instances, the target sequence may be within a genomic site of interest, e.g., where gene editing is needed.

In some embodiments, the target sequence is adjacent to a PAM sequence. PAM sequences are known in the art. In some embodiments, PAM sequences capable of being recognized by a CRISPR nuclease are disclosed in WO2021055874, WO2020206036, WO2020191102, WO2020186213, WO2020028555, WO2020033601, WO2019126762, WO2019126774, WO2019071048, WO2019018423, WO2019005866, WO2018191388, WO2018170333, WO2018035388, WO2018035387, WO2017219027, WO2017189308, WO2017184768, WO2017106657, WO2016205749, WO2017070605, WO2016205764, WO2016205711, WO2016028682, WO2015089473, WO2014093595, WO2015089427, WO2014204725, WO2015070083, WO2014093655, WO2014093694, WO2014093712, WO2014093635, WO2021133829, WO2021007177, WO2020197934, WO2020181102, WO2020181101, WO2020041456, WO2020023529, WO2020005980, WO2019104058, WO2019089820, WO2019089808, WO2019089804, WO2019089796, WO2019036185, WO2018226855, WO2018213351, WO2018089664, WO2018064371, WO2018064352, WO2017106569, WO2017048969, WO2016196655, WO2016106239, WO2016036754, WO2015103153, WO2015089277, WO2014150624, WO2013176772, WO2021119563, WO2021118626, WO2020247883, WO2020247882, WO2020223634, WO2020142754, WO2020086475, WO2020028729, WO2019241452, WO2019173248, WO2018236548, WO2018183403, WO2017027423, WO2018106727, WO2018071672, WO2017096328, WO2017070598, WO2016201155, WO2014150624, WO2013098244, WO2021113522, WO2021050534, WO2021046442, WO2021041569, WO2021007563, WO2020252378, WO2020180699, WO2020018142, WO2019222555, WO2019178428, WO2019178427, or WO2019006471, the relevant disclosures of each of which are incorporated for the subject matter and purpose referenced herein.

When the gene editing system comprises a Cas12i polypeptide, the PAM sequence comprises 5′-NTTN-3′ (or 5′-TTN-3′) wherein N is any nucleotide (e.g., A, G, T, or C). The PAM sequence is upstream to the target sequence. The PAM sequence in association with other CRISPR nucleases may comprises the sequence 5′-TTY-3′ or 5′-TTB-3′, wherein Y is C or T, and B is G, T, or C. The PAM sequence may be immediately adjacent to the target sequence or, for example, within a small number (e.g., 1, 2, 3, 4, or 5) of nucleotides of the target sequence.

When the gene editing system comprises the other Type V nuclease as disclosed herein, the PAM comprises the motif of 5′-NTTR-3′, in which N is any of A, T, G, and C; and R is A or G. The PAM motif is located 5′ to the target sequence.

The PAM sequences for Nucleases A-D are provided in Tables 3-5.

In some embodiments, the gRNA for the Cas12i2 (SEQ ID NO: 3 or SEQ ID NO: 266) nuclease may comprise the same spacer sequence as any of the gRNAs provided in Table 2 below. In one example, the gRNA may comprise the same spacer sequence as gRNA G53 (CUACCUUUCUCUCGAAGGUC; SEQ ID NO: 267). In another example, the gRNA may comprise the same spacer sequence as gRNA G55 (CUCUCGAAGGUCUUCUGCCG; SEQ ID NO: 268). In yet another example, the gRNA may comprise the same spacer sequence as gRNA G56 (UCUCGAAGGUCUUCUGCCGA; SEQ ID NO: 269). Exemplary gRNAs for the Cas12i2 (e.g., SEQ ID NO: 3 or SEQ ID NO: 266) nuclease are provided in Table 2 below, all of which are within the scope of the present disclosure.

In some embodiments, the gRNA for the Nucleases A-D may comprise the same spacer sequence as the gRNAs provided in Tables 3-5 below. In one example, the gRNA may comprise the same spacer sequence as gRNA A_STMN2_Splice2a_4 (UAUUCAUAUUGCAGGACUCG; SEQ ID NO: 270). In another example, the gRNA may comprise the same spacer sequence as gRNA A_STMN2_Splice2a_3 (AAAUUAUAUUCAUAUUGCAG; SEQ ID NO: 271). Exemplary gRNAs for the Nucleases A-D are provided in Tables 3-5 below, all of which are within the scope of the present disclosure.

### Modification of Nucleic Acids

Any of the gRNAs in the gene editing systems disclosed herein may include one or more modifications.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The gRNAs may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4 (1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27:196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyluridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, any gRNAs described herein may comprise an end modification (e.g., a 5′ end modification or a 3′ end modification). In some embodiments, the end modification is a chemical modification. In some embodiments, the end modification is a structural modification. See disclosures herein. In specific examples, the gRNAs may comprise 2′-o-methylation and phosphorothioate linkages, for example, at the 5′ and/or the 3′ end.

(iii) Delivery of Gene Editing System to Cells

In some embodiments, any of the gene editing systems or components thereof as disclosed herein may be formulated, for example, including a carrier, such as a carrier and/or a polymeric carrier, e.g., a liposome or lipid nanoparticle, and delivered by known methods to a cell (e.g., a neuron cell.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Type V CRISPR nuclease and/or one or more gRNAs), one or more transcripts thereof, and/or a pre-formed ribonucleoprotein to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, the present application further provides cells produced by such methods, and organisms (such as animals, plants, or fungi) comprising or produced from such cells. In some embodiments, a composition of the present invention is further delivered with an agent (e.g., compound, molecule, or biomolecule) that affects DNA repair or DNA repair machinery. In some embodiments, a composition of the present invention is further delivered with an agent (e.g., compound, molecule, or biomolecule) that affects the cell cycle.

In some embodiments, a first composition comprising a Type V CRISPR nuclease is delivered to a cell. In some embodiments, a second composition comprising a gRNA guide is delivered to a cell. In some embodiments, the first composition is contacted with a cell before the second composition is contacted with the cell. In some embodiments, the first composition is contacted with a cell at the same time as the second composition is contacted with the cell. In some embodiments, the first composition is contacted with a cell after the second composition is contacted with the cell. In some embodiments, the first composition is delivered by a first delivery method and the second composition is delivered by a second delivery method. In some embodiments, the first delivery method is the same as the second delivery method. For example, in some embodiments, the first composition and the second composition are delivered via viral delivery. In some embodiments, the first delivery method is different than the second delivery method. For example, in some embodiments, the first composition is delivered by viral delivery and the second composition is delivered by lipid nanoparticle-mediated transfer and the second composition is delivered by viral delivery or the first composition is delivered by lipid nanoparticle-mediated transfer and the second composition is delivered by viral delivery.

Alternatively, the components of the gene editing system provided herein (e.g., the Type V nuclease or its encoding nucleic acid and the guide RNA or its encoding nucleic acid) may be formulated in one composition, which can be delivered to host cells of interest. For example, the gene editing system may comprise a messenger RNA encoding the Type V nuclease and a gRNA, which can be formulated with lipid excipients and be delivered to host cells via lipid nanoparticle-mediated transfer. In another example, the gene editing system may comprise the Type V nuclease and the gRNA, which may form a ribonucleoprotein (RNP) complex. The RNP complex can be delivered to host cells via a suitable route as known in the art. As another example, the gene editing system may comprise an expression vector capable of producing both the Type V nuclease and the gRNA. Such an expression vector produces the nuclease and the gRNA when transferred into host cells. See above descriptions for such expression vectors.

### III. Therapeutic Applications

Any of the gene editing systems or modified cells (e.g., modified neuron cells) generated using such a gene editing system as disclosed herein may be used for treating a disease involving the aberrant splicing of STMN2 as disclosed herein, e.g., ALS or FTD.

Amyotrophic lateral sclerosis, or ALS, is a progressive nervous system disease that affects nerve cells in the brain and spinal cord, causing loss of muscle control. ALS often begins with muscle twitching and weakness in a limb, or slurred speech. Eventually, ALS affects control of the muscles needed to move, speak, eat and breathe. Currently, there is no cure for this fatal disease. Signs and symptoms of ALS vary greatly from person to person, depending on which neurons are affected. It generally begins with muscle weakness that spreads and gets worse over time. Exemplary signs and symptoms for ALS include: difficulty walking or doing normal daily activities, tripping and falling, weakness in your legs, feet or ankles, hand weakness or clumsiness, slurred speech or trouble swallowing, muscle cramps and twitching in arms, shoulders and tongue, inappropriate crying, laughing or yawning, and/or cognitive and behavioral changes.

Frontotemporal dementia (FTD), a common cause of dementia, is a group of disorders that occur when nerve cells in the frontal and temporal lobes of the brain are lost. This causes the lobes to shrink. FTD can affect behavior, personality, language, and movement. The most common types of FTD include frontal variant (affects behavior and personality), primary progressive aphasia (difficulty communicating), including progressive nonfluent aphasia (which affects the ability to speak) and semantic dementia (which affects the ability to use and understand language). A less common form of FTD affects movement, causing symptoms similar to Parkinson disease or amyotrophic lateral sclerosis.

In some embodiments, provided herein is a method for treating a target disease as disclosed herein (e.g., ALS or FTD) comprising administering to a subject (e.g., a human patient) in need of the treatment any of the gene editing systems disclosed herein. The gene editing system may be delivered to a specific tissue (e.g., brain) or specific type of cells (e.g., neuron cells) where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise the desired genetic editing in the STMN2 gene, including deletions in the desired location(s) (the 3′ splice site and/or the downstream 10-nt window) as disclosed herein described herein. The modified cells may be prepared using neuron cells isolated from a human patient (e.g., an ALS patient or a FTD patient). The modified cells thus prepared produce reduced levels of the non-functional STMN2 transcript due to the aberrant splicing event and increased levels of functional STMN2 transcripts (and thus functional STMN proteins) as compared with the non-edited counterparts.

In some embodiments, provided herein is a composition comprising the gene editing system disclosed herein or components thereof (e.g., the Cas12i2 polypeptide or the Type V CRISPR nucleases disclosed herein and the corresponding gRNA targeting the genomic site of interest in the STMN2 gene as also disclosed herein). Such a composition can be a pharmaceutical composition. A pharmaceutical composition that is useful may be prepared, packaged, or sold in a formulation for a suitable delivery route, e.g., parenteral, intra-lesional, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition (e.g., the gene editing system or components thereof), which would be administered to a subject or a convenient fraction of such a dosage such as, for example, one-half or one-third of such a dosage.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

In some instances, the gene editing system for the therapeutic applications disclosed herein may comprise a viral vector such as an AAV vector that comprises a coding sequence for the Type V nuclease disclosed herein. Such an AAV vector (e.g., AAVrh10) can be used to deliver the nuclease to a subject in need of the treatment (e.g., to a specific type of cells such as neuron cells where editing of the STMN2 gene is needed). In some examples, the AAV vector may also carry a coding sequence for the RNA guide so that the RNA guide can be produced in the host cells. Alternatively, the RNA guide may be co-delivered with the AAV vector to the subject in need of the treatment.

### IV. Kits for Inhibiting STMN2 Aberrant Splicing and Alleviating Target Diseases

The present disclosure also provides kits that can be used, for example, to carry out a gene editing method described herein for genetical modification of the STMN2 gene as disclosed herein. In some embodiments, the kits include an RNA guide and a Type V nuclease (e.g., a Cas12i polypeptide or another Type V nuclease as provided herein, e.g., in Table 1). In some embodiments, the kits include an RNA guide and a Type V nuclease. In other embodiments, the kits include a polynucleotide that encodes such a Type V nuclease, and optionally the polynucleotide is comprised within a vector, e.g., as described herein. In some embodiments, the kits include a polynucleotide that encodes an RNA guide disclosed herein. The Type V nuclease (or polynucleotide encoding the Type V nuclease) and the RNA guide (e.g., as a ribonucleoprotein) can be packaged within the same or other vessel within a kit or can be packaged in separate vials or other vessels, the contents of which can be mixed prior to use.

The Type V nuclease and the RNA guide can be packaged within the same or other vessel within a kit or can be packaged in separate vials or other vessels, the contents of which can be mixed prior to use. The kits can additionally include, optionally, a buffer and/or instructions for use of the RNA guide and Type V nuclease.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1—Targeting STMN2 Exon 2a Splice Site by Cas12i2 in SH-SY5Y Cells

This Example shows that using Cas12i2 complexed with RNA guides designed to disrupt the exon 2a splice site of STMN2 (the 3′ splice site in intron 1 as disclosed herein) results in a reduction of the STMN2 exon 2a splice variant and a corresponding increase in full length STMN2 transcripts.

SH-SY5Y cells were cultured for 48 hours, to a confluency of 70-80% in DMEM/F12 (Gibco #10565018) containing 10% FBS (Hyclone, Heat inactivated #SH30071.03). Cells were lifted with TrypLE (Gibco), counted, rinsed with PBS, and resuspended in Lonza SF nucleofection buffer+supplement (Lonza, V4XC-2024) at a concentration of 20,000 cells/uL. Two million cells were used per electroporation reaction.

Each RNA guide of Table 2 designed to target a 3′ splice site of STMN2 intron 1 (the splice site for exon 2a) was complexed with a variant Cas12i2 (SEQ ID NO: 3). Variant Cas12i2/RNA guide RNP complexes were generated by mixing Cas12i2 (in 50 mM HEPES, 700 mM NaCl, 0.5 mM TECEP (tris(2-carboxyethyl) phosphine), 5% Glycerol, pH 7.5) with RNA guides (in 250 mM NaCl) on ice at a 1:2.5 molar ratio for 60 minutes. RNPs were added to each reaction at a final concentration of 20 μM (Cas12i2) and 50 μM RNA guide, in the presence of 1 μM siTARDBP (siTDP-43 RNA; Horizon Discovery Biosciences ON-TARGETplus Human TARDP [GCUCAAGCAUGGAUUCUAA (SEQ ID NO:7), CAAUCAAGGUAGUAAUAUG (SEQ ID NO:8), GGGCUUCGCUACAGGAAUC (SEQ ID NO: 9); and CAGGGUGGAUUUGGUAAUA (SEQ ID NO:10)]) or siNon-targeting Pool (siCont RNA; Horizon Discovery Biosciences ON-TARGETplus Non-targeting Pool).

TABLE 2
Cas12i2 RNA Guides Targeting Human Stathmin-2

| Guide | Sequence* | SEQ ID |
|---|---|---|
| STMN2-Exon2a-1(G1) | AGAAAUCCGUCUUUCAUUGACGGUGCCCCAUCACUCUCUCUUA | 11 |
| STMN2-Exon2a-2(G2) | AGAAAUCCGUCUUUCAUUGACGGAUUGGAUUUUUAAAAUUAUA | 12 |
| STMN2-Exon2a-3(G3) | AGAAAUCCGUCUUUCAUUGACGGGAUUUUUAAAAUUAUAUUCA | 13 |
| STMN2-Exon2a-4(G4) | AGAAAUCCGUCUUUCAUUGACGGUUAAAAUUAUAUUCAUAUUG | 14 |
| STMN2-Exon2a-5(G5) | AGAAAUCCGUCUUUCAUUGACGGUAAAAUUAUAUUCAUAUUGC | 15 |
| STMN2-Exon2a-6(G6) | AGAAAUCCGUCUUUCAUUGACGGAAAAUUAUAUUCAUAUUGCA | 16 |
| STMN2-Exon2a-7(G7) | AGAAAUCCGUCUUUCAUUGACGGAAAUUAUAUUCAUAUUGCAG | 17 |
| STMN2-Exon2a-8(G8) | AGAAAUCCGUCUUUCAUUGACGGUAUUCAUAUUGCAGGACUCG | 18 |
| STMN2-Exon2a-9(G9) | AGAAAUCCGUCUUUCAUUGACGGAUAUUGCAGGACUCGGCAGA | 19 |
| STMN2-Exon2a-10(G10) | AGAAAUCCGUCUUUCAUUGACGGCAGGACUCGGCAGAAGACCU | 20 |
| STMN2-Exon2a-11(G11) | AGAAAUCCGUCUUUCAUUGACGGGAGAGAAAGGUAGAAAAUAA | 21 |
| STMN2-Exon2a-12(G12) | AGAAAUCCGUCUUUCAUUGACGGGGCUCUCUGUGUGAGCAUGU | 22 |
| STMN2-Exon2a-17(G17) | AGAAAUCCGUCUUUCAUUGACGGAUGAAUAGCAAUACUGAAGA | 23 |
| STMN2-Exon2a-24(G24) | AGAAAUCCGUCUUUCAUUGACGGGGUAAAUAAUAAAUAUAAGA | 24 |
| STMN2-Exon2a-48(G48) | AGAAAUCCGUCUUUCAUUGACGGUUAGGCAGGCUGUCUGUCUC | 25 |
| STMN2-Exon2a-50(G50) | AGAAAUCCGUCUUUCAUUGACGGUUAUUUUCUACCUUUCUCUC | 26 |
| STMN2-Exon2a-51(G51) | AGAAAUCCGUCUUUCAUUGACGGUUUUCUACCUUUCUCUCGAA | 27 |
| STMN2-Exon2a-52(G52) | AGAAAUCCGUCUUUCAUUGACGGUCUACCUUUCUCUCGAAGGU | 28 |
| STMN2-Exon2a-53(G53) | AGAAAUCCGUCUUUCAUUGACGGCUACCUUUCUCUCGAAGGUC | 29 |
| STMN2-Exon2a-54(G54) | AGAAAUCCGUCUUUCAUUGACGGUACCUUUCUCUCGAAGGUCU | 30 |
| STMN2-Exon2a-55(G55) | AGAAAUCCGUCUUUCAUUGACGGCUCUCGAAGGUCUUCUGCCG | 31 |
| STMN2-Exon2a-56(G56) | AGAAAUCCGUCUUUCAUUGACGGUCUCGAAGGUCUUCUGCCGA | 32 |
| STMN2-Exon2a-57(G57) | AGAAAUCCGUCUUUCAUUGACGGUGCCGAGUCCUGCAAUAUGA | 33 |
| STMN2-Exon2a-58(G58) | AGAAAUCCGUCUUUCAUUGACGGUAAAAAUCCAAUUAAGAGAG | 34 |
| Cas12i2_non- | AGAAAUCCGUCUUUCAUUGACGGAGUGCGUACGAGCUCGGACG | 35 |
| target_negcontol | | |

*spacer sequence in boldface.

The nuclease-binding fragment in the exemplary gRNAs for Cas12i2 has the nucleotide sequence of AGAAAUCCGUCUUUCAUUGACGG (SEQ ID NO: 36).

The cuvettes were electroporated using an electroporation device (program CA-137, Lonza 4D-nucleofector). Following electroporation, cells were allowed to rest for 10 minutes before being added to pre-warmed culture medium and mixed gently by pipetting. 90% of cells were plated for downstream RNA analysis, and 10% were for NGS. The cells were then incubated at 37° C. for 72 hours. Next, cells were rinsed with PBS and lysed with QuickExtract™ (DNA extraction solution; Lucigen) or RLT+ (Qiagen). Cells in QuickExtract were incubated at 65° C. for 15 minutes, 68° C. for 15 minutes, and 98° C. for 10 minutes, before being analyzed by NGS. Samples for NGS were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were done with a 300 Cycle NEXTSEQ™ (Illumina) 500/550 High Output v2.5 Kit.

For cells in RLT+, RNA isolation was performed using QIAshredder columns and RNeasy plus Micro Kit (Qiagen). RNA (2 μg) was converted to cDNA using High-Capacity cDNA Reverse Transcription kit (Applied Biosystems). Quantitative reverse transcription PCR was performed using Faststart Universal Probe Master (Rox), (Roche), and TaqMan probes for TARDP (Hs00606522_m1, Thermo Fisher), STMN2 (Hs00975900_m1, Thermo Fisher) and GAPDH (Hs02786624_g1, Thermo Fisher). A TaqMan probe and PCR primers were used for detecting the STMN2 exon 2a splice variant.

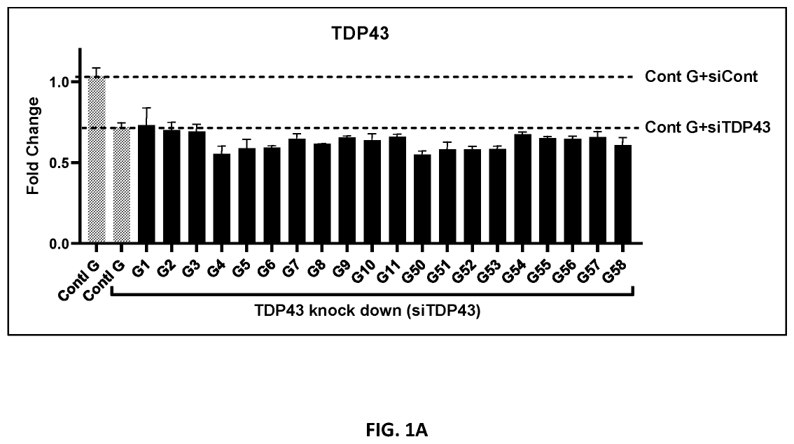

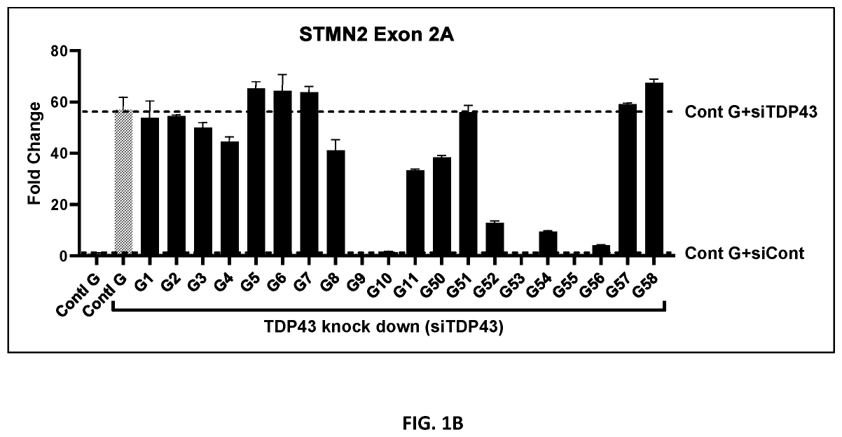

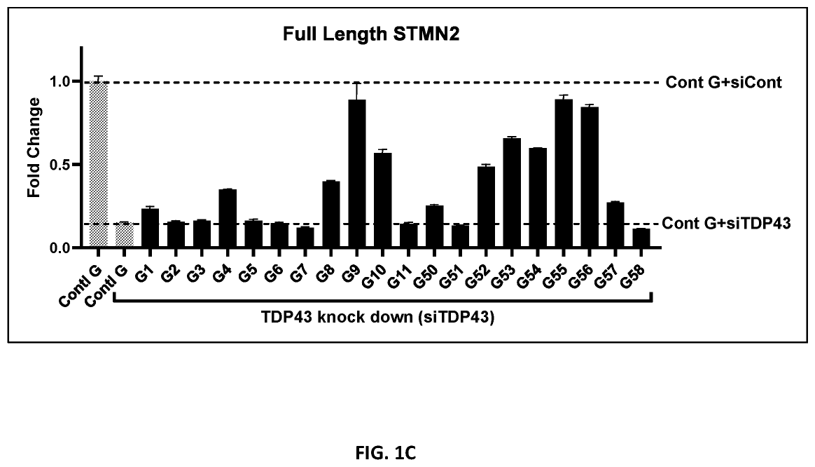

As shown in FIG. 1A, a 30-fold TDP-43 knockdown was observed in SHSY5Y cells using siTDP-43 RNA. Knockdown of TDP-43 in the presence of a non-targeting control RNP complex resulted in a 60-fold increase in the STMN2 exon 2a splice variant and in over a 90-fold decrease in the STMN2 full length transcript (FIG. 1B and FIG. 1C). The exon 2a increase was partially or totally mitigated by RNP guides G9, G10, G11, G50, G52, G53, G54, G55, G56, G48 and G17 (FIG. 1B). An inverse relationship was observed between exon 2a and full length Stathmin-2 RNA (FIG. 1C). Significant restoration of the decreased full length STMN2 signal, due to TDP-43 depletion, was observed with guides G8, G9, G10, G52, G53, G54, G55 and G56. Guides G9, G55 and G56 showed the highest fold-changes for both the full-length transcript and the aberrantly spliced transcript.

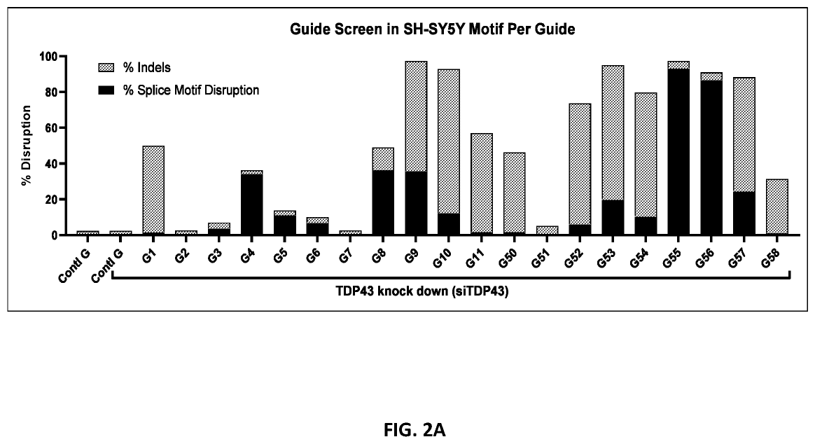

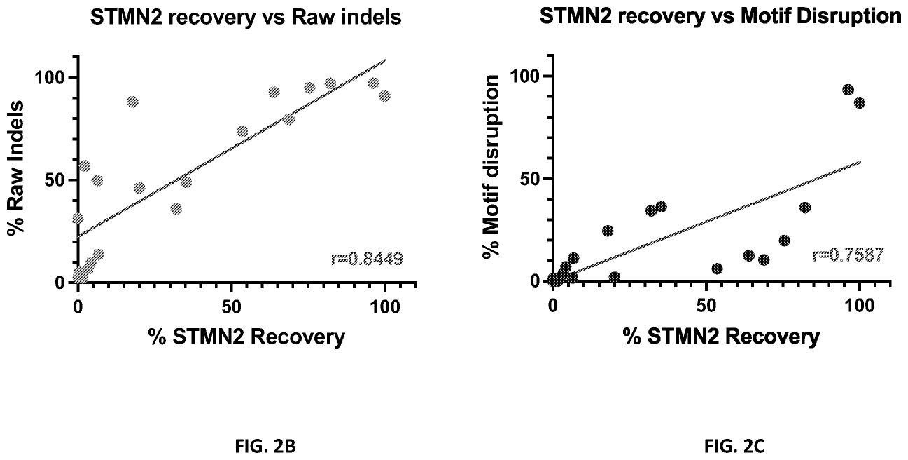

FIGS. 2A-2C show indel activity and splice region disruption of the tested RNA guides in SH-SY5Y cells. It was observed a correlation between guides that promoted STMN2 full length recovery and motif disruption (FIG. 2C), as well as raw indels and STMN2 full length recovery (FIG. 2B). Guide 9 showed around 36% splice site motif disruption and 97.2% overall editing; greater than 35% of total edits disrupted the splice site (FIG. 2A). Guides G55 and G56 showed the highest indels and resulted in the highest motif disruption among all tested guides (FIG. 2A). Guide G55 showed 93.5% splice site motif disruption and 97.3% overall editing; greater than 95% of total edits disrupted the splice site. Guide G56 showed around 87% splice site motif disruption and 91% overall editing; greater than 95% of total edits disrupted the splice site.

Therefore, this Example shows that in the presence of TDP-43 knockdown, editing with Cas12i2 and RNA guides targeting the STMN2 exon 2a splice site not only disrupts the DNA splice site, resulting in a decrease in the exon 2a splice variant, but also promotes a corresponding increase in full length STMN2.

### Example 2—Targeting STMN2 Exon 2a Splice Site and TDP-43 Binding Site by Cas12i2 in SH-SY5Y Cells

This Example shows that an RNA guide designed to disrupt the TDP-43 binding site recapitulates the effects of TDP-43 knockdown by siRNA, demonstrating the effectiveness of RNA guide disruption of the STMN2 exon 2a site.

SH-SY5Y cells were cultured for 48 hours to a confluency of 70-80% in DMEM/F12 (Gibco #10565018) containing 10% FBS (Hyclone, Heat inactivated #SH30071.03). Cells were lifted with TrypLE (Gibco), counted, rinsed with PBS, and resuspended in Lonza SF nucleofection buffer+supplement (Lonza, V4XC-2024) at a concentration of 20,000 cells/μL.

Two million cells were co-nucleofected with RNP complexes containing RNA guides targeting the TDP-43 binding site

(Guide 12: AGAAAUCCGUCUUUCAUUGACGGGGC
UCUCUGUGUGAGCAUGU; SEQ ID NO: 39),

the STMN2 exon 2a splice site

(Guide 55: AGAAAUCCGUCUUUCAUUGACGGCUC
UCGAAGGUCUUCUGCCG; SEQ ID NO: 40),

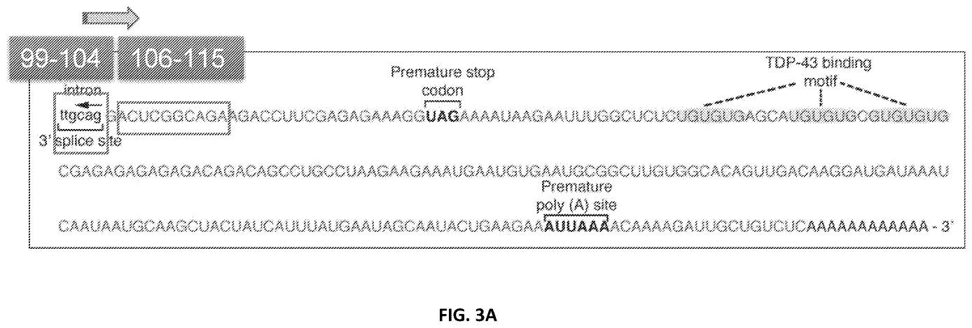

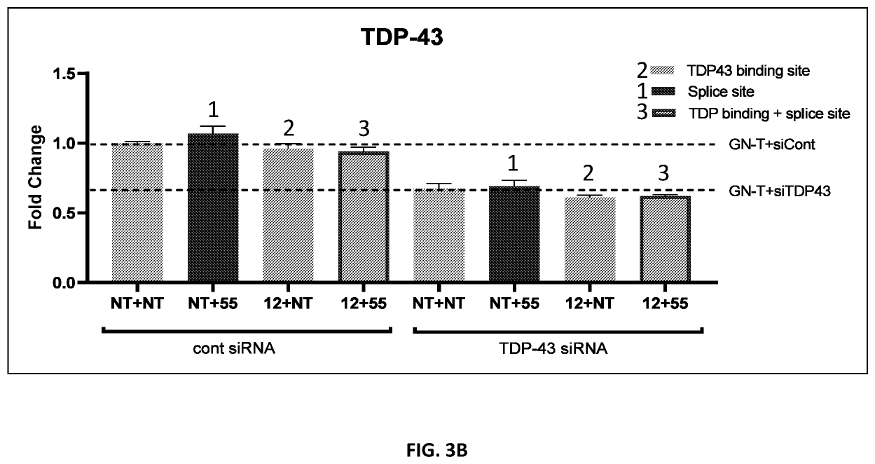

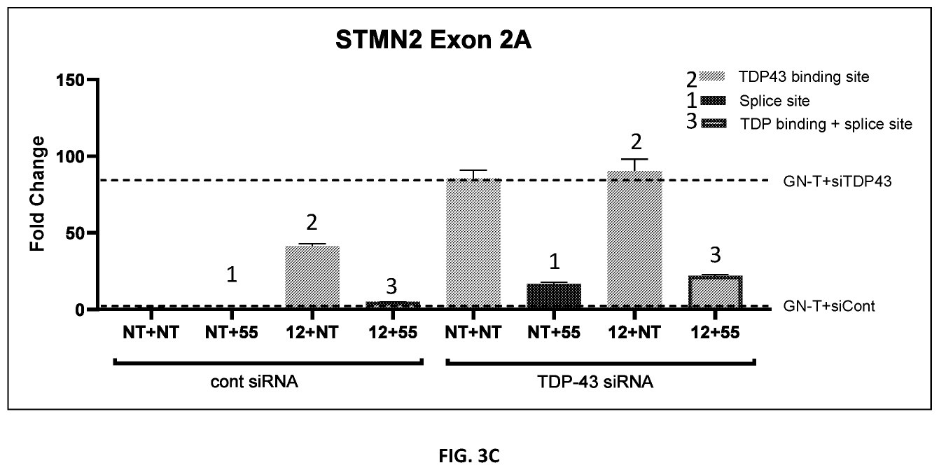

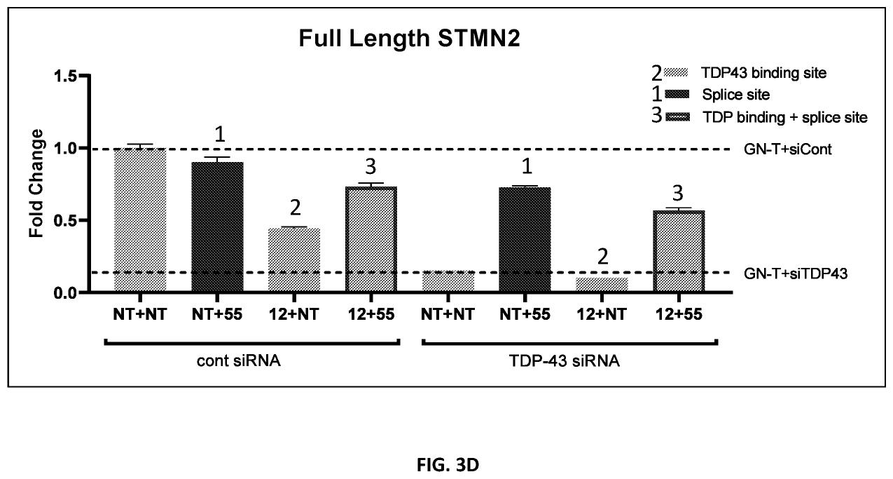

or a non-targeting guide (AGAAAUCCGUCUUUCAUUGACGGAGUGCGUACGAGCUCGGACG; SEQ ID NO: 41), both in the presence and absence of siRNA mediated TDP-43 knockdown, as described in Example 1. See FIG. 3A for positions of the 3′ splice site, the 10-nt window, and the TDP-43 binding motif in the corresponding region of the STMN2 gene.

FIG. 3B shows that disruption of the TDP-43 binding site with RNA guide 12 recapitulates the effect of siRNA TDP-43 knockdown. In the absence of siRNA mediated TDP-43 knockdown and presence of guide 12, the downstream effect of splice site disruption with guide G55—a decrease in the exon 2a splice variant and an increase in full length STMN2—is also observed (FIG. 3C and FIG. 3D, respectively).

This Example thus shows that co-nucleofection of Cas12i2 RNPs targeting the TDP-43 binding site and the exon 2a splice site results in STMN2 exon 2a decrease and full length STMN2 increase, with or without siRNA-induced TDP-43 knockdown. These results demonstrate the feasibility of an in vivo strategy using mice harboring a humanized STMN2 gene with a constitutively disrupted TDP-43 binding site. Such a strategy enables investigation of the in vivo effects of STMN2 exon 2a splice site disruption by RNA guides disclosed herein without the need to knockdown TDP-43 in the animals, as knocking down TDP-43 in vivo could result in non-STMN2 related outcomes.

### Example 3—Computational Analysis of Indels Induced by Cas12i2

In this Example, a computational analysis was performed on samples from Example 1 to determine how indels within or near the 3′ splice site of exon 2a correlate with full-length STMN2 recovery. The full-length STMN2 transcript refers to the mRNA containing exons 1-5, which encodes a functional STMN2 protein.

To identify the optimal disruption region, a sliding window of variable size (1-10-nt) was used to calculate the positional indel rates from the nucleotides within the window. This was performed for each guide tested (n=20) across the STMN2 amplicon sequence. The correlations between window positional indel rate and STMN2 recovery rate were calculated for each position and window size to determine the amplicon position and window size with the maximum correlation coefficient, indicating which disruption site/motif maximizes STMN2 transcript recovery.

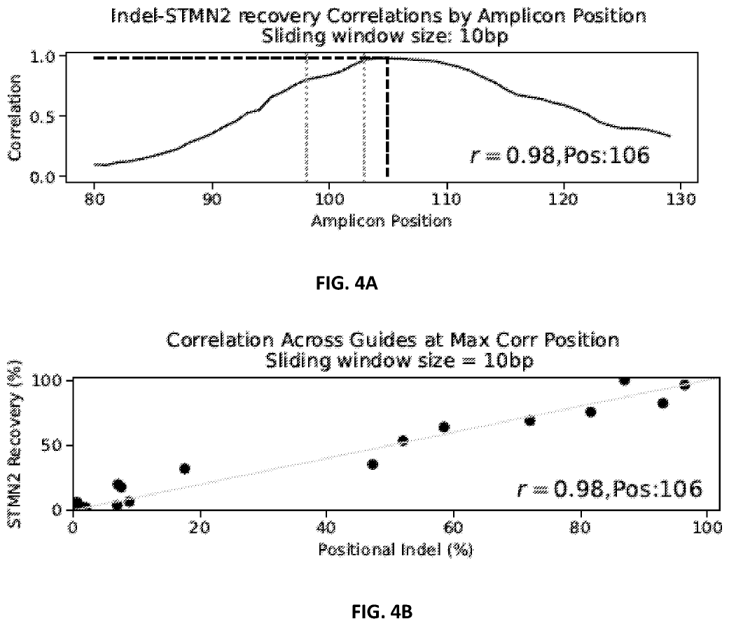

FIGS. 4A-4B show representative plots for one dataset analyzed, showing the maximum correlation observed with a 10-nt window starting at amplicon position 106. FIG. 4A shows correlation values between position indel rates and STMN2 recovery rates across guides (y-axis) for all amplicon positions (x-axis) are plotted with the position for maximum correlation (black dotted line). FIG. 4B shows scatter plot of guide position indels rates with a 10-nt window starting at position 106 (x-axis) and STMN2 recovery rates (y-axis).

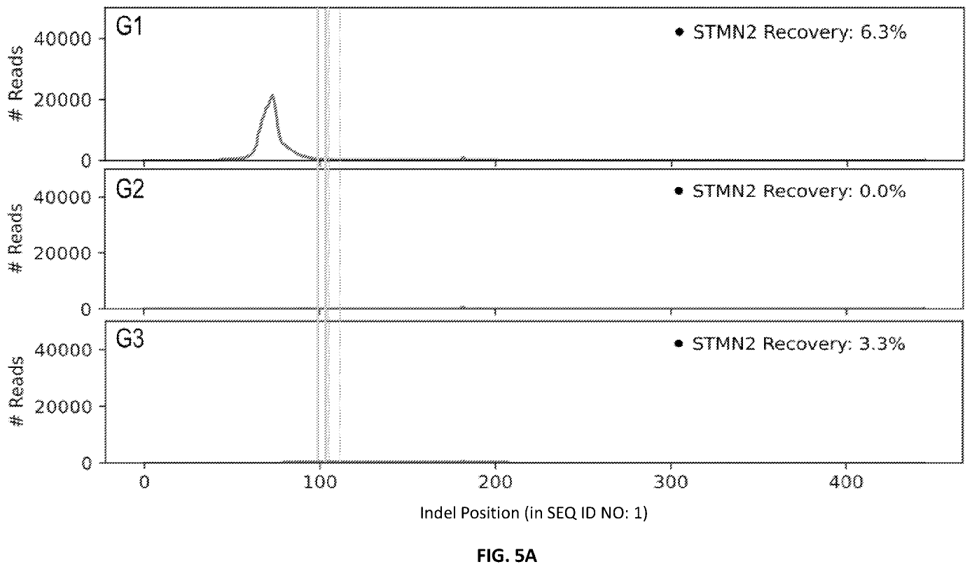

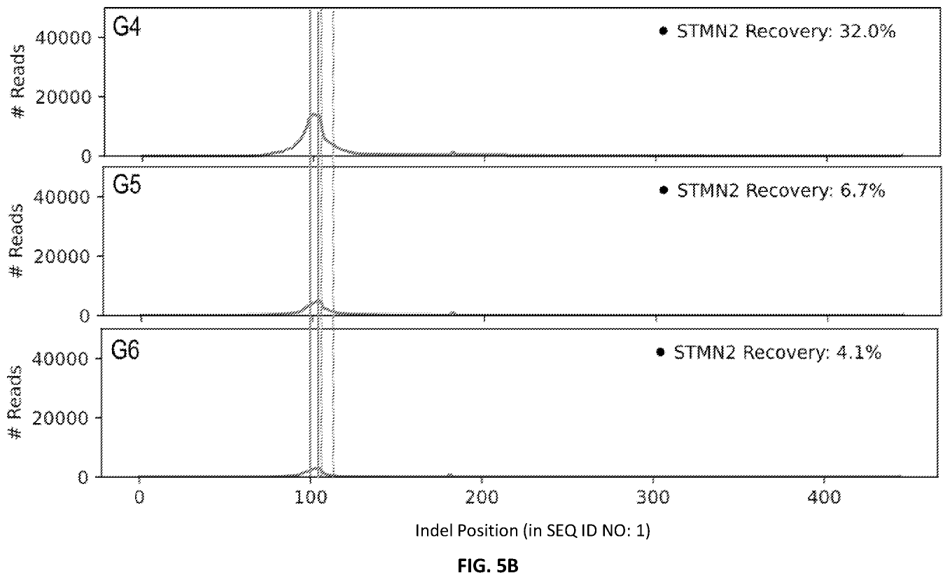

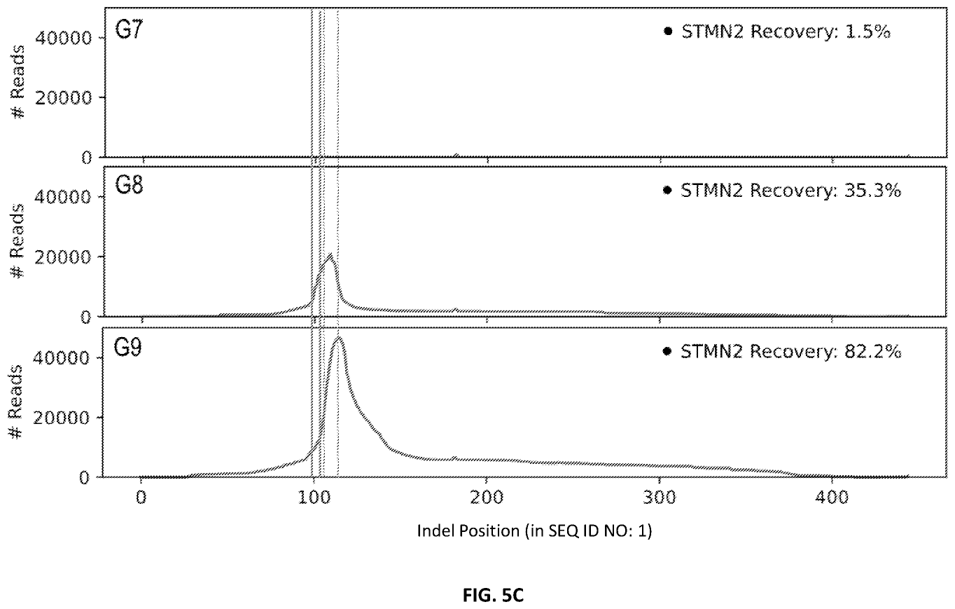

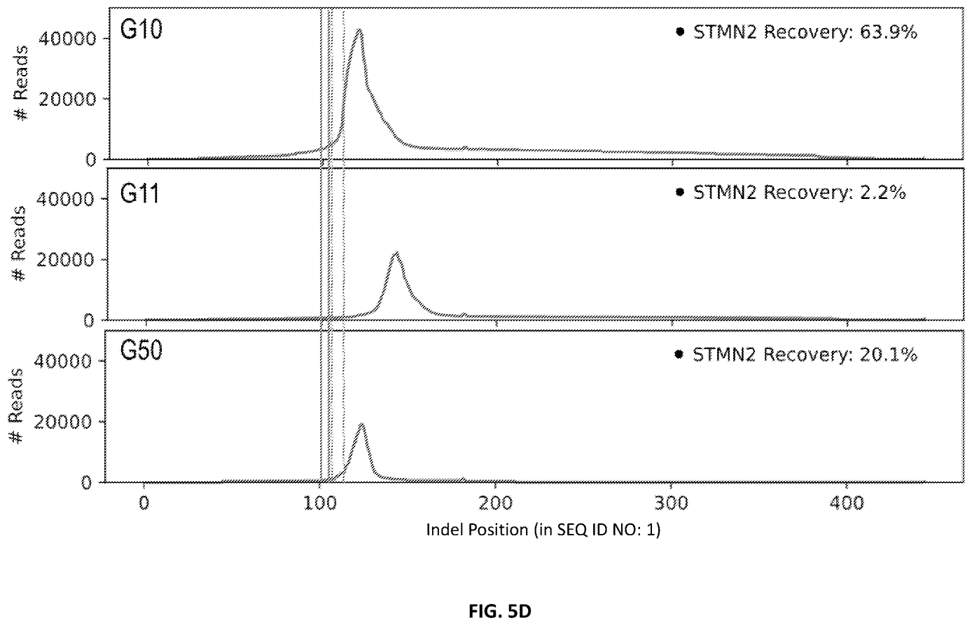

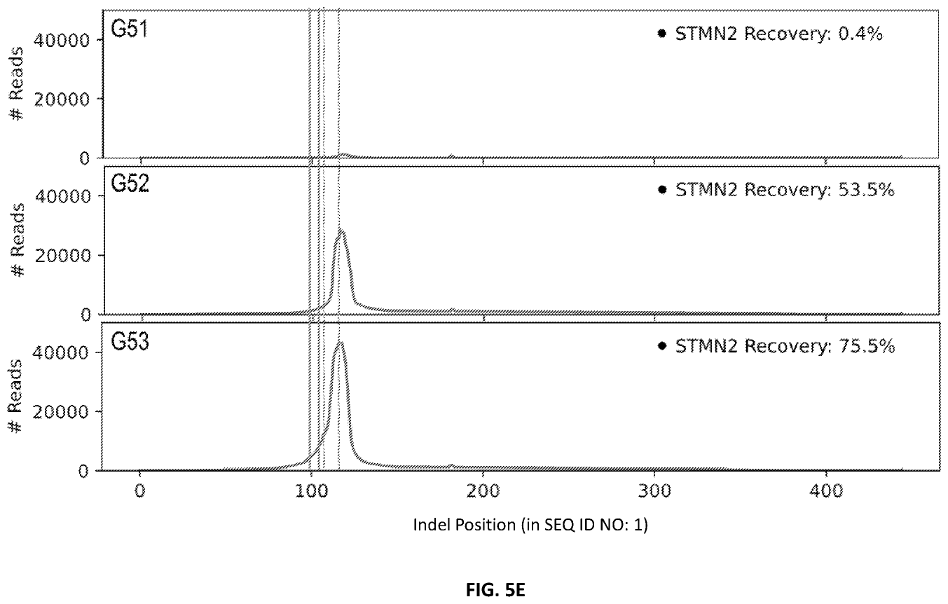

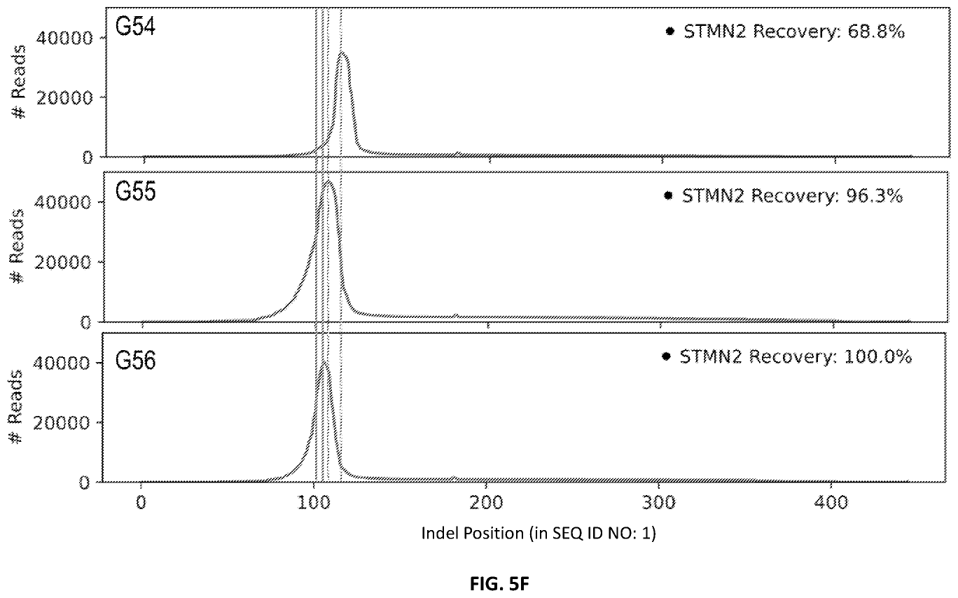

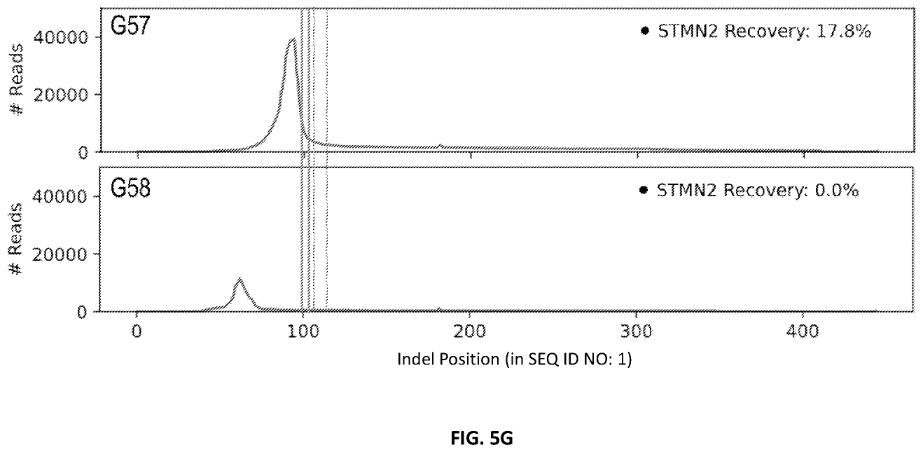

FIGS. 5A-5G show the positions of indels induced by a particular guide within the STMN2 amplicon (x-axis). The number of NGS reads is on the y-axis. The region defined by the solid lines on the left indicates the position of the literature-defined splice site. The region defined by the dashed lines on the right indicates the position of the optimal disruption window identified above. Minimal or no full-length STMN2 recovery was observed for guides that did not induce indels within either window. See, e.g., RNA guides G1, G2, G3, G7, and G11 in FIGS. 5A, 5C, and 5D. Measurable full-length STMN2 recovery was observed with guides inducing indels primarily within the literature-defined splice site (see, e.g., RNA guide G4 in FIG. 5B) or primarily within the optimal disruption window defined herein (see, e.g., guides G9, G10, G53, and G54, in FIGS. 5C, 5D, 5E, and 5F). The greatest full-length STMN2 recovery was observed with guides inducing indels within the 3′ splice site and the optimal disruption window identified herein (see, e.g., RNA guides G55 and G56 in FIG. 5F).

These results indicate that disruption of nucleotides within a 10-nt window downstream of the STMN2 3′ splice site provides a high recovery rate of full-length STMN2.

### Example 4—Editing of STMN2 with Type V CRISPR Nucleases

In this Example, RNA guides were designed to target the optimal disruption window of exon 2a of STMN2 identified in Example 3 above. Indels were assessed in cells after transfection with plasmids coding for a Type V nuclease in Table 1 above and an RNA guide in Tables 3-5 below.

TABLE 3
RNA Guide and Target Sequences for Nuclease A

| Reference Name | PAM | Target Sequence | Guide Sequence* |
|---|---|---|---|
| A_STMN2_Splice | CTTA | ATTGGATTTTTAAAATTATA | CUUGUUGUAUAUGUCCUUUUAUAGG |
| 2a_1 | | (SEQ ID NO: 42) | UAUUAAACAACAUUGGAUUUUUAAA |
| | | | AUUAUA (SEQ ID NO: 49) |
| A STMN2_Splice | ATTG | GATTTTTAAAATTATATTCA | CUUGUUGUAUAUGUCCUUUUAUAGG |
| 2a_2 | | (SEQ ID NO: 43) | UAUUAAACAACGAUUUUUAAAAUUA |
| | | | UAUUCA (SEQ ID NO: 50) |
| A_STMN2_Splice | TTTA | AAATTATATTCATATTGCAG | CUUGUUGUAUAUGUCCUUUUAUAGG |
| 2a_3 | | (SEQ ID NO: 44) | UAUUAAACAACAAAUUAUAUUCAUA |
| | | | UUGCAG (SEQ ID NO: 51) |
| A_STMN2_Splice | ATTA | TATTCATATTGCAGGACTCG | CUUGUUGUAUAUGUCCUUUUAUAGG |
| 2a_4 | | (SEQ ID NO: 45) | UAUUAAACAACUAUUCAUAUUGCAG |
| | | | GACUCG (SEQ ID NO: 52) |
| A_STMN2_Splice | ATTG | CAGGACTCGGCAGAAGACCT | CUUGUUGUAUAUGUCCUUUUAUAGG |
| 2a_5 | | (SEQ ID NO: 46) | UAUUAAACAACCAGGACUCGGCAGA |
| | | | AGACCU (SEQ ID NO: 53) |
| A_STMN2_Splice | CTTA | TTTTCTACCTTTCTCTCGAA | CUUGUUGUAUAUGUCCUUUUAUAGG |
| 2a_6 | | (SEQ ID NO: 47) | UAUUAAACAACUUUUCUACCUUUCU |
| | | | CUCGAA (SEQ ID NO: 54) |
| A_STMN2_Splice | TTTA | AAAATCCAATTAAGAGAGAG | CUUGUUGUAUAUGUCCUUUUAUAGG |
| 2a_7 | | (SEQ ID NO: 48) | UAUUAAACAACAAAAUCCAAUUAAG |
| | | | AGAGAG (SEQ ID NO: 55) |

*The RNA guides for Nuclease A includes the nuclease-binding fragment of CUUGUUGUAUAUGUCCUUUUAUAGGUAUUAAACAAC (SEQ ID NO: 56). The spacer sequence in each guide RNA is underlined

TABLE 4
RNA Guide and Target Sequences for Nuclease B

| Reference | | | SEQ | | SEQ |
|---|---|---|---|---|---|
| Name | PAM | Target Sequence | ID NO | Guide Sequence* | ID NO |
| B_STMN2_ | CTTA | ATTGGATTTTTAAAATTATA | 57 | CCUGUUGUGAAUACUCUUUUAUAG | 120 |
| Splice2a_1 | | | | GUAUCAAACAACAUUGGAUUUUUA | |
| | | | | AAAUUAUA | |
| B_STMN2_ | TTTA | AAATTATATTCATATTGCAG | 58 | CCUGUUGUGAAUACUCUUUUAUAG | 121 |
| Splice2a_3 | | | | GUAUCAAACAACAAAUUAUAUUCA | |
| | | | | UAUUGCAG | |
| B_STMN2_ | ATTA | TATTCATATTGCAGGACTCG | 59 | CCUGUUGUGAAUACUCUUUUAUAG | 122 |
| Splice2a_4 | | | | GUAUCAAACAACUAUUCAUAUUGC | |
| | | | | AGGACUCG | |
| B_STMN2_ | ATTG | CAGGACTCGGCAGAAGACCT | 60 | CCUGUUGUGAAUACUCUUUUAUAG | 123 |
| Splice2a_5 | | | | GUAUCAAACAACCAGGACUCGGCA | |
| | | | | GAAGACCU | |
| B_STMN2_ | CTTA | TTTTCTACCTTTCTCTCGAA | 61 | CCUGUUGUGAAUACUCUUUUAUAG | 124 |
| Splice2a_6 | | | | GUAUCAAACAACUUUUCUACCUUU | |
| | | | | CUCUCGAA | |
| B_STMN2_ | TTTA | AAAATCCAATTAAGAGAGAG | 62 | CCUGUUGUGAAUACUCUUUUAUAG | 125 |
| Splice2a_7 | | | | GUAUCAAACAACAAAAUCCAAUUA | |
| | | | | AGAGAGAG | |
| B STMN2_ | G | CCCCATCACTCTCTCTTAAT | 63 | CCUGUUGUGAAUACUCUUUUAUAG | 126 |
| R_Splice2a_ | | | | GUAUCAAACAACCCCCAUCACUCU | |
| 1 | | | | CUCUUAAU | |
| B_STMN2_ | A | TCACTCTCTCTTAATTGGAT | 64 | CCUGUUGUGAAUACUCUUUUAUAG | 127 |
| R_Splice2a_ | | | | GUAUCAAACAACUCACUCUCUCUU | |
| 2 | | | | AAUUGGAU | |
| B_STMN2_ | A | CTCTCTCTTAATTGGATTTT | 65 | CCUGUUGUGAAUACUCUUUUAUAG | 128 |
| R_Splice2a_ | | | | GUAUCAAACAACCUCUCUCUUAAU | |
| 3 | | | | UGGAUUUU | |
| B_STMN2_ | A | ATTGGATTTTTAAAATTATA | 66 | CCUGUUGUGAAUACUCUUUUAUAG | 129 |
| R_Splice2a_ | | | | GUAUCAAACAACAUUGGAUUUUUA | |
| 4 | | | | AAAUUAUA | |
| B_STMN2_ | A | TTGGATTTTTAAAATTATAT | 67 | CCUGUUGUGAAUACUCUUUUAUAG | 130 |
| R_Splice2a_ | | | | GUAUCAAACAACUUGGAUUUUUAA | |
| 5 | | | | AAUUAUAU | |
| B_STMN2_ | A | TTTTTAAAATTATATTCATA | 68 | CCUGUUGUGAAUACUCUUUUAUAG | 131 |
| R_Splice2a_ | | | | GUAUCAAACAACUUUUUAAAAUUA | |
| 8 | | | | UAUUCAUA | |
| B_STMN2_ | A | AAATTATATTCATATTGCAG | 69 | CCUGUUGUGAAUACUCUUUUAUAG | 132 |
| R_Splice2a_ | | | | GUAUCAAACAACAAAUUAUAUUCA | |
| 9 | | | | UAUUGCAG | |
| B_STMN2_ | A | AATTATATTCATATTGCAGG | 70 | CCUGUUGUGAAUACUCUUUUAUAG | 133 |
| R_Splice2a_ | | | | GUAUCAAACAACAAUUAUAUUCAU | |
| 10 | | | | AUUGCAGG | |
| B_STMN2_ | A | ATTATATTCATATTGCAGGA | 71 | CCUGUUGUGAAUACUCUUUUAUAG | 134 |
| R_Splice2a_ | | | | GUAUCAAACAACAUUAUAUUCAUA | |
| 11 | | | | UUGCAGGA | |
| B_STMN2_ | A | TTATATTCATATTGCAGGAC | 72 | CCUGUUGUGAAUACUCUUUUAUAG | 135 |
| R_Splice2a_ | | | | GUAUCAAACAACUUAUAUUCAUAU | |
| 12 | | | | UGCAGGAC | |
| B_STMN2_ | A | TATTCATATTGCAGGACTCG | 73 | CCUGUUGUGAAUACUCUUUUAUAG | 136 |
| R_Splice2a_ | | | | GUAUCAAACAACUAUUCAUAUUGC | |
| 13 | | | | AGGACUCG | |
| B_STMN2_ | A | TTCATATTGCAGGACTCGGC | 74 | CCUGUUGUGAAUACUCUUUUAUAG | 137 |
| R_Splice2a_ | | | | GUAUCAAACAACUUCAUAUUGCAG | |
| 14 | | | | GACUCGGC | |
| B_STMN2_ | A | TATTGCAGGACTCGGCAGAA | 75 | CCUGUUGUGAAUACUCUUUUAUAG | 138 |
| R_Splice2a_ | | | | GUAUCAAACAACUAUUGCAGGACU | |
| 15 | | | | CGGCAGAA | |
| B_STMN2_ | A | TTGCAGGACTCGGCAGAAGA | 76 | CCUGUUGUGAAUACUCUUUUAUAG | 139 |
| R_Splice2a_ | | | | GUAUCAAACAACUUGCAGGACUCG | |
| 16 | | | | GCAGAAGA | |
| B_STMN2_ | G | CAGGACTCGGCAGAAGACCT | 77 | CCUGUUGUGAAUACUCUUUUAUAG | 140 |
| R_Splice2a_ | | | | GUAUCAAACAACCAGGACUCGGCA | |
| 17 | | | | GAAGACCU | |
| B_STMN2_ | A | GGACTCGGCAGAAGACCTTC | 78 | CCUGUUGUGAAUACUCUUUUAUAG | 141 |
| R_Splice2a_ | | | | GUAUCAAACAACGGACUCGGCAGA | |
| 18 | | | | AGACCUUC | |
| B_STMN2_ | G | GACTCGGCAGAAGACCTTCG | 79 | CCUGUUGUGAAUACUCUUUUAUAG | 142 |
| R_Splice2a_ | | | | GUAUCAAACAACGACUCGGCAGAA | |
| 19 | | | | GACCUUCG | |
| B_STMN2_ | G | ACTCGGCAGAAGACCTTCGA | 80 | CCUGUUGUGAAUACUCUUUUAUAG | 143 |
| R_Splice2a_ | | | | GUAUCAAACAACACUCGGCAGAAG | |
| 20 | | | | ACCUUCGA | |
| B_STMN2_ | A | CTCGGCAGAAGACCTTCGAG | 81 | CCUGUUGUGAAUACUCUUUUAUAG | 144 |
| R_Splice2a_ | | | | GUAUCAAACAACCUCGGCAGAAGA | |
| 21 | | | | CCUUCGAG | |
| B_STMN2_ | G | GCAGAAGACCTTCGAGAGAA | 82 | CCUGUUGUGAAUACUCUUUUAUAG | 145 |
| R_Splice2a_ | | | | GUAUCAAACAACGCAGAAGACCUU | |
| 22 | | | | CGAGAGAA | |
| B_STMN2_ | G | CAGAAGACCTTCGAGAGAAA | 83 | CCUGUUGUGAAUACUCUUUUAUAG | 146 |
| R_Splice2a_ | | | | GUAUCAAACAACCAGAAGACCUUC | |
| 23 | | | | GAGAGAAA | |
| B_STMN2_ | A | GAAGACCTTCGAGAGAAAGG | 84 | CCUGUUGUGAAUACUCUUUUAUAG | 147 |
| R_Splice2a_ | | | | GUAUCAAACAACGAAGACCUUCGA | |
| 24 | | | | GAGAAAGG | |
| B_STMN2_ | G | AAGACCTTCGAGAGAAAGGT | 85 | CCUGUUGUGAAUACUCUUUUAUAG | 148 |
| R_Splice2a_ | | | | GUAUCAAACAACAAGACCUUCGAG | |
| 25 | | | | AGAAAGGU | |
| B_STMN2_ | A | AGACCTTCGAGAGAAAGGTA | 86 | CCUGUUGUGAAUACUCUUUUAUAG | 149 |
| R_Splice2a_ | | | | GUAUCAAACAACAGACCUUCGAGA | |
| 26 | | | | GAAAGGUA | |
| B_STMN2_ | A | GACCTTCGAGAGAAAGGTAG | 87 | CCUGUUGUGAAUACUCUUUUAUAG | 150 |
| R_Splice2a_ | | | | GUAUCAAACAACGACCUUCGAGAG | |
| 27 | | | | AAAGGUAG | |
| B_STMN2_ | G | ACCTTCGAGAGAAAGGTAGA | 88 | CCUGUUGUGAAUACUCUUUUAUAG | 151 |
| R_Splice2a_ | | | | GUAUCAAACAACACCUUCGAGAGA | |
| 28 | | | | AAGGUAGA | |
| B_STMN2_ | A | CCTTCGAGAGAAAGGTAGAA | 89 | CCUGUUGUGAAUACUCUUUUAUAG | 152 |
| R_Splice2a_ | | | | GUAUCAAACAACCCUUCGAGAGAA | |
| 29 | | | | AGGUAGAA | |
| B_STMN2_ | G | AGAGAAAGGTAGAAAATAAG | 90 | CCUGUUGUGAAUACUCUUUUAUAG | 153 |
| R_Splice2a_ | | | | GUAUCAAACAACAGAGAAAGGUAG | |
| 30 | | | | AAAAUAAG | |
| B_STMN2_ | A | GAGCCAAATTCTTATTTTCT | 91 | CCUGUUGUGAAUACUCUUUUAUAG | 154 |
| R_Splice2a_ | | | | GUAUCAAACAACGAGCCAAAUUCU | |
| 41 | | | | UAUUUUCU | |
| B_STMN2_ | G | AGCCAAATTCTTATTTTCTA | 92 | CCUGUUGUGAAUACUCUUUUAUAG | 155 |
| R_Splice2a_ | | | | GUAUCAAACAACAGCCAAAUUCUU | |
| 42 | | | | AUUUUCUA | |
| B_STMN2_ | A | GCCAAATTCTTATTTTCTAC | 93 | CCUGUUGUGAAUACUCUUUUAUAG | 156 |
| R_Splice2a_ | | | | GUAUCAAACAACGCCAAAUUCUUA | |
| 43 | | | | UUUUCUAC | |
| B_STMN2_ | G | CCAAATTCTTATTTTCTACC | 94 | CCUGUUGUGAAUACUCUUUUAUAG | 157 |
| R_Splice2a_ | | | | GUAUCAAACAACCCAAAUUCUUAU | |
| 44 | | | | UUUCUACC | |
| B_STMN2_ | A | AATTCTTATTTTCTACCTTT | 95 | CCUGUUGUGAAUACUCUUUUAUAG | 158 |
| R_Splice2a_ | | | | GUAUCAAACAACAAUUCUUAUUUU | |
| 45 | | | | CUACCUUU | |
| B_STMN2_ | A | ATTCTTATTTTCTACCTTTC | 96 | CCUGUUGUGAAUACUCUUUUAUAG | 159 |
| R_Splice2a_ | | | | GUAUCAAACAACAUUCUUAUUUUC | |
| 46 | | | | UACCUUUC | |
| B_STMN2_ | A | TTCTTATTTTCTACCTTTCT | 97 | CCUGUUGUGAAUACUCUUUUAUAG | 160 |
| R_Splice2a_ | | | | GUAUCAAACAACUUCUUAUUUUCU | |
| 47 | | | | ACCUUUCU | |
| B_STMN2_ | A | TTTTCTACCTTTCTCTCGAA | 98 | CCUGUUGUGAAUACUCUUUUAUAG | 161 |
| R_Splice2a_ | | | | GUAUCAAACAACUUUUCUACCUUU | |
| 48 | | | | CUCUCGAA | |
| B_STMN2_ | A | CCTTTCTCTCGAAGGTCTTC | 99 | CCUGUUGUGAAUACUCUUUUAUAG | 162 |
| R_Splice2a_ | | | | GUAUCAAACAACCCUUUCUCUCGA | |
| 49 | | | | AGGUCUUC | |
| B_STMN2_ | G | AAGGTCTTCTGCCGAGTCCT | 100 | CCUGUUGUGAAUACUCUUUUAUAG | 163 |
| R_Splice2a_ | | | | GUAUCAAACAACAAGGUCUUCUGC | |
| 50 | | | | CGAGUCCU | |
| B_STMN2_ | A | AGGTCTTCTGCCGAGTCCTG | 101 | CCUGUUGUGAAUACUCUUUUAUAG | 164 |
| R_Splice2a_ | | | | GUAUCAAACAACAGGUCUUCUGCC | |
| 51 | | | | GAGUCCUG | |
| B_STMN2_ | A | GGTCTTCTGCCGAGTCCTGC | 102 | CCUGUUGUGAAUACUCUUUUAUAG | 165 |
| R_Splice2a_ | | | | GUAUCAAACAACGGUCUUCUGCCG | |
| 52 | | | | AGUCCUGC | |
| B_STMN2_ | G | GTCTTCTGCCGAGTCCTGCA | 103 | CCUGUUGUGAAUACUCUUUUAUAG | 166 |
| R_Splice2a_ | | | | GUAUCAAACAACGUCUUCUGCCGA | |
| 53 | | | | GUCCUGCA | |
| B_STMN2_ | G | TCTTCTGCCGAGTCCTGCAA | 104 | CCUGUUGUGAAUACUCUUUUAUAG | 167 |
| R_Splice2a_ | | | | GUAUCAAACAACUCUUCUGCCGAG | |
| 54 | | | | UCCUGCAA | |
| B_STMN2_ | G | CCGAGTCCTGCAATATGAAT | 105 | CCUGUUGUGAAUACUCUUUUAUAG | 168 |
| R_Splice2a_ | | | | GUAUCAAACAACCCGAGUCCUGCA | |
| 55 | | | | AUAUGAAU | |
| B_STMN2_ | G | AGTCCTGCAATATGAATATA | 106 | CCUGUUGUGAAUACUCUUUUAUAG | 169 |
| R_Splice2a_ | | | | GUAUCAAACAACAGUCCUGCAAUA | |
| 56 | | | | UGAAUAUA | |
| B_STMN2_ | A | GTCCTGCAATATGAATATAA | 107 | CCUGUUGUGAAUACUCUUUUAUAG | 170 |
| R_Splice2a_ | | | | GUAUCAAACAACGUCCUGCAAUAU | |
| 57 | | | | GAAUAUAA | |
| B_STMN2_ | G | TCCTGCAATATGAATATAAT | 108 | CCUGUUGUGAAUACUCUUUUAUAG | 171 |
| R_Splice2a_ | | | | GUAUCAAACAACUCCUGCAAUAUG | |
| 58 | | | | AAUAUAAU | |
| B_STMN2_ | A | TATGAATATAATTTTAAAAA | 109 | CCUGUUGUGAAUACUCUUUUAUAG | 172 |
| R_Splice2a_ | | | | GUAUCAAACAACUAUGAAUAUAAU | |
| 61 | | | | UUUAAAAA | |
| B_STMN2_ | A | ATATAATTTTAAAAATCCAA | 110 | CCUGUUGUGAAUACUCUUUUAUAG | 173 |
| R_Splice2a_ | | | | GUAUCAAACAACAUAUAAUUUUAA | |
| 64 | | | | AAAUCCAA | |
| B_STMN2_ | A | TATAATTTTAAAAATCCAAT | 111 | CCUGUUGUGAAUACUCUUUUAUAG | 174 |
| R_Splice2a_ | | | | GUAUCAAACAACUAUAAUUUUAAA | |
| 65 | | | | AAUCCAAU | |
| B_STMN2_ | A | TAATTTTAAAAATCCAATTA | 112 | CCUGUUGUGAAUACUCUUUUAUAG | 175 |
| R_Splice2a_ | | | | GUAUCAAACAACUAAUUUUAAAAA | |
| 66 | | | | UCCAAUUA | |
| B_STMN2_ | A | ATTTTAAAAATCCAATTAAG | 113 | CCUGUUGUGAAUACUCUUUUAUAG | 176 |
| R_Splice2a_ | | | | GUAUCAAACAACAUUUUAAAAAUC | |
| 67 | | | | CAAUUAAG | |
| B_STMN2_ | A | TTTTAAAAATCCAATTAAGA | 114 | CCUGUUGUGAAUACUCUUUUAUAG | 177 |
| R_Splice2a_ | | | | GUAUCAAACAACUUUUAAAAAUCC | |
| 68 | | | | AAUUAAGA | |
| B_STMN2_ | A | AAAATCCAATTAAGAGAGAG | 115 | CCUGUUGUGAAUACUCUUUUAUAG | 178 |
| R_Splice2a_ | | | | GUAUCAAACAACAAAAUCCAAUUA | |
| 69 | | | | AGAGAGAG | |
| B_STMN2_ | A | AAATCCAATTAAGAGAGAGT | 116 | CCUGUUGUGAAUACUCUUUUAUAG | 179 |
| R_Splice2a_ | | | | GUAUCAAACAACAAAUCCAAUUAA | |
| 70 | | | | GAGAGAGU | |
| B_STMN2_ | A | AATCCAATTAAGAGAGAGTG | 117 | CCUGUUGUGAAUACUCUUUUAUAG | 180 |
| R_Splice2a_ | | | | GUAUCAAACAACAAUCCAAUUAAG | |
| 71 | | | | AGAGAGUG | |
| B_STMN2_ | A | ATCCAATTAAGAGAGAGTGA | 118 | CCUGUUGUGAAUACUCUUUUAUAG | 181 |
| R_Splice2a_ | | | | GUAUCAAACAACAUCCAAUUAAGA | |
| 72 | | | | GAGAGUGA | |
| B_STMN2_ | A | TCCAATTAAGAGAGAGTGAT | 119 | CCUGUUGUGAAUACUCUUUUAUAG | 182 |
| R_Splice2a_ | | | | GUAUCAAACAACUCCAAUUAAGAG | |
| 73 | | | | AGAGUGAU | |

*The RNA guides for Nuclease B includes the nuclease-binding fragment of CCUGUUGUGAAUACUCUUUUAUAGGUAUCAAACAAC (SEQ ID NO: 183). The spacer sequence in each guide RNA is underlined.

TABLE 5
RNA Guide and Target Sequences for Nuclease C

| Reference | | | SEQ | | SEQ |
|---|---|---|---|---|---|
| Name | PAM | Target Sequence | ID NO | Guide Sequence* | ID NO |
| C_STMN2_Splice | TTC | TGCCCCATCACTCTCTCTTA | 184 | GGUCCCAUCGGAACGGGUUGU | 220 |
| 2a_1 | | | | GGUUCCGACUGCCCCAUCACU | |
| | | | | CUCUCUUA | |
| C_STMN2_Splice | CTG | CCCCATCACTCTCTCTTAAT | 185 | GGUCCCAUCGGAACGGGUUGU | 221 |
| 2a_2 | | | | GGUUCCGACCCCCAUCACUCU | |
| | | | | CUCUUAAU | |
| C_STMN2_Splice | CTC | TCTCTTAATTGGATTTTTAA | 186 | GGUCCCAUCGGAACGGGUUGU | 222 |
| 2a_4 | | | | GGUUCCGACUCUCUUAAUUGG | |
| | | | | AUUUUUAA | |
| C_STMN2_Splice | CTC | TCTTAATTGGATTTTTAAAA | 187 | GGUCCCAUCGGAACGGGUUGU | 223 |
| 2a_5 | | | | GGUUCCGACUCUUAAUUGGAU | |
| | | | | UUUUAAAA | |
| C_STMN2_Splice | TTA | ATTGGATTTTTAAAATTATA | 188 | GGUCCCAUCGGAACGGGUUGU | 224 |
| 2a_8 | | | | GGUUCCGACAUUGGAUUUUUA | |
| | | | | AAAUUAUA | |
| C_STMN2_Splice | TTT | TTAAAATTATATTCATATTG | 189 | GGUCCCAUCGGAACGGGUUGU | 225 |
| 2a_12 | | | | GGUUCCGACUUAAAAUUAUAU | |
| | | | | UCAUAUUG | |
| C_STMN2_Splice | TTT | TAAAATTATATTCATATTGC | 190 | GGUCCCAUCGGAACGGGUUGU | 226 |
| 2a_13 | | | | GGUUCCGACUAAAAUUAUAUU | |
| | | | | CAUAUUGC | |
| C_STMN2_Splice | TTT | AAAATTATATTCATATTGCA | 191 | GGUCCCAUCGGAACGGGUUGU | 227 |
| 2a_14 | | | | GGUUCCGACAAAAUUAUAUUC | |
| | | | | AUAUUGCA | |
| C_STMN2_Splice | TTA | AAATTATATTCATATTGCAG | 192 | GGUCCCAUCGGAACGGGUUGU | 228 |
| 2a_15 | | | | GGUUCCGACAAAUUAUAUUCA | |
| | | | | UAUUGCAG | |
| C_STMN2_Splice | TTA | TATTCATATTGCAGGACTCG | 193 | GGUCCCAUCGGAACGGGUUGU | 229 |
| 2a_17 | | | | GGUUCCGACUAUUCAUAUUGC | |
| | | | | AGGACUCG | |
| C_STMN2_Splice | TTC | ATATTGCAGGACTCGGCAGA | 194 | GGUCCCAUCGGAACGGGUUGU | 230 |
| 2a_20 | | | | GGUUCCGACAUAUUGCAGGAC | |
| | | | | UCGGCAGA | |
| C_STMN2_Splice | TTG | CAGGACTCGGCAGAAGACCT | 195 | GGUCCCAUCGGAACGGGUUGU | 231 |
| 2a_23 | | | | GGUUCCGACCAGGACUCGGCA | |
| | | | | GAAGACCU | |
| C_STMN2_Splice | CTC | GGCAGAAGACCTTCGAGAGA | 196 | GGUCCCAUCGGAACGGGUUGU | 232 |
| 2a_24 | | | | GGUUCCGACGGCAGAAGACCU | |
| | | | | UCGAGAGA | |
| C_STMN2_Splice | CTT | CGAGAGAAAGGTAGAAAATA | 197 | GGUCCCAUCGGAACGGGUUGU | 233 |
| 2a_25 | | | | GGUUCCGACCGAGAGAAAGGU | |
| | | | | AGAAAAUA | |
| C_STMN2_Splice | TTC | GAGAGAAAGGTAGAAAATAA | 198 | GGUCCCAUCGGAACGGGUUGU | 234 |
| 2a_26 | | | | GGUUCCGACGAGAGAAAGGUA | |
| | | | | GAAAAUAA | |
| C_STMN2_Splice | TTC | TTATTTTCTACCTTTCTCTC | 199 | GGUCCCAUCGGAACGGGUUGU | 235 |
| 2a_28 | | | | GGUUCCGACUUAUUUUCUACC | |
| | | | | UUUCUCUC | |
| C_STMN2_Splice | CTT | ATTTTCTACCTTTCTCTCGA | 200 | GGUCCCAUCGGAACGGGUUGU | 236 |
| 2a_29 | | | | GGUUCCGACAUUUUCUACCUU | |
| | | | | UCUCUCGA | |
| C_STMN2_Splice | TTA | TTTTCTACCTTTCTCTCGAA | 201 | GGUCCCAUCGGAACGGGUUGU | 237 |
| 2a_30 | | | | GGUUCCGACUUUUCUACCUUU | |
| | | | | CUCUCGAA | |
| C_STMN2_Splice | TTT | TCTACCTTTCTCTCGAAGGT | 202 | GGUCCCAUCGGAACGGGUUGU | 238 |
| 2a_32 | | | | GGUUCCGACUCUACCUUUCUC | |
| | | | | UCGAAGGU | |
| C_STMN2_Splice | TTT | CTACCTTTCTCTCGAAGGTC | 203 | GGUCCCAUCGGAACGGGUUGU | 239 |
| 2a_33 | | | | GGUUCCGACCUACCUUUCUCU | |
| | | | | CGAAGGUC | |
| C_STMN2_Splice | TTC | TACCTTTCTCTCGAAGGTCT | 204 | GGUCCCAUCGGAACGGGUUGU | 240 |
| 2a_34 | | | | GGUUCCGACUACCUUUCUCUC | |
| | | | | GAAGGUCU | |
| C_STMN2_Splice | CTA | CCTTTCTCTCGAAGGTCTTC | 205 | GGUCCCAUCGGAACGGGUUGU | 241 |
| 2a_35 | | | | GGUUCCGACCCUUUCUCUCGA | |
| | | | | AGGUCUUC | |
| C_STMN2_Splice | CTT | TCTCTCGAAGGTCTTCTGCC | 206 | GGUCCCAUCGGAACGGGUUGU | 242 |
| 2a_36 | | | | GGUUCCGACUCUCUCGAAGGU | |
| | | | | CUUCUGCC | |
| C_STMN2_Splice | TTT | CTCTCGAAGGTCTTCTGCCG | 207 | GGUCCCAUCGGAACGGGUUGU | 243 |
| 2a_37 | | | | GGUUCCGACCUCUCGAAGGUC | |
| | | | | UUCUGCCG | |
| C_STMN2_Splice | TTC | TCTCGAAGGTCTTCTGCCGA | 208 | GGUCCCAUCGGAACGGGUUGU | 244 |
| 2a_38 | | | | GGUUCCGACUCUCGAAGGUCU | |
| | | | | UCUGCCGA | |
| C_STMN2_Splice | CTC | TCGAAGGTCTTCTGCCGAGT | 209 | GGUCCCAUCGGAACGGGUUGU | 245 |
| 2a_39 | | | | GGUUCCGACUCGAAGGUCUUC | |
| | | | | UGCCGAGU | |
| C_STMN2_Splice | CTC | GAAGGTCTTCTGCCGAGTCC | 210 | GGUCCCAUCGGAACGGGUUGU | 246 |
| 2a_40 | | | | GGUUCCGACGAAGGUCUUCUG | |
| | | | | CCGAGUCC | |
| C_STMN2_Splice | CTT | CTGCCGAGTCCTGCAATATG | 211 | GGUCCCAUCGGAACGGGUUGU | 247 |
| 2a_41 | | | | GGUUCCGACCUGCCGAGUCCU | |
| | | | | GCAAUAUG | |
| C_STMN2_Splice | TTC | TGCCGAGTCCTGCAATATGA | 212 | GGUCCCAUCGGAACGGGUUGU | 248 |
| 2a_42 | | | | GGUUCCGACUGCCGAGUCCUG | |
| | | | | CAAUAUGA | |
| C_STMN2_Splice | CTG | CCGAGTCCTGCAATATGAAT | 213 | GGUCCCAUCGGAACGGGUUGU | 249 |
| 2a_43 | | | | GGUUCCGACCCGAGUCCUGCA | |
| | | | | AUAUGAAU | |
| C_STMN2_Splice | CTG | CAATATGAATATAATTTTAA | 214 | GGUCCCAUCGGAACGGGUUGU | 250 |
| 2a_44 | | | | GGUUCCGACCAAUAUGAAUAU | |
| | | | | AAUUUUAA | |
| C_STMN2_Splice | ATG | AATATAATTTTAAAAATCCA | 215 | GGUCCCAUCGGAACGGGUUGU | 251 |
| 2a_46 | | | | GGUUCCGACAAUAUAAUUUUA | |
| | | | | AAAAUCCA | |
| C_STMN2_Splice | ATA | ATTTTAAAAATCCAATTAAG | 216 | GGUCCCAUCGGAACGGGUUGU | 252 |
| 2a_48 | | | | GGUUCCGACAUUUUAAAAAUC | |
| | | | | CAAUUAAG | |
| C_STMN2_Splice | TTT | TAAAAATCCAATTAAGAGAG | 217 | GGUCCCAUCGGAACGGGUUGU | 253 |
| 2a_50 | | | | GGUUCCGACUAAAAAUCCAAU | |
| | | | | UAAGAGAG | |
| C_STMN2_Splice | TTT | AAAAATCCAATTAAGAGAGA | 218 | GGUCCCAUCGGAACGGGUUGU | 254 |
| 2a_51 | | | | GGUUCCGACAAAAAUCCAAUU | |
| | | | | AAGAGAGA | |
| C_STMN2_Splice | TTA | AAAATCCAATTAAGAGAGAG | 219 | GGUCCCAUCGGAACGGGUUGU | 37 |
| 2a_52 | | | | GGUUCCGACAAAAUCCAAUUA | |
| | | | | AGAGAGAG | |

*The RNA guides for Nuclease C includes the nuclease-binding fragment of GGUCCCAUCGGAACGGGUUGUGGUUCCGAC (SEQ ID NO: 38). The spacer sequence in each guide RNA is underlined.

### Editing Efficiency

The exemplary Type V nucleases A, B, and C listed in Table 1 were individually cloned into a plasmid comprising a CMV promoter. Fragments coding for RNA guides of Tables 3-5 were cloned into a pUC19 backbone (New England Biolabs) that contained a hU6 promoter. The plasmids were then maxi-prepped and diluted.

Approximately 16 hours prior to transfection, 25,000 HEK293T cells in DMEM/10% FBS+Pen/Strep (D10 media) were plated into each well of a 96-well plate. On the day of transfection, the cells were 70-90% confluent. For each well to be transfected, a mixture of LIPOFECTAMINE® 2000 transfection reagent (ThermoFisher) and Opti-MEM® reduced serum medium (ThermoFisher) was prepared and incubated at room temperature for 5 minutes (Solution 1). After incubation, the LIPOFECTAMINE® 2000: Opti-MEM® (transfection reagent (ThermoFisher): reduced serum medium (ThermoFisher)) mixture was added to a separate mixture containing nuclease plasmid, RNA guide plasmid, and Opti-MEM® reduced serum medium (ThermoFisher) (Solution 2). In the case of negative controls, the RNA guide plasmid was not included in Solution 2. Solution 1 and 2 were pipette mixed 8 times, then incubated at room temperature for 25 minutes. Following incubation, the Solution 1 and 2 mixture was added dropwise to each well of a 96-well plate containing the cells. 72 hours post transfection, cells were trypsinized by adding TRYPLE™ (recombinant cell-dissociation enzymes; ThermoFisher) to the center of each well and incubating at 37° C. for approximately 5 minutes. D10 media was then added to each well and mixed to resuspend cells. The resuspended cells were centrifuged at 500×g for 10 minutes to obtain a pellet, and the supernatant was discarded. QUICKEXTRACT™ (DNA extraction solution; Lucigen) extraction reagent was added to each well to lyse pelleted cells. Cells were incubated at 65° C. for 15 minutes, 68° C. for 15 minutes, and 98° C. for 10 minutes.

Samples for NGS were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were done with a 300 Cycle NEXTSEQ™ (Illumina) 500/550 High Output v2.5 Kit.

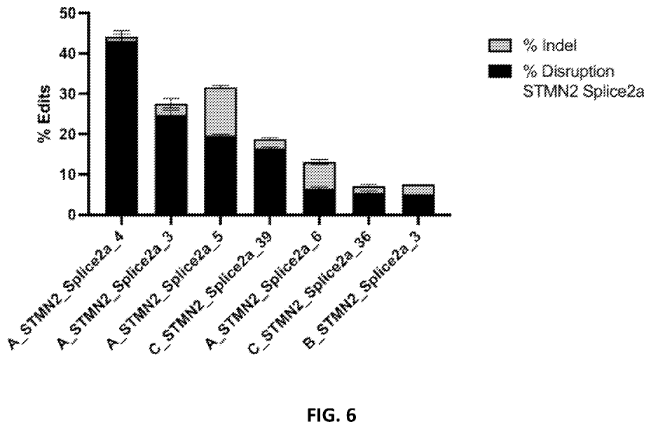

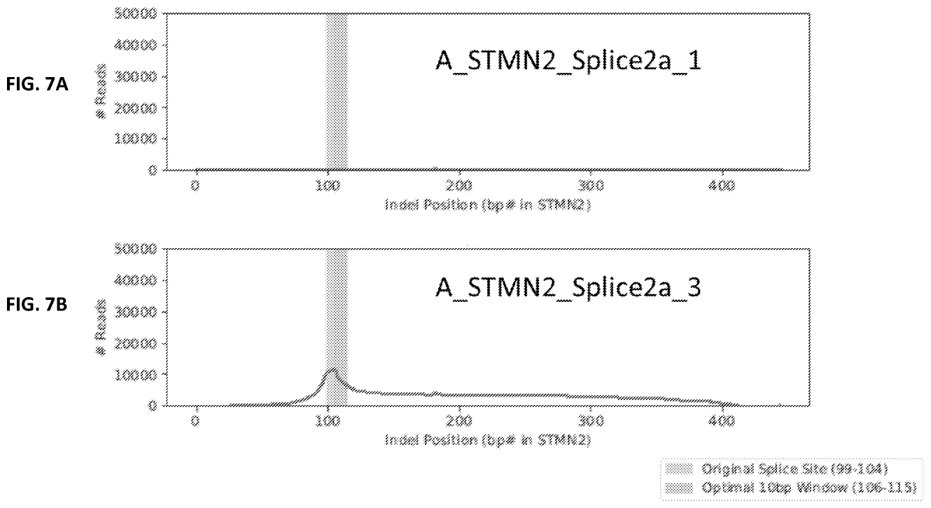

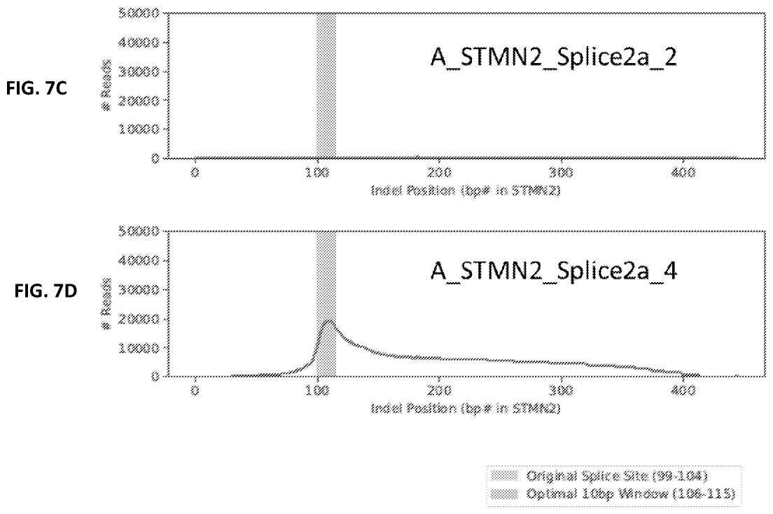

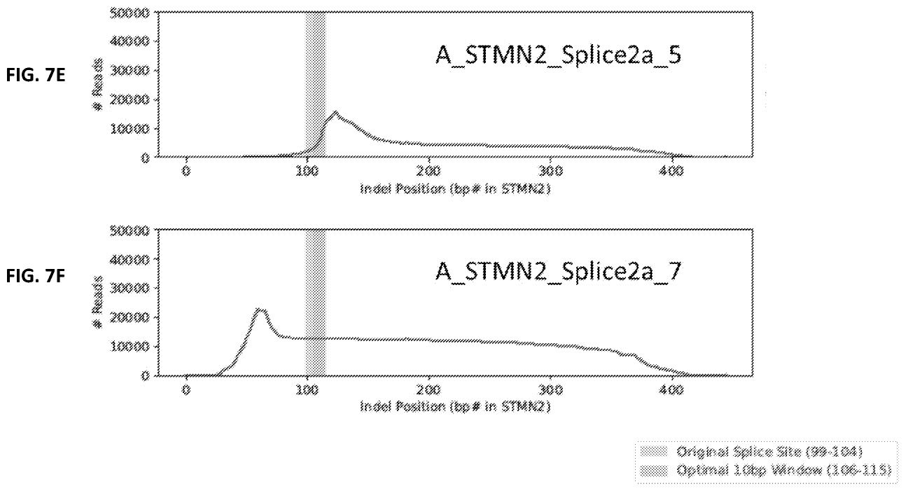

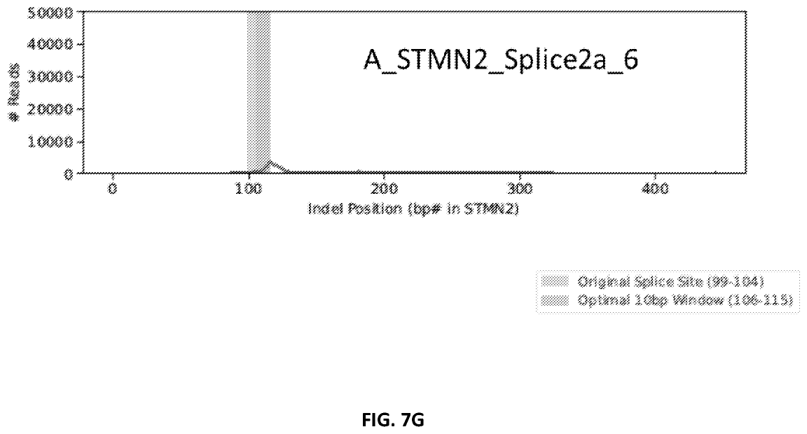

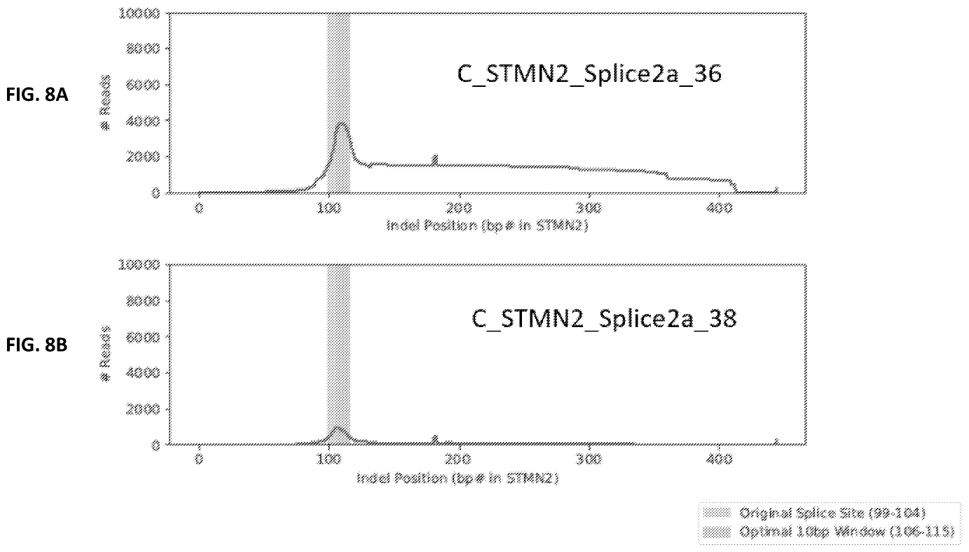

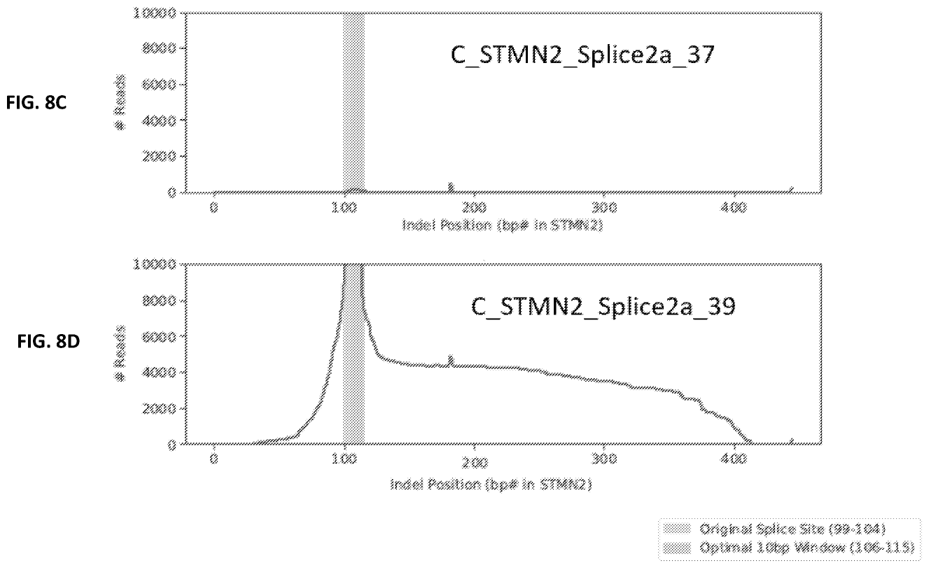

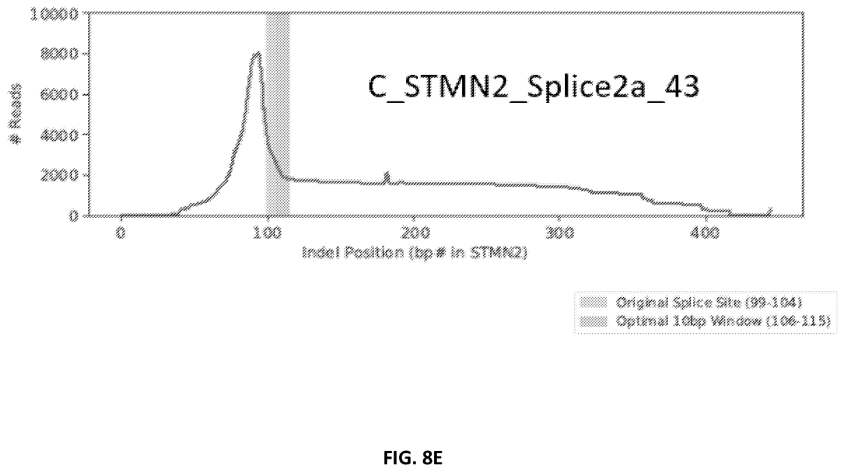

The percentage of NGS reads containing indels (% Indels) and the percentage of NGS reads containing indels within the 10-bp optimal window described herein (% Motif Disruption) for the RNA guides of Tables 2-4 are shown in Table 6. The seven RNA guides producing >5% Motif Disruption are shown in bold (Table 6) and further depicted in FIG. 6. Two additional RNA guides produced apparent % Motif Disruption >5%, however further evaluation revealed these elevated percentages (italicized) were due to high background (e.g., A_STMN2_Splice2a_7 (FIG. 7F), and C_STMN2_Splice2a_43 (FIG. 8E)).

TABLE 6
Disruption of STMN2 Splice2a by Nucleases A, B, and C.

| | | % Motif |
|---|---|---|
| Guide Reference Name | % Indels | Disruption |
| A_STMN2_Splice2a_1 | 1.52% | 0.02% |
| A_STMN2_Splice2a_2 | 1.37% | 0.01% |
| A_{—}STMN2_{—}Splice2a_{—}3 | 31.03% | 28.65% |
| A_{—}STMN2_{—}Splice2a_{—}4 | 47.65% | 46.93% |
| A_{—}STMN2_{—}Splice2a_{—}5 | 36.80% | 24.21% |
| A_{—}STMN2_{—}Splice2a_{—}6 | 11.17% | 5.84% |
| A_STMN2_Splice2a_7 | 50.61% | 23.53% |
| B_STMN2_Splice2a_1 | 2.33% | 0.02% |
| B_{—}STMN2_{—}Splice2a_{—}3 | 7.54% | 5.04% |
| B_STMN2_Splice2a_4 | 3.74% | 1.36% |
| B_STMN2_Splice2a_5 | 13.16% | 2.53% |
| B_STMN2_Splice2a_7 | 13.00% | 0.14% |
| B_STMN2_R_Splice2a_1 | 11.08% | 0.80% |
| B_STMN2_R_Splice2a_2 | 2.41% | 0.03% |
| B_STMN2_R_Splice2a_3 | 5.12% | 0.13% |
| B_STMN2_R_Splice2a_4 | 2.48% | 0.03% |
| B_STMN2_R_Splice2a_5 | 2.44% | 0.03% |
| B_STMN2_R_Splice2a_8 | 2.49% | 0.03% |
| B_STMN2_R_Splice2a_9 | 7.22% | 4.75% |
| B_STMN2_R_Splice2a_10 | 2.54% | 0.15% |
| B_STMN2_R_Splice2a_11 | 2.51% | 0.03% |
| B_STMN2_R_Splice2a_12 | 2.37% | 0.03% |
| B_STMN2_R_Splice2a_13 | 5.31% | 3.04% |
| B_STMN2_R_Splice2a_14 | 2.86% | 0.41% |
| B_STMN2_R_Splice2a_15 | 5.05% | 1.46% |
| B_STMN2_R_Splice2a_16 | 7.29% | 1.51% |
| B_STMN2_R_Splice2a_17 | 16.06% | 3.08% |
| B_STMN2_R_Splice2a_18 | 7.46% | 0.23% |
| B_STMN2_R_Splice2a_19 | 2.49% | 0.01% |
| B_STMN2_R_Splice2a_20 | 2.44% | 0.02% |
| B_STMN2_R_Splice2a_21 | 2.39% | 0.02% |
| B_STMN2_R_Splice2a_22 | 12.01% | 1.28% |
| B_STMN2_R_Splice2a_23 | 3.04% | 0.04% |
| B_STMN2_R_Splice2a_24 | 2.32% | 0.03% |
| B_STMN2_R_Splice2a_25 | 2.40% | 0.03% |
| B_STMN2_R_Splice2a_26 | 2.49% | 0.02% |
| B_STMN2_R_Splice2a_27 | 2.43% | 0.02% |
| B_STMN2_R_Splice2a_28 | 2.40% | 0.03% |
| B_STMN2_R_Splice2a_29 | 2.41% | 0.03% |
| B_STMN2_R_Splice2a_30 | 39.96% | 1.52% |
| B_STMN2_R_Splice2a_41 | 2.43% | 0.03% |
| B_STMN2_R_Splice2a_42 | 2.48% | 0.03% |
| B_STMN2_R_Splice2a_43 | 2.47% | 0.02% |
| B_STMN2_R_Splice2a_44 | 2.45% | 0.02% |
| B_STMN2_R_Splice2a_45 | 2.43% | 0.02% |
| B_STMN2_R_Splice2a_46 | 2.49% | 0.03% |
| B_STMN2_R_Splice2a_47 | 2.25% | 0.02% |
| B_STMN2_R_Splice2a_48 | 2.64% | 0.12% |
| B_STMN2_R_Splice2a_49 | 5.33% | 1.95% |
| B_STMN2_R_Splice2a_50 | 2.39% | 0.04% |
| B_STMN2_R_Splice2a_51 | 2.38% | 0.03% |
| B_STMN2_R_Splice2a_52 | 2.52% | 0.03% |
| B_STMN2_R_Splice2a_53 | 4.35% | 0.05% |
| B_STMN2_R_Splice2a_54 | 2.42% | 0.04% |
| B_STMN2_R_Splice2a_55 | 13.77% | 0.33% |
| B_STMN2_R_Splice2a_56 | 3.30% | 0.04% |
| B_STMN2_R_Splice2a_57 | 2.79% | 0.04% |
| B_STMN2_R_Splice2a_58 | 2.52% | 0.02% |
| B_STMN2_R_Splice2a_61 | 2.36% | 0.02% |
| B_STMN2_R_Splice2a_64 | 2.39% | 0.03% |
| B_STMN2_R_Splice2a_66 | 2.38% | 0.02% |
| B_STMN2_R_Splice2a_67 | 2.36% | 0.03% |
| B_STMN2_R_Splice2a_68 | 2.47% | 0.02% |
| B_STMN2_R_Splice2a_69 | 16.81% | 0.16% |
| B_STMN2_R_Splice2a_70 | 2.54% | 0.02% |
| B_STMN2_R_Splice2a_71 | 2.43% | 0.03% |
| B_STMN2_R_Splice2a_72 | 2.42% | 0.02% |
| B_STMN2_R_Splice2a_73 | 2.44% | 0.03% |
| C_STMN2_Splice2a_1 | 1.35% | 0.01% |
| C_STMN2_Splice2a_2 | 3.36% | 0.12% |
| C_STMN2_Splice2a_4 | 1.62% | 0.01% |
| C_STMN2_Splice2a_5 | 1.49% | 0.01% |
| C_STMN2_Splice2a_8 | 1.76% | 0.01% |
| C_STMN2_Splice2a_12 | 1.42% | 0.02% |
| C_STMN2_Splice2a_13 | 1.54% | 0.09% |
| C_STMN2_Splice2a_14 | 1.44% | 0.03% |
| C_STMN2_Splice2a_15 | 1.37% | 0.06% |
| C_STMN2_Splice2a_17 | 2.05% | 0.67% |
| C_STMN2_Splice2a_20 | 2.10% | 0.77% |
| C_STMN2_Splice2a_23 | 2.10% | 0.47% |
| C_STMN2_Splice2a_24 | 5.54% | 0.51% |
| C_STMN2_Splice2a_25 | 3.80% | 0.13% |
| C_STMN2_Splice2a_26 | 2.21% | 0.03% |
| C_STMN2_Splice2a_28 | 1.32% | 0.01% |
| C_STMN2_Splice2a_29 | 1.51% | 0.06% |
| C_STMN2_Splice2a_30 | 1.62% | 0.07% |
| C_STMN2_Splice2a_32 | 3.34% | 1.23% |
| C_STMN2_Splice2a_33 | 2.28% | 0.75% |
| C_STMN2_Splice2a_34 | 2.71% | 1.26% |
| C_STMN2_Splice2a_35 | 2.75% | 1.39% |
| C_{—}STMN2_{—}Splice2a_{—}36 | 9.51% | 8.23% |
| C_STMN2_Splice2a_37 | 1.80% | 0.45% |
| C_STMN2_Splice2a_38 | 3.57% | 2.19% |
| C_{—}STMN2_{—}Splice2a_{—}39 | 26.18% | 24.55% |
| C_STMN2_Splice2a_40 | 4.23% | 2.40% |
| C_STMN2_Splice2a_41 | 1.35% | 0.01% |
| C_STMN2_Splice2a_42 | 7.01% | 1.10% |
| C_STMN2_Splice2a_43 | 17.72% | 6.05% |
| C_STMN2_Splice2a_44 | 4.45% | 0.64% |
| C_STMN2_Splice2a_46 | 1.36% | 0.00% |
| C_STMN2_Splice2a_48 | 1.68% | 0.03% |
| C_STMN2_Splice2a_50 | 2.08% | 0.09% |
| C_STMN2_Splice2a_51 | 1.98% | 0.02% |
| C_STMN2_Splice2a_52 | 2.18% | 0.02% |

When co-delivered with Nuclease A, RNA guides A_STMN2_Splice2a_3, A_STMN2_Splice2a_4, A_STMN2_Splice2a_5, and A_STMN2_Splice2a_6 resulted in greater than 5% motif disruption.

RNA guides used in this Example that induce indels within the 3′ splice site disclosed herein and/or the optimal disruption window identified in Example 3 above allow for functional STMN2 recovery (e.g., recovery of full-length STMN2 transcripts) in cells such as SH-SY5Y cells and neurons. The Nuclease A guides were selected with the highest overall indel rates and high % motif disruption to take forward for testing in SH-SY5Y cells (Example 5).

### Computational Analysis of Indels Induced by Type V Nucleases

A computational analysis was performed on samples described in Example 4 above to determine how indels within or near the 3′ splice site of exon 2a correlate with full-length STMN2 recovery. The full-length STMN2 transcript refers to the mRNA containing exons 1-5, which encodes a functional STMN2 protein. The methodology used here is similar to that described in Example 3.

FIGS. 7A-7G show the positions of indels induced by a Nuclease A and seven different STMN2 guides within the STMN2 amplicon (x-axis). The number of NGS reads is on the y-axis. The grey bar on the left indicates the position of the literature-defined splice site (99-104). The grey bar on the right indicates the position of the optimal 10-nt disruption window identified above (106-115). Consistent with the indel data in Table 4 guide A_STMN2_Splice2a_4 (FIG. 7D, lower panel) showed the greatest % Motif disruption with the peak reads in the optimal 10-nt window. Overall, the highest % motif disruption corresponded to increase number of reads in the optimal 10-nt disruption window.

FIGS. 8A-8E show the positions of indels induced by a Nuclease C and four representative STMN guides within the STMN2 amplicon (x-axis). The number of NGS reads is on the y-axis. The grey bar on the left indicates the position of the literature-defined splice site. The grey bar on the right indicates the position of the optimal 10-nt disruption window identified above. The plots show that the greatest % Motif disruption correlates with the peak reads in the optimal 10-nt window.

FIGS. 7F and 8E illustrate guides with high background and Indels that peak outside of either the canonical splice site or optimal window defined herein, therefore these gRNAs were not taken forward for additional testing.

The Indel patterns observed in FIGS. 7A-7G and FIGS. 8A-8E in conjunction with the % motif disruption seen in Table 6 support the selection of Nuclease A guides for further studies of functional STMN2 recovery in SH-SY5Y cells and neurons.

### Example 5—Targeting STMN2 Exon 2A Splice Site by a Type V CRISPR Nuclease in SH-SY5Y Cells

This Example shows that using a Type V CRISPR nuclease (Nuclease D listed in Table 1 above) complexed with an RNA guide (Table 3) designed to disrupt the exon 2A splice site of STMN2 (the 3′ splice site in intron 1 as disclosed herein) results in a reduction of the STMN2 exon 2A splice variant, which corresponds to an increase in full-length STMN2 transcripts.

SH-SY5Y cells were cultured for 48 hours to a confluency of 70-80% in DMEM/F12 (Gibco #10565018) containing 10% FBS (Hyclone, Heat inactivated #SH30071.03). Cells were lifted with TrypLE (Gibco), counted, rinsed with PBS, and resuspended in Lonza SF nucleofection buffer+supplement (Lonza, V4XC-2024). Two million cells were used per electroporation reaction.

Each RNA guide of Table 3 was designed to target a 3′ splice site of STMN2 intron 1 (the splice site for exon 2A) and was complexed with Nuclease D. A non-targeting control guide was also used (CUUGUUGUAUAUGUCCUUUUAUAGGUAUUAAACAACAGUGCGUA CGAGCUCGGACG; SEQ ID NO: 260). RNP complexes were generated by mixing the Type V CRISPR nuclease (in 20 mM HEPES pH 7.8, 500 mM NaCl, 10% Glycerol, 0.5 mM TCEP) with an RNA guide (in 250 mM NaCl) on ice at a 1:2.5 molar ratio for 60 minutes. RNPs were added to each reaction at a final concentration of 10 μM nuclease and 25 μM RNA guide, in the presence of 1 μM siTARDBP (siTDP-43 RNA; Horizon Discovery Biosciences ON-TARGETplus Human TARDP [GCUCAAGCAUGGAUUCUAA (SEQ ID NO:7), CAAUCAAGGUAGUAAUAUG (SEQ ID NO:8), GGGCUUCGCUACAGGAAUC (SEQ ID NO: 9); and CAGGGUGGAUUUGGUAAUA (SEQ ID NO:10)]) or siNon-targeting Pool (siCont RNA; Horizon Discovery Biosciences ON-TARGETplus Non-targeting Pool).

The cuvettes were electroporated using an electroporation device (program CA-137, Lonza 4D-nucleofector). Following electroporation, cells were allowed to rest for 10 minutes before being added to pre-warmed culture medium and mixed gently by pipetting. The cells were then incubated at 37° C. for 72 hours. Next, cells were rinsed with PBS and lysed with RLT+ (Qiagen). RNA isolation was performed using QIAshredder columns and RNeasy plus Micro Kit (Qiagen). RNA (2 μg) was converted to cDNA using High-Capacity cDNA Reverse Transcription kit (Applied Biosystems). Quantitative reverse transcription PCR was performed using Faststart Universal Probe Master (Rox), (Roche), and TaqMan probes for TARDP (Hs00606522_m1, Thermo Fisher), STMN2 (Hs00975900_m1, Thermo Fisher) and GAPDH (Hs02786624_g1, Thermo Fisher). The STMN2 exon 2A splice variant was detected using a custom TaqMan probe and PCR primers (Baughn et al., Science 379 (2023): Forward Primer CTTTCTCTAGCACGGTCCCAC (SEQ ID NO: 257), Reverse Primer ATGCTCACACAGAGAGCCAAATT (SEQ ID NO: 258), Probe CTCTCGAAGGTCTTCTGCCG (SEQ ID NO: 259).

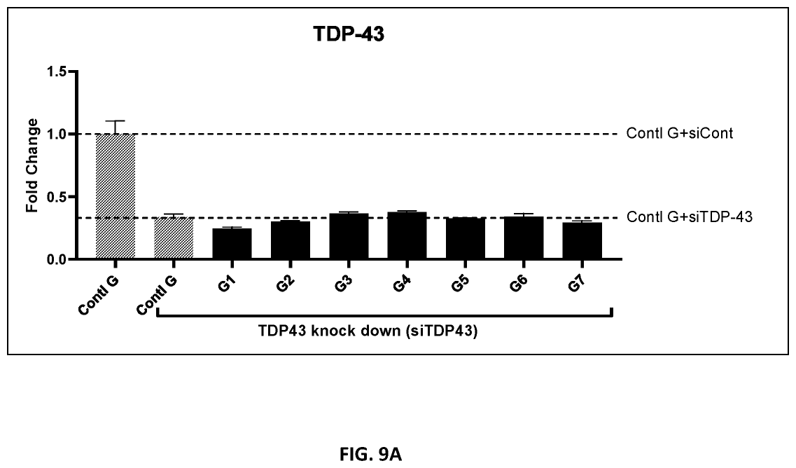

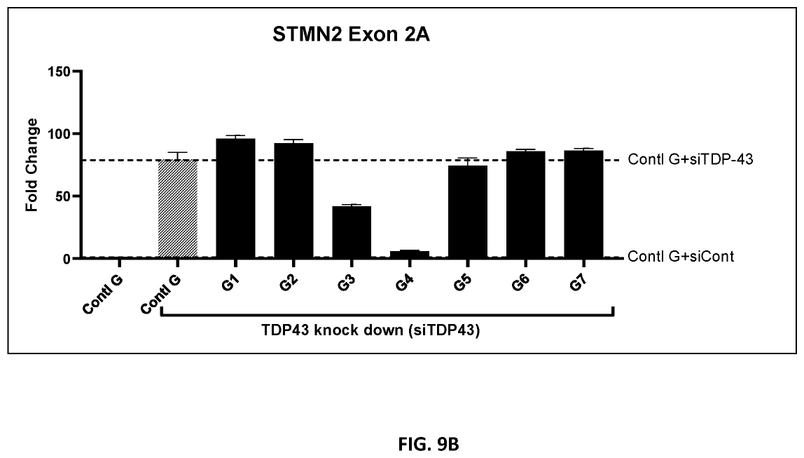

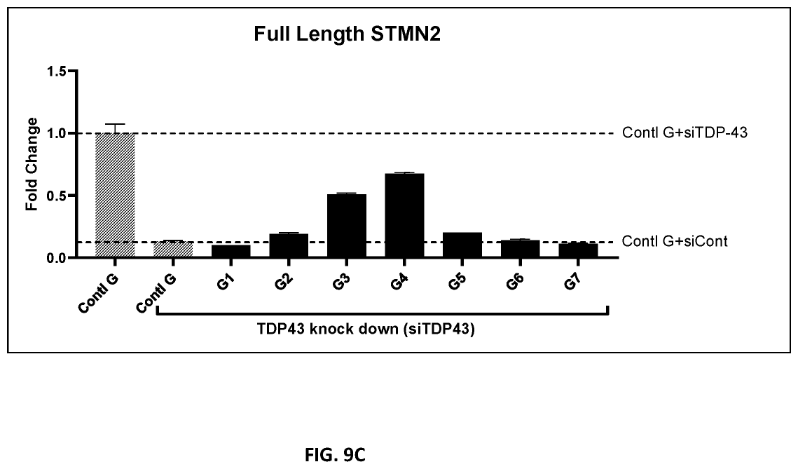

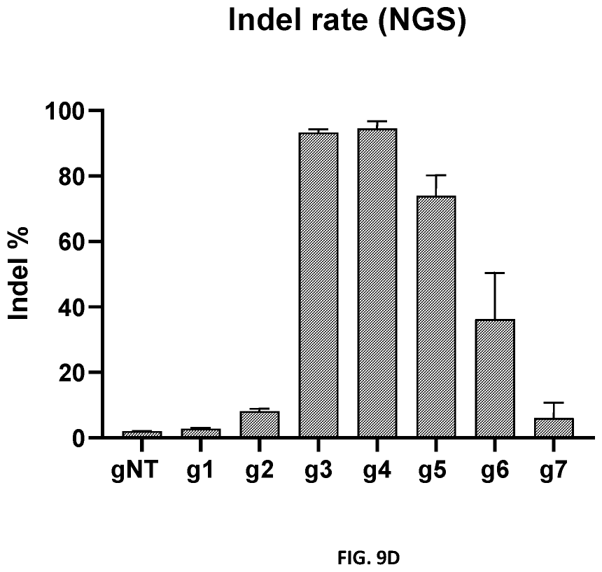

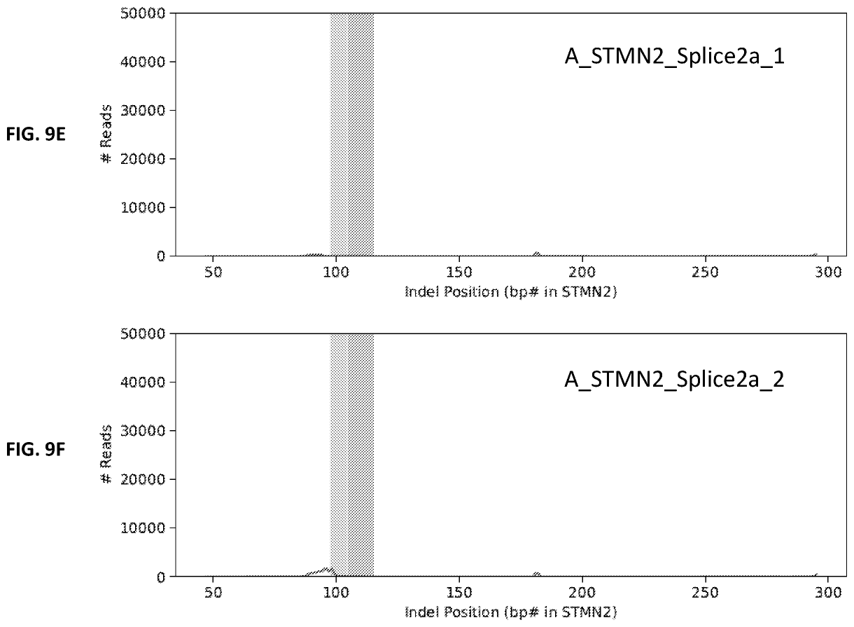

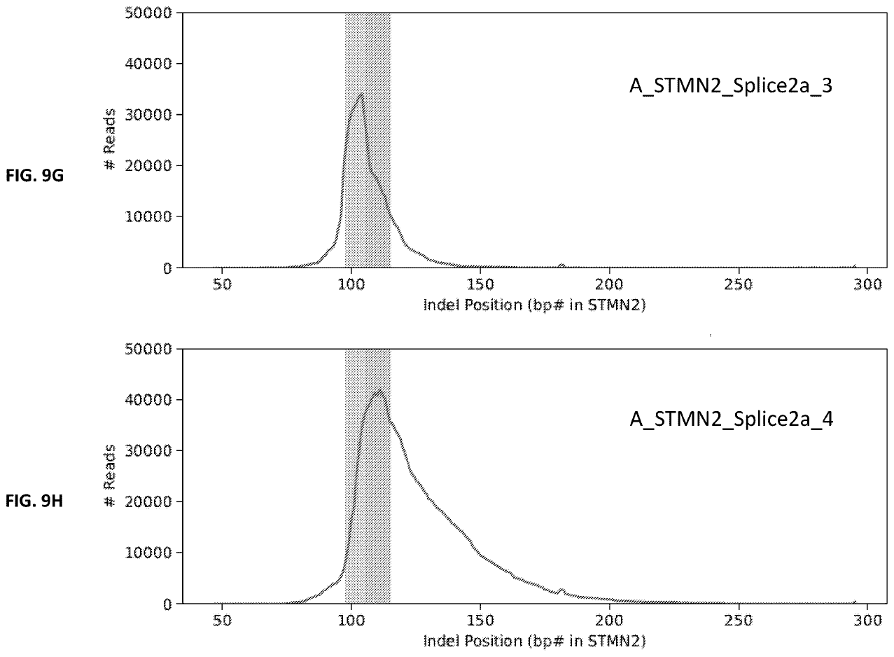

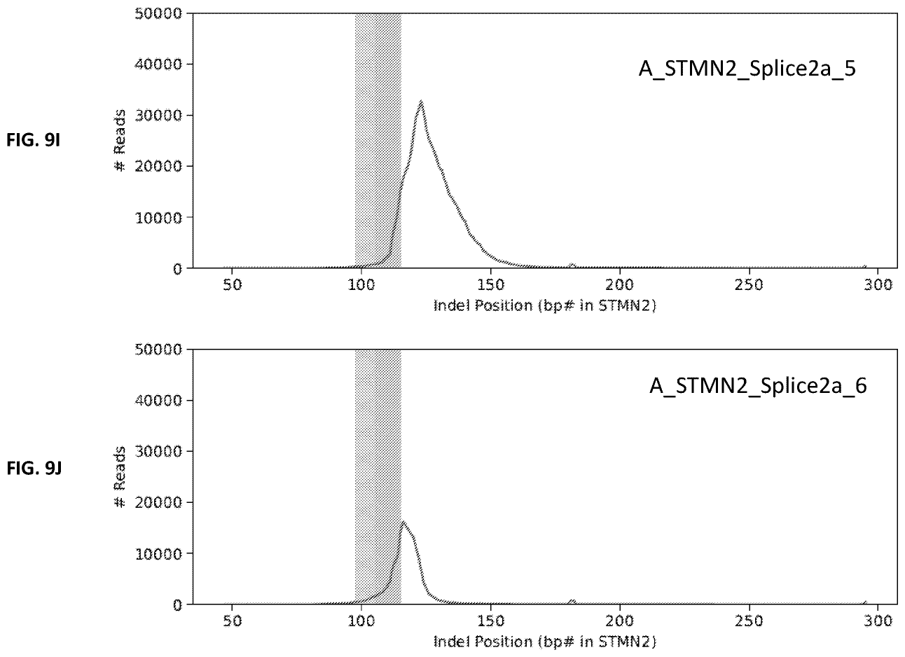

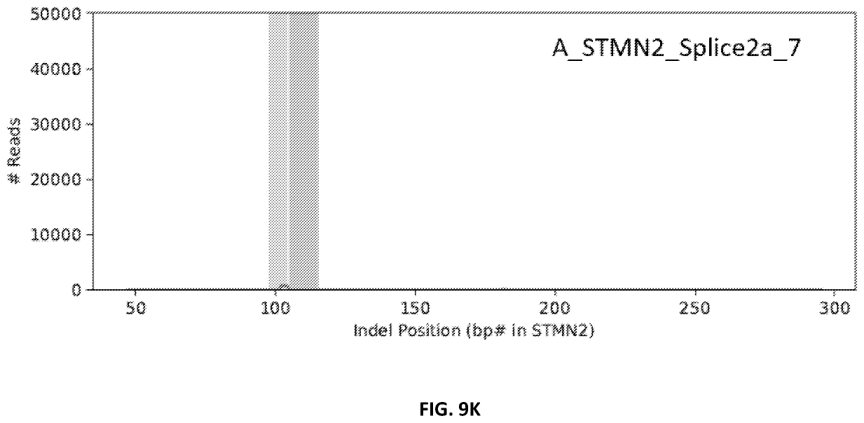

The RNA guides and the corresponding target sequences are provided in Table 3 above, for reference of the sample identifiers, see, FIGS. 9A-9K and Table 7 below.

TABLE 7
Sample Identifiers and Corresponding RNA Guides

| Sample | Guide reference name |
|---|---|
| Control G | Non-targeting control guide |
| G1 | A_STMN2_Splice2a_1 |
| G2 | A_STMN2_Splice2a_2 |
| G3 | A_STMN2_Splice2a_3 |
| G4 | A_STMN2_Splice2a_4 |
| G5 | A_STMN2_Splice2a_5 |
| G6 | A_STMN2_Splice2a_6 |
| G7 | A_STMN2_Splice2a_7 |

As shown in FIG. 9A, a 3-fold TDP-43 knockdown was observed in SH-SY5Y cells using siTDP-43 RNA. Knockdown of TDP-43 in the presence of a non-targeting control RNP complex resulted in an 80-fold increase in the STMN2 exon 2A splice variant and in over a 7.5-fold decrease in the STMN2 full length transcript (FIG. 9B and FIG. 9C). The exon 2A increase was significantly mitigated by RNP guides A_STMN2_Splice2a_3 and A_STMN2_Splice2a_4 (FIG. 9B). Significant restoration of the decreased full length STMN2 signal, due to TDP-43 depletion, was observed with RNA guides A_STMN2_Splice2a_3 and A_STMN2_Splice2a_4 (FIG. 9C). Indel activities of Nuclease A with various gRNAs as indicated in SH-SYSY cells are shown in FIG. 9D. The computational analysis of indel positions induced in the STMN2 sequence by a particular guide in association with Nuclease A in SH-SY5Y cells are shown in FIGS. 9E-9K. The data demonstrate that the guides with the greatest increase in full length STMN2 transcript (FIG. 9C) and corresponding decrease in the exon 2A transcript (FIG. 9B) also exhibited the highest indel activity in the desired optimal disruption window (FIGS. 9G and 9H).

### Example 6—STMN2 Exon 2A Splice Site Disruption by CRISPR Nuclease Variants in SH-SY5Y Cells

This Example compares disruption of the exon 2A splice site of STMN2 and the effect on expression levels of the STMN2 exon 2A splice variant and full length STMN2 with the Type V CRISPR nuclease variants of Nuclease A and Nuclease D listed in Table 1 above.

SH-SY5Y cells were cultured for 48 hours to a confluency of 70-80% in DMEM/F12 (Gibco #10565018) containing 10% FBS (Hyclone, Heat inactivated #SH30071.03). Cells were lifted with TrypLE (Gibco), counted, rinsed with PBS, and resuspended in Lonza SF nucleofection buffer+supplement (Lonza, V4XC-2024) at a concentration of 20,000 cells/uL.

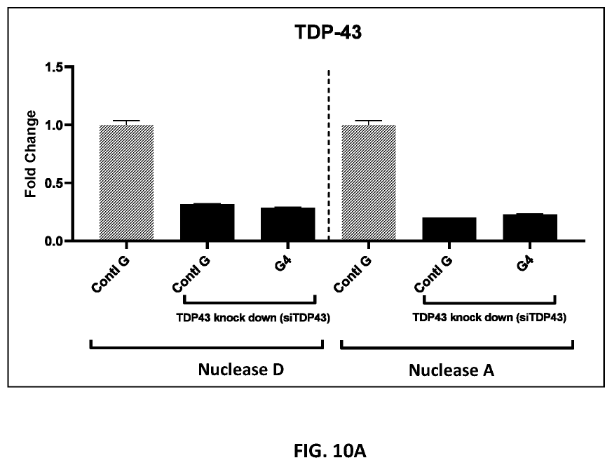

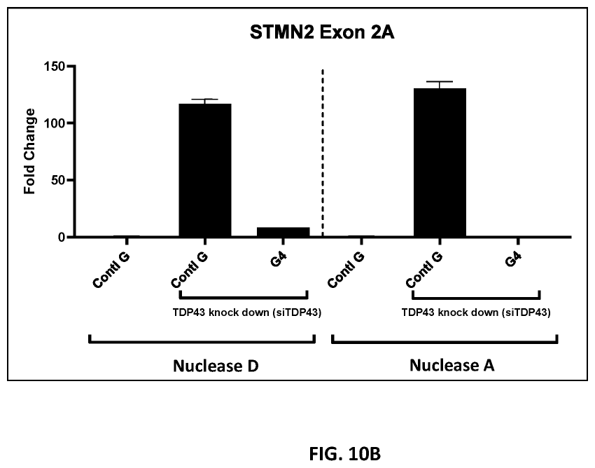

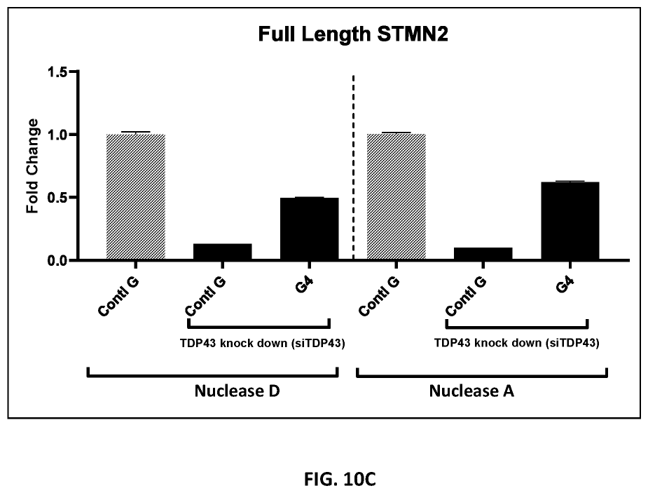

Two million cells were co-nucleofected with RNP complexes containing RNA guide A_STMN2_Splice2a_4 (see Table 3 above, G4 in FIGS. 10A-10C) or a non-targeting control guide

(CUUGUUGUAUAUGUCCUUUUAUAGGUAUUAAACAACAGUGCG
UACGAGCUCGGACG; SEQ ID NO: 260: Contrl G).

As shown in FIG. 10A a 3-fold and 5-fold TDP-43 knockdown was observed in SH-SY5Y cells using siTDP-43 RNA, when co-nucleofected with RNP comprising the Type V CRISPR nuclease variants of Nuclease D and Nuclease A, respectively. Knockdown of TDP-43 in the presence of a non-targeting control RNP complex resulted in a 120-fold and 130-fold increase in the STMN2 exon 2A splice variant when co-nucleofected with RNPs comprising the Type V CRISPR nuclease variants of Nuclease D and Nuclease A, respectively (FIG. 10B). An 8-fold and 10-fold decrease in the STMN2 full length transcript was observed with siTDP-43 knockdown and RNPs comprising the Type V CRISPR nuclease variant of Nuclease D and Nuclease A, respectively. (FIG. 10C). The exon 2A increase was significantly mitigated by RNA guide A_STMN2_Splice2a_4 (FIG. 10B). Significant restoration of the decreased full length STMN2 signal, due to TDP-43 depletion, was observed with RNA guide A_STMN2_Splice2a_4 (FIG. 10C).

### Example 7—Targeting STMN2 Exon 2a Splice Site by Cas12i2 in Human iPSC-Derived Motor Neurons

This Example shows that using Cas12i2 and RNA guides designed to disrupt the exon 2a splice site of STMN2 (the 3′ splice site in intron 1 as disclosed herein) results in a reduction of the STMN2 exon 2a splice variant and a corresponding increase in full length STMN2 transcripts.

Human iPSC-derived motor neurons (BrainXell) where thawed and cultured according to the protocol from the manufacturer. Briefly, motor neurons were thawed from liquid nitrogen very quickly in a 37 C water bath. The cells were added to medium containing 1:1 DMEM/F12 (Thermo Fisher #11330-032): Neurobasal medium (Life Technologies #21103-049), supplemented with 1×B27 (Thermo Fisher #17504-044), 1×N2 (Thermo Fisher #17502-048), 0.5 mM GlutaMAX (Thermo Fisher #35050-061), 10 ng/ml BDNF (Peprotech #450-02), ng/ml GDNF (Peprotech #450-10), 1 ng/ml TGF-β1 (Peprotech #100-21C), 15 μg/ml Geltrex (Life Technologies #A1413201), and 1× Motor Neuron Seeding Supplement (BrainXell). Next, the cells were subjected to trypan blue viability count, and plated onto plates previously coated with Poly-D-Lysine (Thermo Fisher #A3890401). Four days after plating, neurons were transduced with Cas12i2 (SEQ ID NO: 3) and RNA guide lentiviruses, each one at MOI 10. After 72 hours, neurons were also transduced with TDP43 shRNA lentivirus (VectorBuilder hTARDBP shRNA [AGATCTTAAGACTGGTCATTCCTCGAGGAATGACCAGTCTTAA GATCT; SEQ ID NO: 261]) or non-targeting shRNA (VectorBuilder Scramble shRNA [CCTAAGGTTAAGTCGCCCTCGCTCGAGCGAGGGCGACTTAACCTTAGG; SEQ ID NO: 262]) at MOI 10.

Ten days after transduction with nuclease and RNA guide (14 days after thawing), neurons were harvested for downstream RNA analysis and for NGS. The cells were rinsed with PBS and lysed with QuickExtract™ (DNA extraction solution; Lucigen) or RLT Plus (Qiagen). Cells in QuickExtract were incubated at 65° C. for 15 minutes, 68° C. for 15 minutes, and 98° C. for 10 minutes, before being analyzed by NGS. Samples for NGS were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were done with a 300 Cycle NEXTSEQ™ (Illumina) 500/550 High Output v2.5 Kit.

For cells in RLT Plus, RNA isolation was performed using QIAshredder columns and RNeasy plus Micro Kit (Qiagen). RNA was converted to cDNA using High-Capacity cDNA Reverse Transcription kit (Applied Biosystems). Quantitative reverse transcription PCR was performed using Faststart Universal Probe Master (Rox), (Roche), and TaqMan probes for TARDP (Hs00606522_m1, Thermo Fisher), STMN2 (Hs00975900_m1, Thermo Fisher) and GAPDH (Hs02786624_g1, Thermo Fisher). A TaqMan probe and PCR primers were used for detecting the STMN2 exon 2a splice variant.

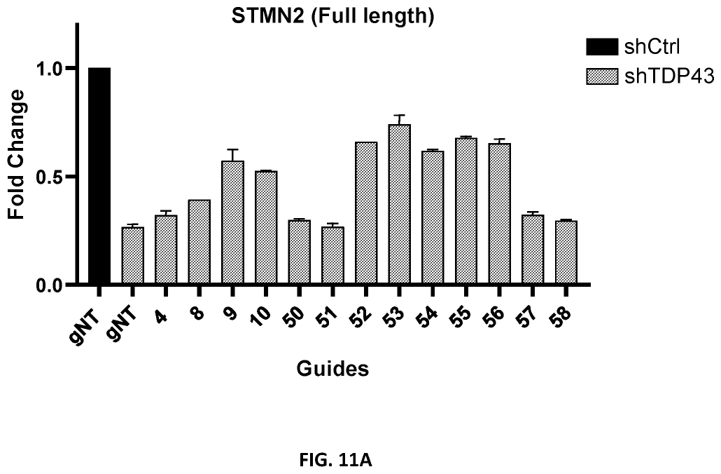

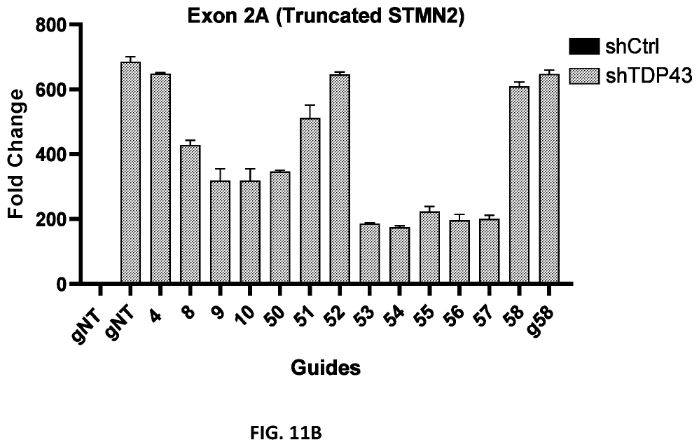

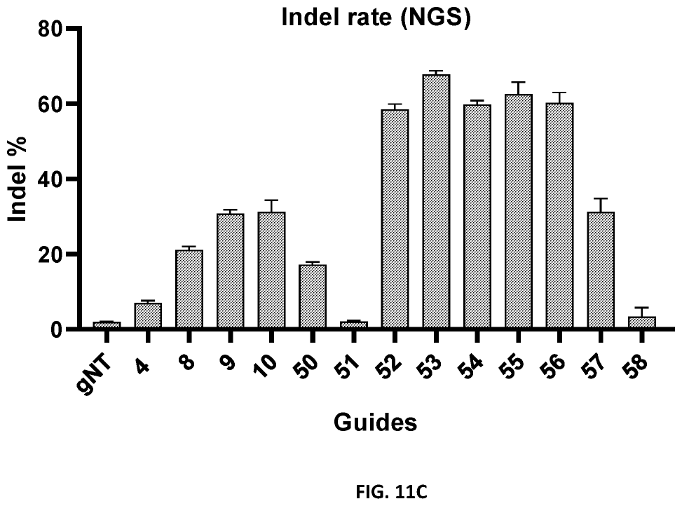

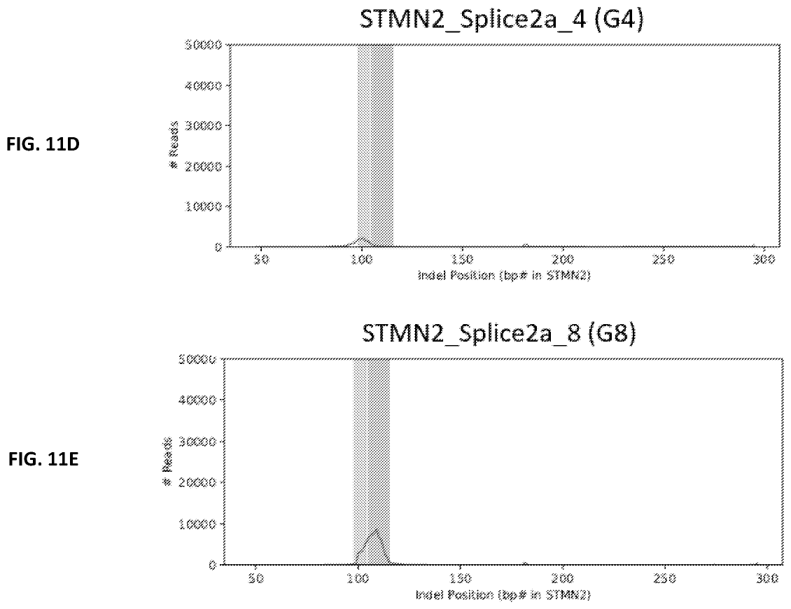

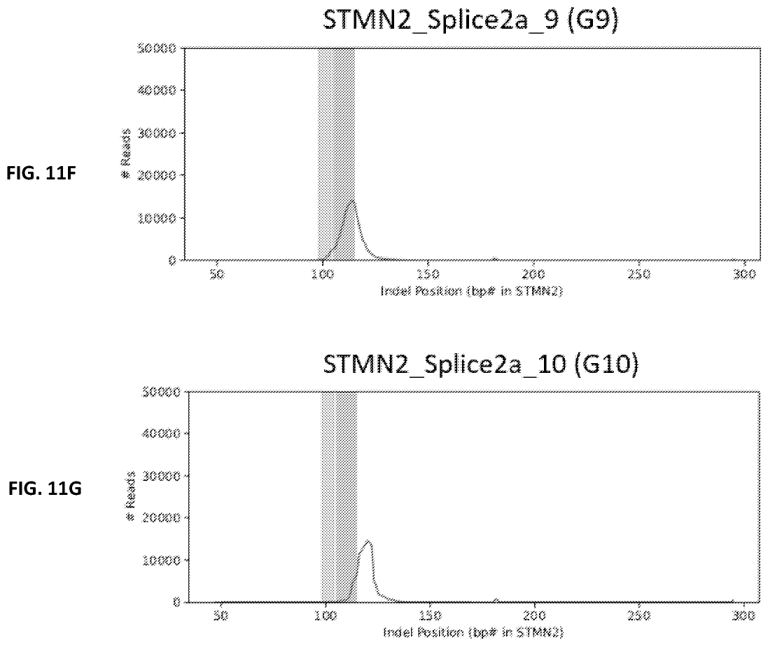

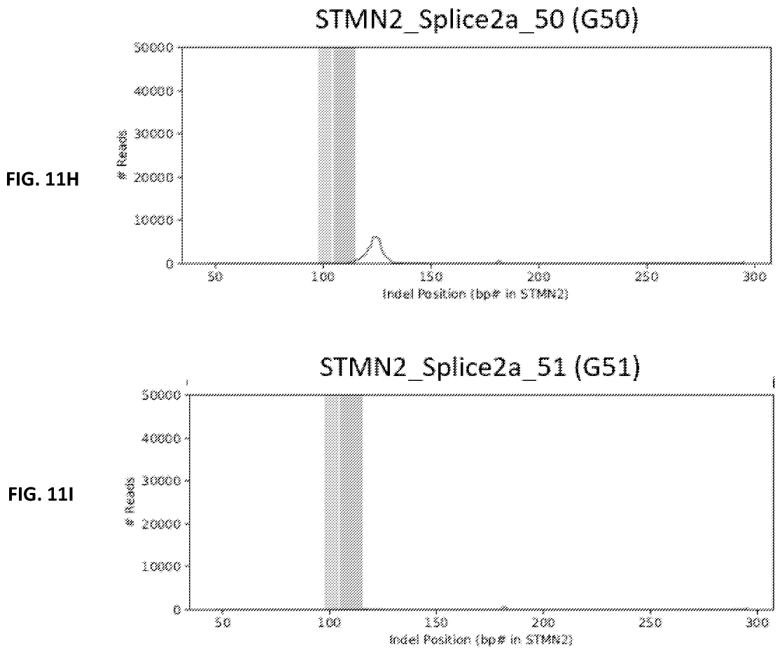

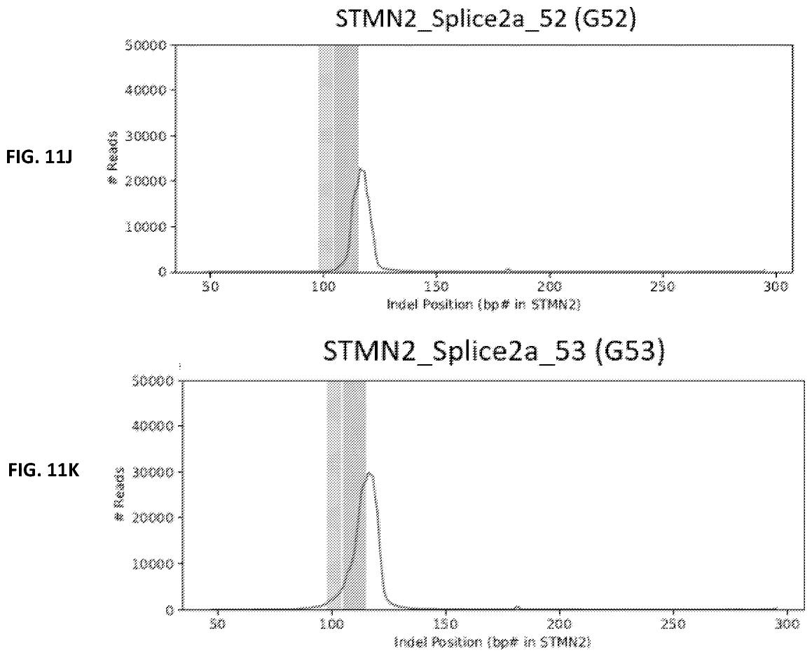

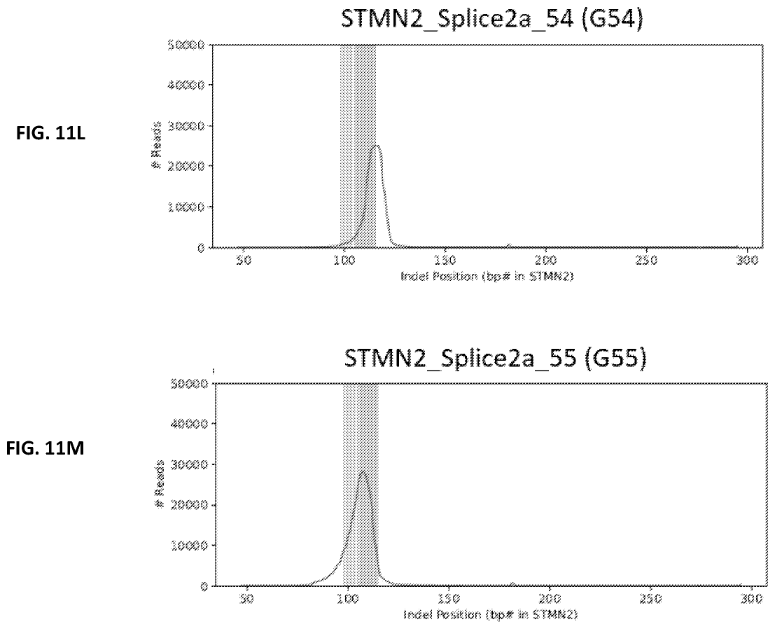

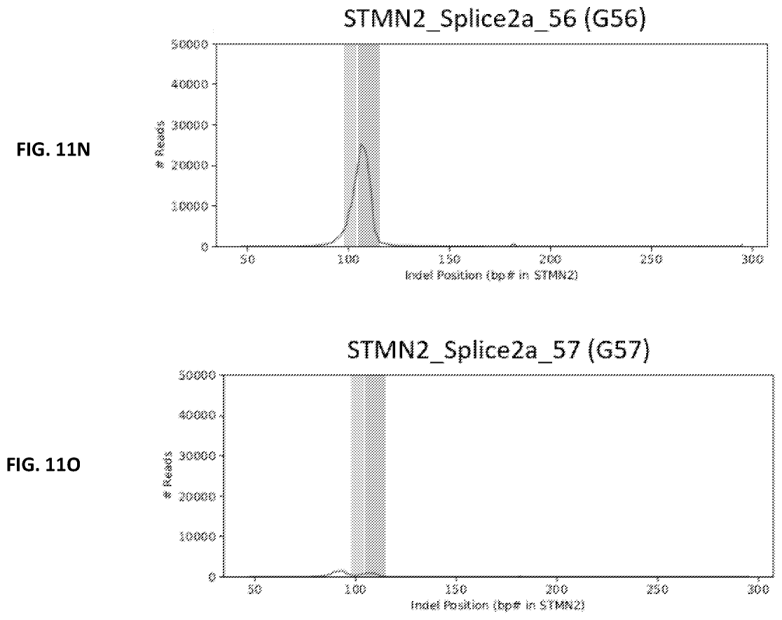

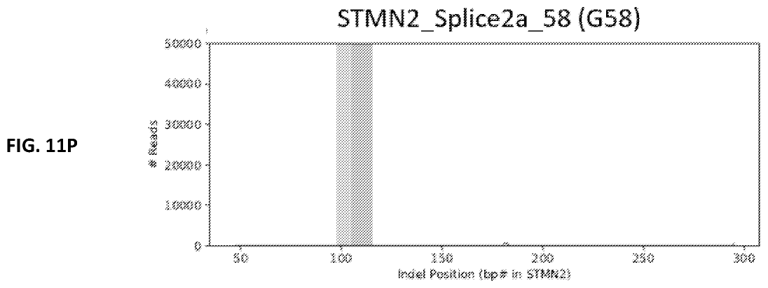

Knockdown of TDP-43 in the presence of a non-targeting control guide RNA resulted in a 75-fold decrease in the STMN2 full length transcript and around 700-fold increase in the STMN2 exon 2a splice variant (FIG. 11A and FIG. 11B). The exon 2a increase was partially mitigated by RNA guides G9, G10, and, more markedly by G52, G53, G54, G55, and G56 (FIG. 11B). An inverse relationship was observed between exon 2a and full length Stathmin-2 RNA, with a significant restoration of the decreased full length STMN2 signal observed with the same RNA guides (FIG. 11A).

FIG. 11C shows indel activity of the tested RNA guides in motor neurons. It was observed a correlation between guides that promoted STMN2 full length recovery and raw indels. Guides G53 and G55 showed the highest indels rates of 67.7% and 62.5%, respectively.

FIGS. 11D-11P show the positions of indels induced by a particular guide as indicated in association with Cas12i2 in motor neurons within the STMN2 amplicon (x-axis).

Therefore, this Example shows that in the presence of TDP-43 knockdown, editing with Cas12i2 and RNA guides targeting the STMN2 exon 2a splice site not only disrupts the DNA splice site and optimal disruption window, resulting in a decrease in the exon 2a splice variant, but also promotes a corresponding increase in full length STMN2.

### Example 8—Targeting STMN2 Exon 2a Splice Site by Nuclease A in Human iPSC-Derived Motor Neurons

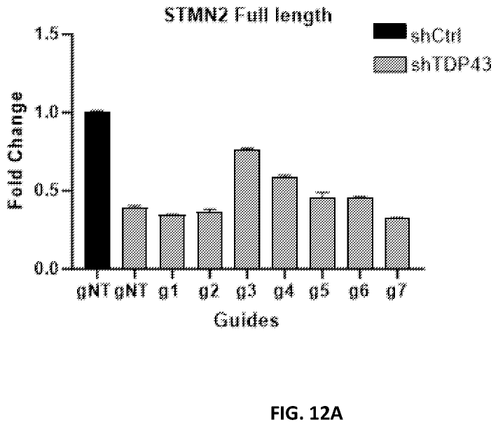

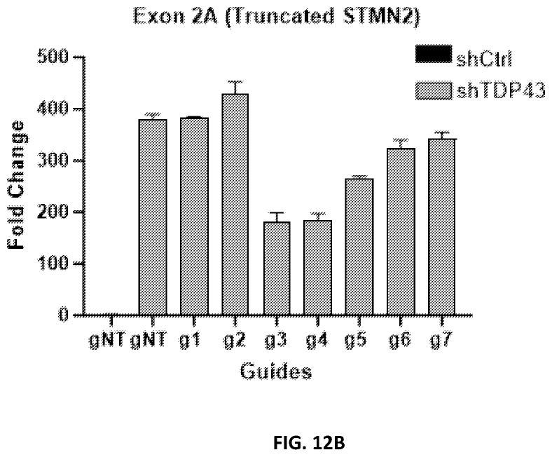

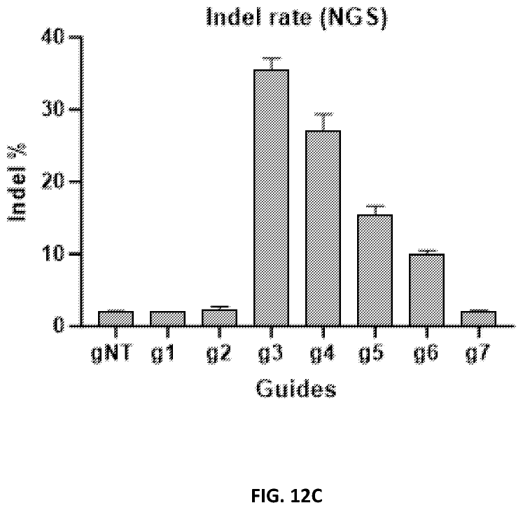

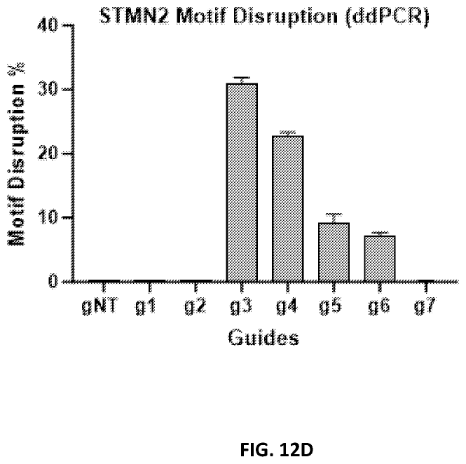

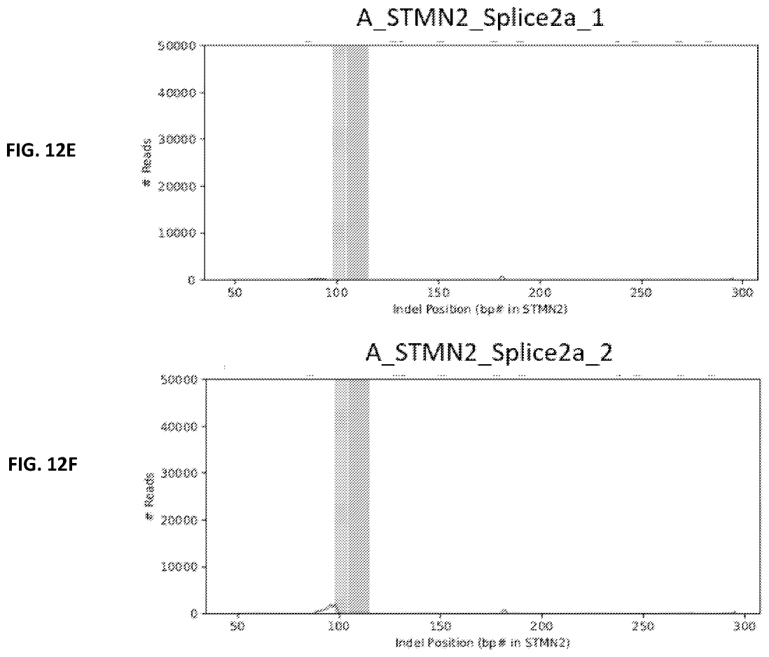

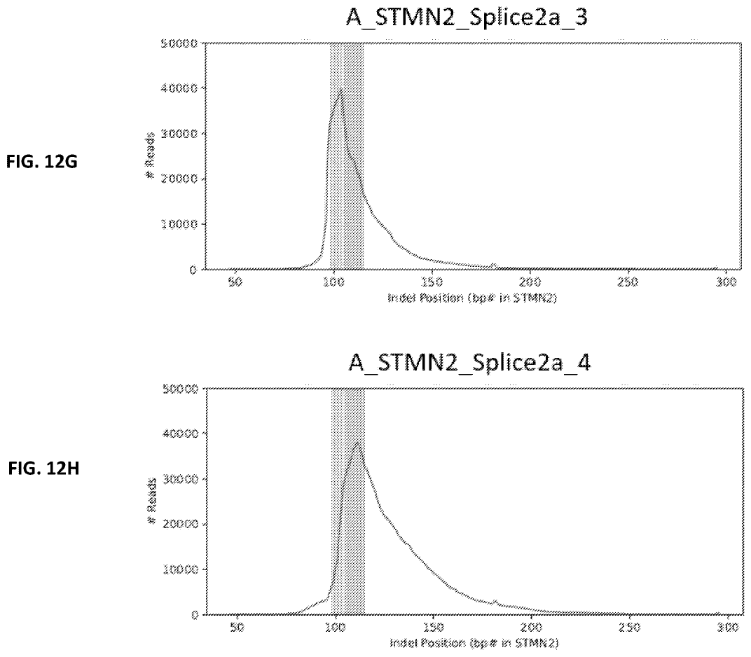

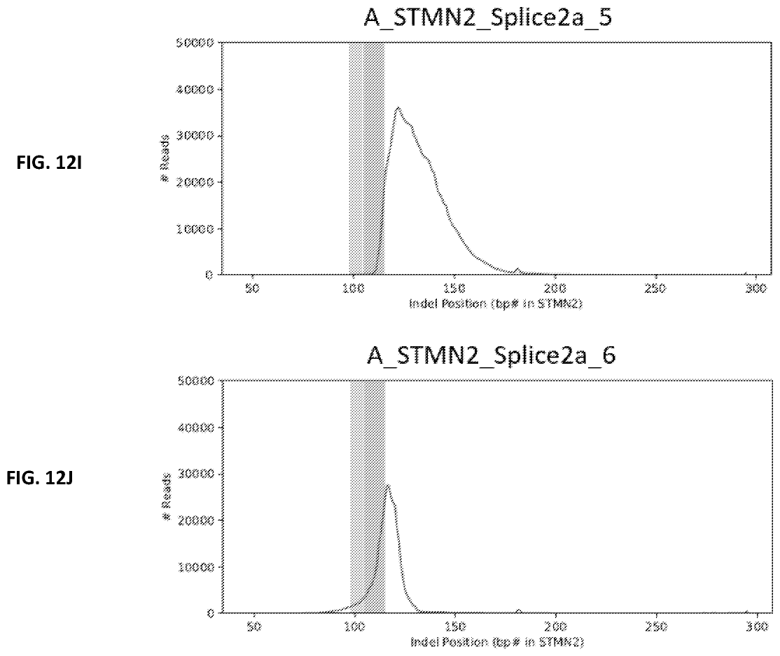

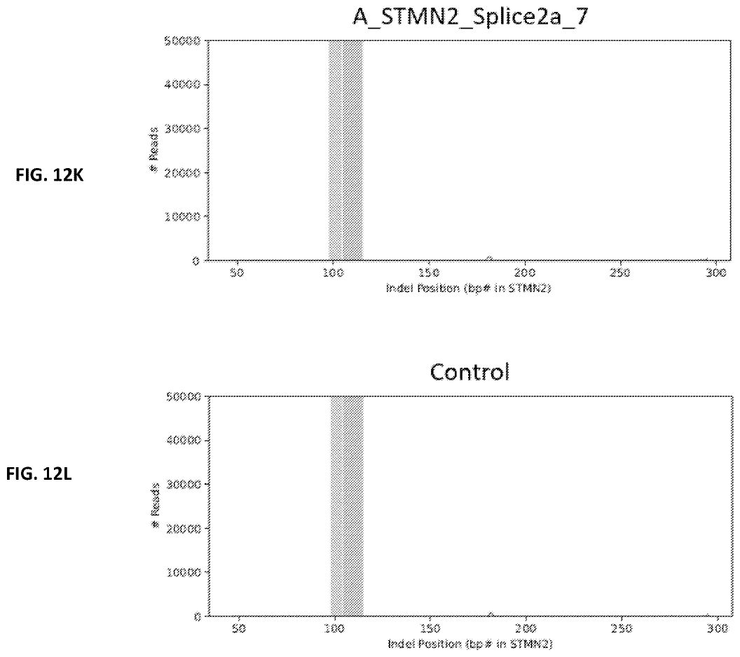

This Example shows that using a Type V nuclease (Nuclease A listed in Table 1 above) complexed with an RNA guide designed to disrupt the exon 2a splice site of STMN2 (the 3′ splice site in intron 1 as disclosed herein) results in a reduction of the STMN2 exon 2a splice variant and a corresponding increase in full length STMN2 transcripts in motor neurons. Human iPSC-derived motor neurons (BrainXell) where thawed and cultured according to the protocol from the manufacturer. Briefly, motor neurons were thawed and added to medium containing 1:1 DMEM/F12 (Thermo Fisher #11330-032): Neurobasal medium (Life Technologies #21103-049), supplemented with 1×B27 (Thermo Fisher #17504-044), 1×N2 (Thermo Fisher #17502-048), 0.5 mM GlutaMAX (Thermo Fisher #35050-061), 10 ng/ml BDNF (Peprotech #450-02), 10 ng/ml GDNF (Peprotech #450-10), 1 ng/ml TGF-β1 (Peprotech #100-21C), 15 μg/ml Geltrex (Life Technologies #A1413201), and 1×Motor Neuron Seeding Supplement (BrainXell). Motor neurons were transduced first with Nuclease A (SEQ ID NO: 4) and RNA guide lentiviruses, and further transduced with TDP43 shRNA lentivirus (VectorBuilder hTARDBP shRNA or non-targeting shRNA (VectorBuilder Scramble shRNA. The RNA guides and the corresponding target sequences are provided in Table 3 above, for reference to the sample identifiers, see, FIGS. 12A-12D, see, Table 7.

Ten days after transduction with nuclease and RNA guide, neurons were harvested for downstream RNA analysis and for NGS. The cells were rinsed with PBS and lysed with QuickExtract™ (DNA extraction solution; Lucigen) or RLT Plus (Qiagen). Samples for NGS were prepared by two rounds of PCR. The first round (PCR1) was used to amplify specific genomic regions depending on the target. Round 2 PCR (PCR2) was performed to add Illumina adapters and indices. Reactions were then pooled and purified by column purification. Sequencing runs were done with a 300 Cycle NEXTSEQ™ (Illumina) 500/550 High Output v2.5 Kit.

For cells in RLT Plus, RNA isolation was performed using QIAshredder columns and RNeasy plus Micro Kit (Qiagen). RNA was converted to cDNA using High-Capacity cDNA Reverse Transcription kit (Applied Biosystems). Quantitative reverse transcription PCR was performed using Faststart Universal Probe Master (Rox), (Roche), and TaqMan probes for TARDP (Hs00606522_m1, Thermo Fisher), STMN2 (Hs00975900_m1, Thermo Fisher) and GAPDH (Hs02786624_g1, Thermo Fisher). A TaqMan probe and PCR primers were used for detecting the STMN2 exon 2a splice variant.

Knockdown of TDP-43 in the presence of a non-targeting control guide RNA resulted in a 60-fold decrease in the STMN2 full length transcript and around 380-fold increase in the STMN2 exon 2a splice variant (FIG. 12A and FIG. 12B). The exon 2a increase was markedly mitigated by RNA guides G3 and G4 (FIG. 12B). An inverse relationship was observed between exon 2a and full length Stathmin-2 RNA, with a partial restoration of the decreased full length STMN2 signal observed with the same RNA guides (FIG. 12A).

FIG. 12C shows indel activity of the tested RNA guides in motor neurons. It was observed a correlation between guides that promoted STMN2 full length recovery and raw indels. Guides G3 and G4 showed the highest indels rates of 35% and 27%, respectively.

FIG. 12D illustrates STMN2 motif disruption in motor neurons analyzed by a digital droplet polymerase chain reaction (ddPCR) assay.

FIGS. 12E-12L include diagrams showing the positions of indels induced by a particular guide as indicated in association with Nuclease A in motor neurons within the STMN2 amplicon (x-axis).

Therefore, this Example shows that in the presence of TDP-43 knockdown, editing with Nuclease A and RNA guides targeting the STMN2 exon 2a splice site not only disrupts the DNA splice site, resulting in a decrease in exon 2a-containing splice variant, but also promotes a corresponding increase in full length STMN2.

### Example 9—Reversal of Phenotypic Deficits Due to STMN2 Mis-Splicing in Human iPSC-Derived Motor Neurons

This Example shows that using Cas12i2 (SEQ ID NO: 3) and an RNA guide (STMN2-Exon2a-55, SEQ ID NO: 31 (G55)), designed to disrupt the exon 2a splice site of STMN2 (the 3′ splice site in intron 1 as disclosed herein), results in the phenotypic rescue of the abnormal neuronal outgrowth promoted by TDP-43 knockdown in human iPSC-derived motor neurons.

Human iPSC-derived motor neurons were generated as described in Du et al. 2015. On day 20-22 after starting the iPSC differentiation, neurons were transduced with Cas12i2 (SEQ ID NO: 3) and RNA guide lentiviruses. After 72 hours, neurons were also transduced with TDP43 shRNA lentivirus or non-targeting shRNA.

Twenty-five days after transduction with nuclease and RNA guide, neurons were fixed, blocked, permeabilized, and incubated with primary antibodies overnight at 4 C (rabbit anti-STMN2, Abcam, #ab185956, 1:500; mouse anti-β3-Tubuli, R&D Systems, #MAB1195, 1:1,000). Cells were then washed with PBS incubated with secondary antibodies (Alexa Fluor 488 and 555, Life Technologies, 1:1000) at room temperature. Nuclei were stained using Hoechst 33342 (1 μg/ml). Images were captured and analysed using the Operetta CLS High-Content Analysis System (Perkin Elmer).

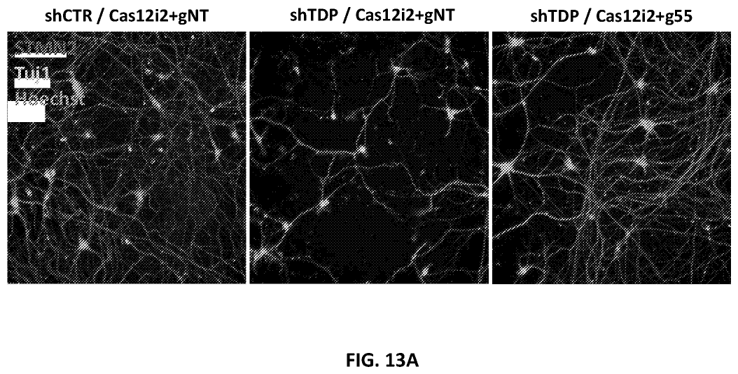

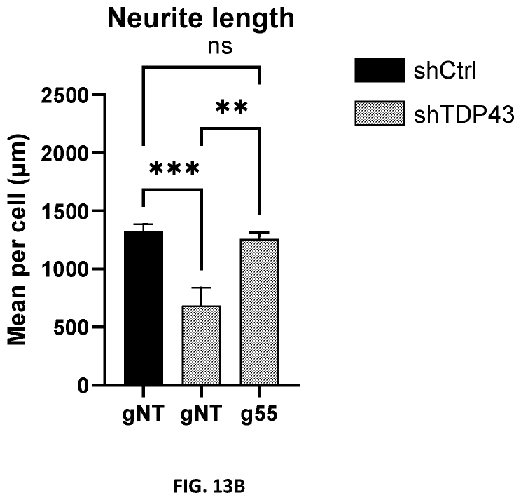

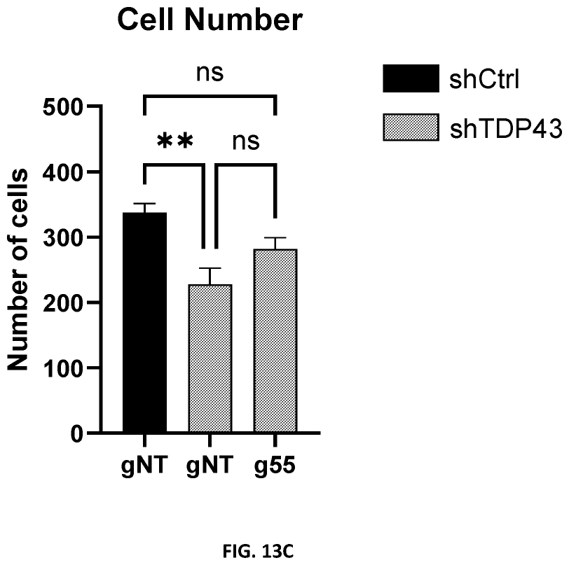

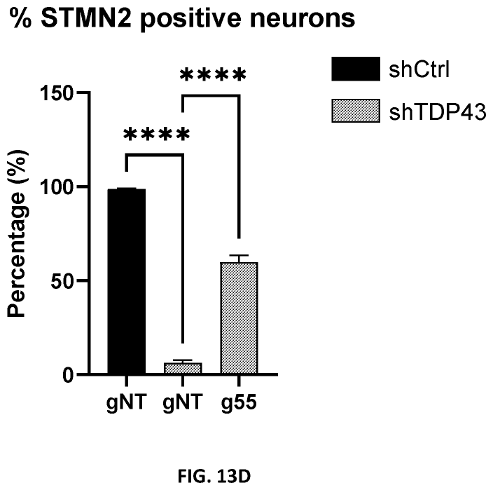

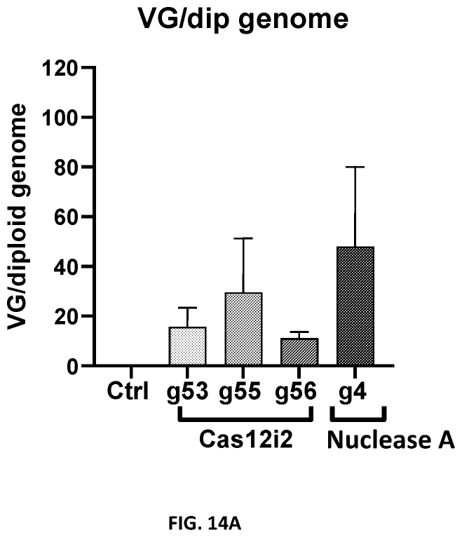

Knockdown of TDP-43 in the presence of a guide RNA that does not target the STMN2 intron 3′ splice site targeting guide RNA (non-targeting control) resulted in approximately 50% reduction in the motor neuron neurite outgrowth (FIG. 13A middle panel, and FIG. 13B), determined by measuring the length of Tuj1-positive neurites. This impairment was dramatically reversed in the presence of RNA guide G55 (FIG. 13A right panel, and FIG. 13B). Although no marked change was observed in the cell number after treatment with RNA guide G55 (FIG. 13C), a significant increase in the percentage of STMN2-positive neurons was observed (FIG. 13D), from 6% STMN2-positive neurons in the non-targeting guide shTDP43 condition to 60% STMN2-positive neurons with G55 and TDP43 knockdown.

Therefore, this Example shows that in the presence of TDP-43 knockdown, editing with Cas12i2 and RNA guide G55, targeting the STMN2 exon 2a splice site and optimal disruption window, not only disrupts the DNA splice site resulting in a reversal of the molecular phenotypes (Example 7), but also promotes a corresponding phenotypic rescue by reversing the compromised neurite outgrowth and increasing the number of STMN2-positive neurons.

### Example 10—Gene Editing Reduces the Aberrant Splicing of STMN2 in Exon 2a Humanized Mice

This Example demonstrates that Cas12i2 and Type V nucleases and corresponding RNA guides designed to disrupt the exon 2a splice site of STMN2 (the 3′ splice site in intron 1 as disclosed herein) reduce the aberrant splicing of STMN2 in exon 2A humanized mice.

Stmn2^{em8(STMN2*)} mice are provided by The Jacksons Laboratories (Jax) (RRID: MMRRC_069792-JAX: on the world wide web at: jax.org/strain/035721). In these mice, the STMN2-like gene was CRISPR/Cas9 engineered to generate a mutant carrying 222 nucleotides of human STMN2 exon 2a. In addition, the STMN2 sequence was modified to include the human MS2 stem loop sequence and replace the TDP43 binding site. Animal experiments were performed at Jax. Briefly, at the age of post-gestational day 1 (P1), mouse pups received bilateral ICV injections of 2.5 μl of vector into each lateral ventricle (2×10¹¹ AAV vector genomes per injection). Sample Identifiers and Corresponding Nucleases and RNA guides are shown in Table 8.

TABLE 8
Sample Identifiers and Corresponding Nucleases and RNA Guides

| Group | | SEQ | | SEQ |
|---|---|---|---|---|
| Control | | ID | | ID |
| (Ctrl) | Nuclease | NO | gRNA | NO |
| 1 (g53) | Cas12i2 | 266 | STMN2-Exon2a- | 29 |
| | | | 53(G53) | |
| 2 (g55) | | | STMN2-Exon2a- | 31 |
| | | | 53(G55) | |
| 3 (g56) | | | STMN2-Exon2a- | 32 |
| | | | 56(G56) | |
| 4 (g4) | Nuclease A | 4 | A_STMN2_Splice2a_4 | 52 |

The AAV genome expressed the Cas12i2 (SEQ ID NO: 266) under the control of the synapsin promoter and express the gRNA under the control of a U6 promoter.

Twenty-eight days post-injection, animals were sacrificed. The brain was removed and sectioned in the sagittal plane to separate the right and left hemispheres; the cerebellum was removed. Frozen mouse hemibrains were homogenized using a TissueLyser II (Qiagen) in RLT Plus buffer (Qiagen) containing beta-mecaptoethanol (Sigma Aldrich). DNA and RNA were isolated using the All Prep Universal kit (Qiagen). RNA was converted to cDNA using High-Capacity cDNA Reverse Transcription kit (Applied Biosystems). Quantitative reverse transcription PCR was performed using Faststart Universal Probe Master (Rox) (Roche), and TaqMan probes were used to detect full length STMN2 full-length (IDT Stock Assay #Mm.PT.58.13787385) and the exon 2a splice variant. Primers/probes used for the detection of exon 2a were: Forward primer: GCCTTACTCAGACTCCTCTCTC (SEQ ID NO: 263), Reverse primer: TCTTCTGCCGAGTCCCATT (SEQ ID NO: 264), and Probe: CTGGACCCTTCTCCTTTGCCTTCG (SEQ ID NO: 265) (Baughn M W et al., Science, 2023 Mar. 17; 379 (6637): 1140-1149).

DNA was analyzed by a digital droplet polymerase chain reaction (ddPCR) assay to determine the efficiency of nuclease cutting at defined STMN2 splice site. The assay utilizes the compartmentalization of genomic DNA into oil-like droplets, followed by amplification and fluorescent detection, to quantify number of events with and without STMN2 disruption.

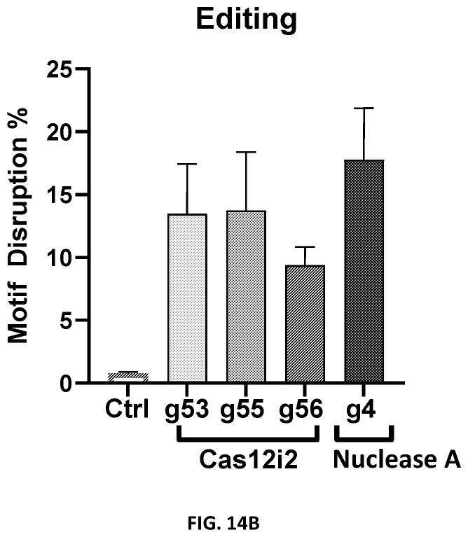

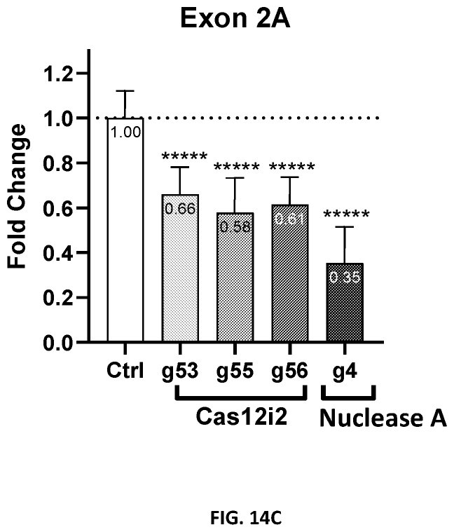

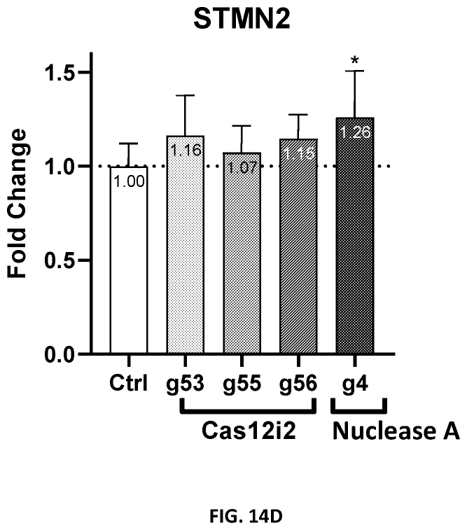

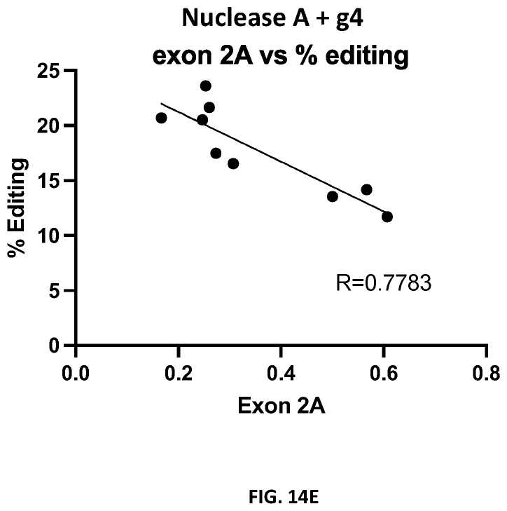

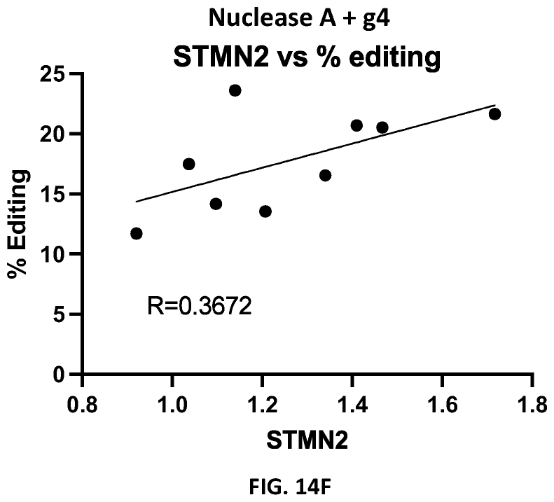

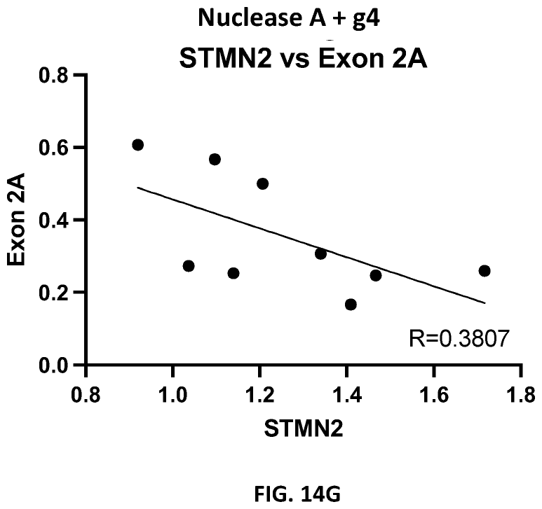

The motif disruption observed in vivo after injection with each vector tested is shown in FIG. 14B. The number of vector genomes per diploid genome (VG/dg) was determined relative to copies of bovine growth hormone (bGH) and can be found in FIG. 14A. The highest motif disruption was observed in animals injected with Nuclease A+RNA guide 4 (A_STMN2_Splice2a_4), followed by animals injected with Cas12i2+g55, Cas12i2+g53, and Cas12i2+g56 (average of 17.8%, 13.8%, 13.5%, and 9.4%, respectively). The exon 2a levels observed in animals injected with vehicle (Ctrl) had a significant reduction after treatment with all vectors, being more pronounced in animals that received Nuclease A+RNA guide 4 (65% reduction; FIG. 14C). In these mice, a significant increase of full-length Stathmin-2 RNA was also observed (26% increase; FIG. 14D).

The correlations between editing percentage and STMN2 transcript levels in mice injected with Nuclease A+g4 are shown in FIGS. 14E-14G.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A gene editing system, comprising:
(a) a Type V CRISPR nuclease or a nucleic acid encoding the nuclease; and
(b) one or more guide RNAs (gRNAs) targeting a Stathmin-2 (STMN2) gene, or one or more nucleic acids encoding the one or more gRNAs;
wherein the gene editing system leads to (a) a deletion of one or more nucleotides in a 3′ splice site of intron 1 of STMN2, wherein the 3′ splice site is adjacent to exon 2a; (b) a deletion of one or more nucleotides in a region of intron 1 that is adjacent to the 3′ splice site, or both (a) and (b), thereby reducing production of STMN2 transcripts including exon 2a and increasing production of functional STMN2 transcripts in a cell edited by the gene editing system.

2. The gene editing system of claim 1, wherein the Type V CRISPR nuclease is a Cas12i2 nuclease, which optionally comprises an amino acid sequence at least 90% identical to SEQ ID NO: 3.

3. The gene editing system of claim 2, wherein the Type V CRISPR nuclease comprises the amino acid sequence of SEQ ID NO: 3 or SEQ ID NO: 266.

4. The gene editing system of claim 1, wherein the Type V CRISPR nuclease is a nuclease comprising the amino acid sequence of any one of SEQ ID NO: 4-6, or a variant thereof; optionally wherein the variant comprises an amino acid sequence at least 90% identical to SEQ ID NO: 4, 5, or 6.

5. The gene editing system of claim 3, wherein the Type V CRISPR nuclease comprises the amino acid sequence of SEQ ID NO: 4 or SEQ ID NO: 255.

6. The gene editing system of claim 1, wherein:
(a) the Type V CRISPR nuclease is a Cas12i2 nuclease, which optionally comprises an amino acid sequence at least 90% identical to SEQ ID NO: 3 or SEQ ID NO: 266; and
(b) the one or more guide RNAs (gRNAs) are selected from those listed in Table 2; optionally wherein the gRNA is G53, G55, or G56.

7. The gene editing system of claim 1, wherein:
(a) the Type V CRISPR nuclease is a nuclease comprising an amino acid sequence at least 90% identical to SEQ ID NO: 4; and
(b) the one or more guide RNAs (gRNAs) are selected from those listed in Table 3; optionally wherein the gRNA is A_STMN2_Splice2a_4 or A_STMN2_Splice2a_3.

8. The gene editing system of claim 1, wherein:
(a) the Type V CRISPR nuclease is a nuclease comprising an amino acid sequence at least 90% identical to SEQ ID NO: 5; and
(b) the one or more guide RNAs (gRNAs) are selected from those listed in Table 4.

9. The gene editing system of claim 1, wherein:
(a) the Type V CRISPR nuclease is a nuclease comprising an amino acid sequence at least 90% identical to SEQ ID NO: 6; and
(b) the guide RNA (gRNA) are selected from those listed in Table 5.

10. The gene editing system of claim 1, which comprises the nucleic acid encoding the Type V CRISPR nuclease.

11. The gene editing system of claim 10, wherein the nucleic acid is a vector, which comprises a first nucleotide sequence encoding the Type V CRISPR nuclease, the first nucleotide sequence being in operable linkage to a first promoter.

12. The gene editing system of claim 11, wherein the vector further comprises a second nucleotide sequence encoding the gRNA, the second nucleotide sequence being in operable linkage to a second promoter.

13. The gene editing system of claim 11, wherein the vector is an adeno-associated viral (AAV) vector, which optionally is an AAVrh10 vector.

14. The gene editing system of claim 11, wherein the first promoter is a synapsin 1 promoter.

15. A method for inhibiting aberrant splicing in a Stathmin-2 (STMN2) transcript, the method comprising:
(i) genetically editing a STMN2 gene in a cell to delete (a) one or more nucleotides in a 3′ splice site of intron 1, wherein the 3′ splice site is adjacent to exon 2a, (b) one or more nucleotides in a region of intron 1 that is adjacent to the 3′ splice site, or both (a) and (b),
thereby inhibiting production of STMN2 transcripts including exon 2a and improving production of functional STMN2 transcripts in the cell.

16. The method of claim 15, wherein the 3′ splice site in (a) comprises the nucleotide sequence of TTGCAG; and/or
wherein the region of exon 2a in (b) comprises the nucleotide sequence of ACTCGGCAGA (SEQ ID NO: 2).

17. The method of claim 15, wherein step (i) results in deletions in both (a) and (b).

18. The method of claim 15, wherein the genetic editing step (i) is mediated by a gene editing system.

19. The method of claim 18, wherein the gene editing system comprises (i) a Type V CRISPR nuclease or a first nucleic acid encoding the nuclease, and (ii) a guide RNA (gRNA) targeting the STMN2 gene or a second nucleic acid encoding the gRNA.

20. The method of claim 19, wherein the Type V CRISPR nuclease is a Cas12i2 nuclease, which optionally comprises an amino acid sequence at least 90% identical to SEQ ID NO: 3.

21. The method of claim 15, wherein the cell is in cell culture; optionally wherein the cell is from a human patient having amyotrophic lateral sclerosis (ALS) or frontotemporal dementia (FTD).

22. The method of claim 21, wherein the method further comprises measuring levels of STMN2 transcripts and/or STMN2 protein in the cell after the genetic editing.

23. The method of claim 15, wherein the cell is a brain cell, which optionally is a neuronal cell.

24. The method of claim 23, wherein the cell is a motor neuronal cell.

25. The method of claim 23, wherein the neuronal cell is in a human patient having amyotrophic lateral sclerosis (ALS) or frontotemporal dementia (FTD).

26. The method of claim 25, comprising delivering the gene editing system to a subject in need thereof; wherein the gene editing system comprises:
(a) a Type V CRISPR nuclease or a nucleic acid encoding the nuclease; and
(b) one or more guide RNAs (gRNAs) targeting a Stathmin-2 (STMN2) gene, or one or more nucleic acids encoding the one or more gRNAs; and
wherein the gene editing system leads to (a) a deletion of one or more nucleotides in a 3′ splice site of intron 1 of STMN2, wherein the 3′ splice site is adjacent to exon 2a; (b) a deletion of one or more nucleotides in a region of intron 1 that is adjacent to the 3′ splice site, or both (a) and (b), thereby reducing production of STMN2 transcripts including exon 2a and increasing production of functional STMN2 transcripts in a cell edited by the gene editing system.

27. The method of claim 26, wherein the gene editing system is delivered to the subject by intracerebroventricular (ICV) injection or intrathecal injection.

28. A genetically edited cell, comprising (a) a deletion of one or more nucleotides in a 3′ splice site of intron 1 of STMN2, wherein the 3′ splice site is adjacent to exon 2a; (b) a deletion of one or more nucleotides in a region of intron 1 that is adjacent to the 3′ splice site, or both (a) and (b), wherein the genetically edited cell produces a reduced level of STMN2 transcripts including exon 2a and an increased level of functional STMN2 transcripts as compared with a non-edited counterpart.

29. A gene editing system, comprising:
(a) a Type V CRISPR nuclease comprising an amino acid sequence at least 90% identical to SEQ ID NO: 4 or a first nucleic acid encoding the Type V CRISPR nuclease; and
(b) a guide RNA (gRNA) targeting a Stathmin-2 (STMN2) gene or a second nucleic acid encoding the gRNA;
wherein the gene editing system genetically modifies the STMN2 gene to inhibit production of STMN2 transcripts containing exon 2a.

30. The gene editing system of claim 29, wherein the Type V CRISPR comprises the amino acid sequence of SEQ ID NO: 4 or SEQ ID NO: 255.

31. The gene editing system of claim 29, which comprises the first nucleic acid encoding the Type V CRISPR nuclease.

32. The gene editing system of claim 31, wherein the nucleic acid is a vector, which comprises a first nucleotide sequence encoding the Type V CRISPR nuclease, the first nucleotide sequence being in operable linkage to a first promoter.

33. The gene editing system of claim 32, wherein the vector further comprises a second nucleotide sequence encoding the gRNA, the second nucleotide sequence being in operable linkage to a second promoter.

34. The gene editing system of claim 32, wherein the vector is an adeno-associated viral (AAV) vector, which optionally is an AAVrh10 vector.

35. The gene editing system of claim 32, wherein the first promoter is a synapsin 1 promoter.

36. A method for genetically editing a Stathmin-2 (STMN2) gene, comprising contacting cells with the gene editing system set forth in claim 29 to allow for genetic editing of the STMN2 gene in the cells by the gene editing system.

37. The method of claim 36, wherein the contacting step is performed by administering the gene editing system to a subject in need thereof.

38. The method of claim 37, wherein the gene editing system is delivered to the subject by intracerebroventricular (ICV) injection or intrathecal injection.

39. A method for treating a disease involving aberrant splicing of STMN2, the method comprising administering to a subject in need thereof an effective amount of a gene editing system set forth in claim 1.

40. The method of claim 39, wherein the subject is a human patient.

41. The method of claim 39, wherein the disease is ALS or FTD.


SEQUENCE LISTING (271 sequences)

SEQ ID NO: 1 - DNA, 445 bp, Synthetic construct
  misc_feature 1..445: note = synthetic Polynucleotide
  source 1..445: mol_type = other DNA
  polyA_site 308..313: note = premature poly(A) site ATTAAA
  intron 106..115: note = ACTCGGCAGA
acttccgaactcatatacctggggattttattactctgggaattatgtgttctgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatcttatatttattatttaccaaattattctaagagtatttcttcctgaataccatgtgagaaaattcttaagaatttattgagtatgactgtatatttgaaaagagtg

SEQ ID NO: 2 - DNA, 10 bp, Synthetic construct
  misc_feature 1..10: note = synthetic Polynucleotide
  source 1..10: mol_type = other DNA
actcggcaga

SEQ ID NO: 3 - protein, 1054 aa, Synthetic construct
  REGION 1..1054: note = synthetic polypeptide
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 4 - protein, 745 aa, Synthetic construct
  REGION 1..745: note = synthetic polypeptide
MIKSIQLKVKGECRITKDVINEYKEYYNNCSRWIKNNLTSITIGEMAKFLQSLSDKEVAYRSMGLSDEWKDKPLYHLFTKKYHTKNADNLLYYYIKEKNLDGYKGNTLNISNTSFRQFGYFKLVVSNYRTKIRTLNCKIKRKKIDADSTSEDIEMQVMYEIIKYSLNKKSDWDNFISYIENVENPNIDNINRYKLLRECFCENENMIKNKLELLSVEQLKKFGGCIMKPHINSMTINIQDFKIEEKENSLGFILHLPLNKKQYQIELLGNRQIKKGTKEIHETLVDITNTHGENIVFTIKNDNLYIVFSYRSEFEKEEVNFAKTVGLDVNFKHAFFVGSEKDNCHLDGYINLYKYLLEHDEFTNLLTEDERKDYEELSKVVTFCPFENQLLFARYNKMSKFCKKEQVLSKLLYALQKKLKDENRTKEYIYVSCVNKLRAKYVSYFILKEKYYEKQKEYDIEMGFVDDSTESKESMDKRRTEYPFRNTPVANELLSKLNNVQQDINGCLKNIINYIYKIFEQNGYKVVALENLENSNFEKKQVLPTIKSLLKYHKLENQNVNDIKASDKVKEYIENGYYELMTNENNEIVDAKYTEKGAMKVKNANFFNLMMKSLHFASVKDEFVLLSNNGKTQIALVPSEFTSQMDSTDHCLYMKKNDKGKLVKADKKEVRTKQERHINGLNADFNAANNIKYIVENEVWRGIFCTRPKKTEYNVPSLDTTKKGPSAILNMLKKIGAIKVLETEK

SEQ ID NO: 5 - protein, 756 aa, Synthetic construct
  REGION 1..756: note = synthetic polypeptide
MTTKQVKSIVLKVKNTNECPITKDVINEYKKYYNICSEWIKDNLTSITIGDIASFLKEARNKDTIPTYINMGLSEEWKYKPIYHLFTDRYHEKSANNLLYAYFKEKNLDCYNGNILNLSETYYRRNGYFKSVVGNYRTKIRTLNYKIKRKNVDENSTNEDIELQVMYEIAKRKLNIKKDWENYISYIENVENINIKNIDRYNLLYKHFCENESTINCKMELLSVEQLKEFGGCVMKQHINSMTINIQDFKIENKENSLGFILNLPLNKKKYQIELWGNRQIKKGNKDNYKTLVDFINTYGQNIIFTIKNNKIYVVFSYECELKEKEINFDKIVGIDVNFKHALFVASERDKNPLQGNNQLKGYINLYGYLLEHNEFTSLLTKEELDIYKEIAKGVTFCPLEYNLLFTRIENKGGKSNDKEQVLSKLLYSLQIKLKNENKIQEYIYVSCVNKLRAKYVSYFILKEKYYEKQKEYDIEMGFTDDSTESKESMDKRRLEFPFRNTQIANGFLEKLSNVQQDINGCLKNIINYAYKVFEQNGFGVIALENLENSNFEKTQVLPTIKSLLRYHKLRNQNINNINASDKVKEYIEKEYYELTTNENNEIVDAKYTKKGIIKVKKANFFNLMMKSLHFASNKDEFILLSNNGKTQIALVPSEYTSQMDSIEHCLYVDKNGKKVDKKKVRQKQETHINGLNADFNAANNIKYIIENENLRKLFCGKLKVSGYNTPILRATKKGQFNILAELKKQNKIKIFEIEK

SEQ ID NO: 6 - protein, 654 aa, Synthetic construct
  REGION 1..654: note = synthetic polypeptide
MGAARRRNPKVAAARKGKPPPKATGNCRNYRYGAHEPIANLDKVLDEMRGAHDLRNVLTCINRARSEMITAALGEHQSYKKATADLAALHQRRDKLEAQIRQQNSASRKRLGRHSPLSSELDTVRKRIDEGRTALKKLRRKLLKKDPALKAVVEAADDMAKRETTRAEDACGLYWCTRNEQTGKRAKLRRFKKWRDSEATISVQIPGGLTVEQLLGGENNQARLELRPEGVWVQGARKRKVEPAEAARNKLRLDEDGYPMRKLGTAILHLRCMSDEDGKPIWAEVPITYHREIPADAKIKRCYLHRFRVGNRYHWSVRFSLERGKKGDDSWLHPRVATTGTAAIDIGWRWFPDRLRVAVWAGSDGAEGELCLPKWWLDEMYSVRLDQRERDVLFNEIVSLVLPWFRSRRGELSDYVVQAIKTMHSWRDKGRLAALSMRWRDDLAADPGANPAHVAMSIRLEEWRKRDKHIWCEEVNLRSQLQGSRKDLYRRFAAMLTSRYGRIVVEEFRLSAVQKLPPASIDDGTYSRVKRHKGDAACSHLVGALKDAARQLDKKNPKWTTKRCHVCGKTERKWENPGELEHTCKHCGVLWDRDVNAARNILAASGVAVDWTRPPLAPAARMTYPQVENREMRRSRRRKEALETTRASGDRQTA

SEQ ID NO: 7 - RNA, 19 nt, Synthetic construct
  misc_feature 1..19: note = synthetic Polynucleotide
  source 1..19: mol_type = other RNA
  modified_base 3: mod_base = OTHER; note = Uracil
  modified_base 10: mod_base = OTHER; note = Uracil
  modified_base 14: mod_base = OTHER; note = Uracil
  modified_base 15: mod_base = OTHER; note = Uracil
  modified_base 17: mod_base = OTHER; note = Uracil
gctcaagcatggattctaa

SEQ ID NO: 8 - RNA, 19 nt, Synthetic construct
  misc_feature 1..19: note = synthetic Polynucleotide
  source 1..19: mol_type = other RNA
  modified_base 4: mod_base = OTHER; note = Uracil
  modified_base 10: mod_base = OTHER; note = Uracil
  modified_base 13: mod_base = OTHER; note = Uracil
  modified_base 16: mod_base = OTHER; note = Uracil
  modified_base 18: mod_base = OTHER; note = Uracil
caatcaaggtagtaatatg

SEQ ID NO: 9 - RNA, 19 nt, Synthetic construct
  misc_feature 1..19: note = synthetic Polynucleotide
  source 1..19: mol_type = other RNA
  modified_base 5: mod_base = OTHER; note = Uracil
  modified_base 6: mod_base = OTHER; note = Uracil
  modified_base 10: mod_base = OTHER; note = Uracil
  modified_base 18: mod_base = OTHER; note = Uracil
gggcttcgctacaggaatc

SEQ ID NO: 10 - RNA, 19 nt, Synthetic construct
  misc_feature 1..19: note = synthetic Polynucleotide
  source 1..19: mol_type = other RNA
  modified_base 6: mod_base = OTHER; note = Uracil
  modified_base 10: mod_base = OTHER; note = Uracil
  modified_base 11: mod_base = OTHER; note = Uracil
  modified_base 12: mod_base = OTHER; note = Uracil
  modified_base 15: mod_base = OTHER; note = Uracil
  modified_base 18: mod_base = OTHER; note = Uracil
cagggtggatttggtaata

SEQ ID NO: 11 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 31: mod_base = OTHER; note = Uracil
  modified_base 35: mod_base = OTHER; note = Uracil
  modified_base 37: mod_base = OTHER; note = Uracil
  modified_base 39: mod_base = OTHER; note = Uracil
  modified_base 41: mod_base = OTHER; note = Uracil
  modified_base 42: mod_base = OTHER; note = Uracil
  misc_feature 24..43: note = spacer sequence
agaaatccgtctttcattgacggtgccccatcactctctctta

SEQ ID NO: 12 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 25: mod_base = OTHER; note = Uracil
  modified_base 26: mod_base = OTHER; note = Uracil
  modified_base 30: mod_base = OTHER; note = Uracil
  modified_base 31: mod_base = OTHER; note = Uracil
  modified_base 32: mod_base = OTHER; note = Uracil
  modified_base 33: mod_base = OTHER; note = Uracil
  modified_base 34: mod_base = OTHER; note = Uracil
  modified_base 39: mod_base = OTHER; note = Uracil
  modified_base 40: mod_base = OTHER; note = Uracil
  modified_base 42: mod_base = OTHER; note = Uracil
  misc_feature 24..43: note = spacer sequence
agaaatccgtctttcattgacggattggatttttaaaattata

SEQ ID NO: 13 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 26: mod_base = OTHER; note = Uracil
  modified_base 27: mod_base = OTHER; note = Uracil
  modified_base 28: mod_base = OTHER; note = Uracil
  modified_base 29: mod_base = OTHER; note = Uracil
  modified_base 30: mod_base = OTHER; note = Uracil
  modified_base 35: mod_base = OTHER; note = Uracil
  modified_base 36: mod_base = OTHER; note = Uracil
  modified_base 38: mod_base = OTHER; note = Uracil
  modified_base 40: mod_base = OTHER; note = Uracil
  modified_base 41: mod_base = OTHER; note = Uracil
  misc_feature 25..43: note = spacer sequence
agaaatccgtctttcattgacgggatttttaaaattatattca

SEQ ID NO: 14 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 25: mod_base = OTHER; note = Uracil
  modified_base 30: mod_base = OTHER; note = Uracil
  modified_base 31: mod_base = OTHER; note = Uracil
  modified_base 33: mod_base = OTHER; note = Uracil
  modified_base 35: mod_base = OTHER; note = Uracil
  modified_base 36: mod_base = OTHER; note = Uracil
  modified_base 39: mod_base = OTHER; note = Uracil
  modified_base 41: mod_base = OTHER; note = Uracil
  modified_base 42: mod_base = OTHER; note = Uracil
  misc_feature 24..43: note = spacer sequence
agaaatccgtctttcattgacggttaaaattatattcatattg

SEQ ID NO: 15 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 29: mod_base = OTHER; note = Uracil
  modified_base 30: mod_base = OTHER; note = Uracil
  modified_base 32: mod_base = OTHER; note = Uracil
  modified_base 34: mod_base = OTHER; note = Uracil
  modified_base 35: mod_base = OTHER; note = Uracil
  modified_base 38: mod_base = OTHER; note = Uracil
  modified_base 40: mod_base = OTHER; note = Uracil
  modified_base 41: mod_base = OTHER; note = Uracil
  misc_feature 24..43: note = spacer sequence
agaaatccgtctttcattgacggtaaaattatattcatattgc

SEQ ID NO: 16 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 28: mod_base = OTHER; note = Uracil
  modified_base 29: mod_base = OTHER; note = Uracil
  modified_base 31: mod_base = OTHER; note = Uracil
  modified_base 33: mod_base = OTHER; note = Uracil
  modified_base 34: mod_base = OTHER; note = Uracil
  modified_base 37: mod_base = OTHER; note = Uracil
  modified_base 39: mod_base = OTHER; note = Uracil
  modified_base 40: mod_base = OTHER; note = Uracil
  misc_feature 24..43: note = spacer sequence
agaaatccgtctttcattgacggaaaattatattcatattgca

SEQ ID NO: 17 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 27: mod_base = OTHER; note = Uracil
  modified_base 28: mod_base = OTHER; note = Uracil
  modified_base 30: mod_base = OTHER; note = Uracil
  modified_base 32: mod_base = OTHER; note = Uracil
  modified_base 33: mod_base = OTHER; note = Uracil
  modified_base 36: mod_base = OTHER; note = Uracil
  modified_base 38: mod_base = OTHER; note = Uracil
  modified_base 39: mod_base = OTHER; note = Uracil
  misc_feature 24..43: note = spacer sequence
agaaatccgtctttcattgacggaaattatattcatattgcag

SEQ ID NO: 18 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 26: mod_base = OTHER; note = Uracil
  modified_base 27: mod_base = OTHER; note = Uracil
  modified_base 30: mod_base = OTHER; note = Uracil
  modified_base 32: mod_base = OTHER; note = Uracil
  modified_base 33: mod_base = OTHER; note = Uracil
  modified_base 41: mod_base = OTHER; note = Uracil
  misc_feature 24..43: note = spacer sequence
agaaatccgtctttcattgacggtattcatattgcaggactcg

SEQ ID NO: 19 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 25: mod_base = OTHER; note = Uracil
  modified_base 27: mod_base = OTHER; note = Uracil
  modified_base 28: mod_base = OTHER; note = Uracil
  modified_base 36: mod_base = OTHER; note = Uracil
  misc_feature 24..43: note = spacer sequence
agaaatccgtctttcattgacggatattgcaggactcggcaga

SEQ ID NO: 20 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 30: mod_base = OTHER; note = Uracil
  modified_base 43: mod_base = OTHER; note = Uracil
  misc_feature 24..43: note = spacer sequence
agaaatccgtctttcattgacggcaggactcggcagaagacct

SEQ ID NO: 21 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 34: mod_base = OTHER; note = Uracil
  modified_base 41: mod_base = OTHER; note = Uracil
  misc_feature 24..43: note = spacer sequence
agaaatccgtctttcattgacgggagagaaaggtagaaaataa

SEQ ID NO: 22 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 27: mod_base = OTHER; note = Uracil
  modified_base 29: mod_base = OTHER; note = Uracil
  modified_base 31: mod_base = OTHER; note = Uracil
  modified_base 33: mod_base = OTHER; note = Uracil
  modified_base 35: mod_base = OTHER; note = Uracil
  modified_base 41: mod_base = OTHER; note = Uracil
  modified_base 43: mod_base = OTHER; note = Uracil
  misc_feature 24..43: note = spacer sequence
agaaatccgtctttcattgacggggctctctgtgtgagcatgt

SEQ ID NO: 23 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 25: mod_base = OTHER; note = Uracil
  modified_base 29: mod_base = OTHER; note = Uracil
  modified_base 35: mod_base = OTHER; note = Uracil
  modified_base 38: mod_base = OTHER; note = Uracil
  misc_feature 24..43: note = spacer sequence
agaaatccgtctttcattgacggatgaatagcaatactgaaga

SEQ ID NO: 24 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 26: mod_base = OTHER; note = Uracil
  modified_base 30: mod_base = OTHER; note = Uracil
  modified_base 33: mod_base = OTHER; note = Uracil
  modified_base 37: mod_base = OTHER; note = Uracil
  modified_base 39: mod_base = OTHER; note = Uracil
  misc_feature 24..43: note = spacer sequence
agaaatccgtctttcattgacggggtaaataataaatataaga

SEQ ID NO: 25 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 25: mod_base = OTHER; note = Uracil
  modified_base 34: mod_base = OTHER; note = Uracil
  modified_base 36: mod_base = OTHER; note = Uracil
  modified_base 38: mod_base = OTHER; note = Uracil
  modified_base 40: mod_base = OTHER; note = Uracil
  modified_base 42: mod_base = OTHER; note = Uracil
  misc_feature 24..43: note = spacer sequence
agaaatccgtctttcattgacggttaggcaggctgtctgtctc

SEQ ID NO: 26 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 25: mod_base = OTHER; note = Uracil
  modified_base 27: mod_base = OTHER; note = Uracil
  modified_base 28: mod_base = OTHER; note = Uracil
  modified_base 29: mod_base = OTHER; note = Uracil
  modified_base 30: mod_base = OTHER; note = Uracil
  modified_base 32: mod_base = OTHER; note = Uracil
  modified_base 36: mod_base = OTHER; note = Uracil
  modified_base 37: mod_base = OTHER; note = Uracil
  modified_base 38: mod_base = OTHER; note = Uracil
  modified_base 40: mod_base = OTHER; note = Uracil
  modified_base 42: mod_base = OTHER; note = Uracil
  misc_feature 24..43: note = spacer sequence
agaaatccgtctttcattgacggttattttctacctttctctc

SEQ ID NO: 27 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 25: mod_base = OTHER; note = Uracil
  modified_base 26: mod_base = OTHER; note = Uracil
  modified_base 27: mod_base = OTHER; note = Uracil
  modified_base 29: mod_base = OTHER; note = Uracil
  modified_base 33: mod_base = OTHER; note = Uracil
  modified_base 34: mod_base = OTHER; note = Uracil
  modified_base 35: mod_base = OTHER; note = Uracil
  modified_base 37: mod_base = OTHER; note = Uracil
  modified_base 39: mod_base = OTHER; note = Uracil
  misc_feature 24..43: note = spacer sequence
agaaatccgtctttcattgacggttttctacctttctctcgaa

SEQ ID NO: 28 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 26: mod_base = OTHER; note = Uracil
  modified_base 30: mod_base = OTHER; note = Uracil
  modified_base 31: mod_base = OTHER; note = Uracil
  modified_base 32: mod_base = OTHER; note = Uracil
  modified_base 34: mod_base = OTHER; note = Uracil
  modified_base 36: mod_base = OTHER; note = Uracil
  modified_base 43: mod_base = OTHER; note = Uracil
  misc_feature 24..43: note = spacer sequence
agaaatccgtctttcattgacggtctacctttctctcgaaggt

SEQ ID NO: 29 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 25: mod_base = OTHER; note = Uracil
  modified_base 29: mod_base = OTHER; note = Uracil
  modified_base 30: mod_base = OTHER; note = Uracil
  modified_base 31: mod_base = OTHER; note = Uracil
  modified_base 33: mod_base = OTHER; note = Uracil
  modified_base 35: mod_base = OTHER; note = Uracil
  modified_base 42: mod_base = OTHER; note = Uracil
  misc_feature 24..43: note = spacer sequence
agaaatccgtctttcattgacggctacctttctctcgaaggtc

SEQ ID NO: 30 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 28: mod_base = OTHER; note = Uracil
  modified_base 29: mod_base = OTHER; note = Uracil
  modified_base 30: mod_base = OTHER; note = Uracil
  modified_base 32: mod_base = OTHER; note = Uracil
  modified_base 34: mod_base = OTHER; note = Uracil
  modified_base 41: mod_base = OTHER; note = Uracil
  modified_base 43: mod_base = OTHER; note = Uracil
  misc_feature 24..43: note = spacer sequence
agaaatccgtctttcattgacggtacctttctctcgaaggtct

SEQ ID NO: 31 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 25: mod_base = OTHER; note = Uracil
  modified_base 27: mod_base = OTHER; note = Uracil
  modified_base 34: mod_base = OTHER; note = Uracil
  modified_base 36: mod_base = OTHER; note = Uracil
  modified_base 37: mod_base = OTHER; note = Uracil
  modified_base 39: mod_base = OTHER; note = Uracil
  misc_feature 24..43: note = spacer sequence
agaaatccgtctttcattgacggctctcgaaggtcttctgccg

SEQ ID NO: 32 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 26: mod_base = OTHER; note = Uracil
  modified_base 33: mod_base = OTHER; note = Uracil
  modified_base 35: mod_base = OTHER; note = Uracil
  modified_base 36: mod_base = OTHER; note = Uracil
  modified_base 38: mod_base = OTHER; note = Uracil
  misc_feature 24..43: note = spacer sequence
agaaatccgtctttcattgacggtctcgaaggtcttctgccga

SEQ ID NO: 33 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 31: mod_base = OTHER; note = Uracil
  modified_base 34: mod_base = OTHER; note = Uracil
  modified_base 39: mod_base = OTHER; note = Uracil
  modified_base 41: mod_base = OTHER; note = Uracil
  misc_feature 24..43: note = spacer sequence
agaaatccgtctttcattgacggtgccgagtcctgcaatatga

SEQ ID NO: 34 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 24: mod_base = OTHER; note = Uracil
  modified_base 30: mod_base = OTHER; note = Uracil
  modified_base 35: mod_base = OTHER; note = Uracil
  modified_base 36: mod_base = OTHER; note = Uracil
  misc_feature 24..43: note = spacer sequence
agaaatccgtctttcattgacggtaaaaatccaattaagagag

SEQ ID NO: 35 - RNA, 43 nt, Synthetic construct
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 26: mod_base = OTHER; note = Uracil
  modified_base 30: mod_base = OTHER; note = Uracil
  modified_base 37: mod_base = OTHER; note = Uracil
  misc_feature 24..43: note = spacer sequence
agaaatccgtctttcattgacggagtgcgtacgagctcggacg

SEQ ID NO: 36 - RNA, 23 nt, Synthetic construct
  misc_feature 1..23: note = synthetic Polynucleotide
  source 1..23: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
agaaatccgtctttcattgacgg

SEQ ID NO: 37 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 35: mod_base = OTHER; note = Uracil
  modified_base 40: mod_base = OTHER; note = Uracil
  modified_base 41: mod_base = OTHER; note = Uracil
ggtcccatcggaacgggttgtggttccgacaaaatccaattaagagagag

SEQ ID NO: 38 - RNA, 30 nt, Synthetic construct
  misc_feature 1..30: note = synthetic Polynucleotide
  source 1..30: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
ggtcccatcggaacgggttgtggttccgac

SEQ ID NO: 39 - RNA, 43 nt, Synthetic construct; identical to SEQ ID NO: 22
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 27: mod_base = OTHER; note = Uracil
  modified_base 29: mod_base = OTHER; note = Uracil
  modified_base 31: mod_base = OTHER; note = Uracil
  modified_base 33: mod_base = OTHER; note = Uracil
  modified_base 35: mod_base = OTHER; note = Uracil
  modified_base 41: mod_base = OTHER; note = Uracil
  modified_base 43: mod_base = OTHER; note = Uracil

SEQ ID NO: 40 - RNA, 43 nt, Synthetic construct; identical to SEQ ID NO: 31
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 25: mod_base = OTHER; note = Uracil
  modified_base 27: mod_base = OTHER; note = Uracil
  modified_base 34: mod_base = OTHER; note = Uracil
  modified_base 36: mod_base = OTHER; note = Uracil
  modified_base 37: mod_base = OTHER; note = Uracil
  modified_base 39: mod_base = OTHER; note = Uracil

SEQ ID NO: 41 - RNA, 43 nt, Synthetic construct; identical to SEQ ID NO: 35
  misc_feature 1..43: note = synthetic Polynucleotide
  source 1..43: mol_type = other RNA
  modified_base 6: mod_base = OTHER; note = Uracil
  modified_base 10: mod_base = OTHER; note = Uracil
  modified_base 12: mod_base = OTHER; note = Uracil
  modified_base 13: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 26: mod_base = OTHER; note = Uracil
  modified_base 30: mod_base = OTHER; note = Uracil
  modified_base 37: mod_base = OTHER; note = Uracil

SEQ ID NO: 42 - DNA, 20 bp, Synthetic construct
  misc_feature 1..20: note = synthetic Polynucleotide
  source 1..20: mol_type = other DNA
attggatttttaaaattata

SEQ ID NO: 43 - DNA, 20 bp, Synthetic construct
  misc_feature 1..20: note = synthetic Polynucleotide
  source 1..20: mol_type = other DNA
gatttttaaaattatattca

SEQ ID NO: 44 - DNA, 20 bp, Synthetic construct
  misc_feature 1..20: note = synthetic Polynucleotide
  source 1..20: mol_type = other DNA
aaattatattcatattgcag

SEQ ID NO: 45 - DNA, 20 bp, Synthetic construct
  misc_feature 1..20: note = synthetic Polynucleotide
  source 1..20: mol_type = other DNA
tattcatattgcaggactcg

SEQ ID NO: 46 - DNA, 20 bp, Synthetic construct
  misc_feature 1..20: note = synthetic Polynucleotide
  source 1..20: mol_type = other DNA
caggactcggcagaagacct

SEQ ID NO: 47 - DNA, 20 bp, Synthetic construct
  misc_feature 1..20: note = synthetic Polynucleotide
  source 1..20: mol_type = other DNA
ttttctacctttctctcgaa

SEQ ID NO: 48 - DNA, 20 bp, Synthetic construct
  misc_feature 1..20: note = synthetic Polynucleotide
  source 1..20: mol_type = other DNA
aaaatccaattaagagagag

SEQ ID NO: 49 - RNA, 56 nt, Synthetic construct
  misc_feature 1..56: note = synthetic Polynucleotide
  source 1..56: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
  modified_base 38: mod_base = OTHER; note = Uracil
  modified_base 39: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  modified_base 47: mod_base = OTHER; note = Uracil
  modified_base 52: mod_base = OTHER; note = Uracil
  modified_base 53: mod_base = OTHER; note = Uracil
  modified_base 55: mod_base = OTHER; note = Uracil
  misc_feature 37..56: note = spacer sequence
cttgttgtatatgtccttttataggtattaaacaacattggatttttaaaattata

SEQ ID NO: 50 - RNA, 56 nt, Synthetic construct
  misc_feature 1..56: note = synthetic Polynucleotide
  source 1..56: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
  modified_base 39: mod_base = OTHER; note = Uracil
  modified_base 40: mod_base = OTHER; note = Uracil
  modified_base 41: mod_base = OTHER; note = Uracil
  modified_base 42: mod_base = OTHER; note = Uracil
  modified_base 43: mod_base = OTHER; note = Uracil
  modified_base 48: mod_base = OTHER; note = Uracil
  modified_base 49: mod_base = OTHER; note = Uracil
  modified_base 51: mod_base = OTHER; note = Uracil
  modified_base 53: mod_base = OTHER; note = Uracil
  modified_base 54: mod_base = OTHER; note = Uracil
  misc_feature 37..56: note = spacer sequence
cttgttgtatatgtccttttataggtattaaacaacgatttttaaaattatattca

SEQ ID NO: 51 - RNA, 56 nt, Synthetic construct
  misc_feature 1..56: note = synthetic Polynucleotide
  source 1..56: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
  modified_base 40: mod_base = OTHER; note = Uracil
  modified_base 41: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  modified_base 49: mod_base = OTHER; note = Uracil
  modified_base 51: mod_base = OTHER; note = Uracil
  modified_base 52: mod_base = OTHER; note = Uracil
  misc_feature 37..56: note = spacer sequence
cttgttgtatatgtccttttataggtattaaacaacaaattatattcatattgcag

SEQ ID NO: 52 - RNA, 56 nt, Synthetic construct
  misc_feature 1..56: note = synthetic Polynucleotide
  source 1..56: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
  modified_base 37: mod_base = OTHER; note = Uracil
  modified_base 39: mod_base = OTHER; note = Uracil
  modified_base 40: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  modified_base 54: mod_base = OTHER; note = Uracil
  misc_feature 37..56: note = spacer sequence
cttgttgtatatgtccttttataggtattaaacaactattcatattgcaggactcg

SEQ ID NO: 53 - RNA, 56 nt, Synthetic construct
  misc_feature 1..56: note = synthetic Polynucleotide
  source 1..56: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
  modified_base 43: mod_base = OTHER; note = Uracil
  modified_base 56: mod_base = OTHER; note = Uracil
  misc_feature 37..56: note = spacer sequence
cttgttgtatatgtccttttataggtattaaacaaccaggactcggcagaagacct

SEQ ID NO: 54 - RNA, 56 nt, Synthetic construct
  misc_feature 1..56: note = synthetic Polynucleotide
  source 1..56: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
  modified_base 37: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 42: mod_base = OTHER; note = Uracil
  modified_base 46: mod_base = OTHER; note = Uracil
  modified_base 47: mod_base = OTHER; note = Uracil
  modified_base 48: mod_base = OTHER; note = Uracil
  modified_base 50: mod_base = OTHER; note = Uracil
  modified_base 52: mod_base = OTHER; note = Uracil
  misc_feature 37..56: note = spacer sequence
cttgttgtatatgtccttttataggtattaaacaacttttctacctttctctcgaa

SEQ ID NO: 55 - RNA, 56 nt, Synthetic construct
  misc_feature 1..56: note = synthetic Polynucleotide
  source 1..56: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
  modified_base 41: mod_base = OTHER; note = Uracil
  modified_base 46: mod_base = OTHER; note = Uracil
  modified_base 47: mod_base = OTHER; note = Uracil
  misc_feature 37..56: note = spacer sequence
cttgttgtatatgtccttttataggtattaaacaacaaaatccaattaagagagag

SEQ ID NO: 56 - RNA, 36 nt, Synthetic construct
  misc_feature 1..36: note = synthetic Polynucleotide
  source 1..36: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
cttgttgtatatgtccttttataggtattaaacaac

SEQ ID NO: 57 - DNA, 20 bp, Synthetic construct
  misc_feature 1..20: note = synthetic Polynucleotide
  source 1..20: mol_type = other DNA
attggatttttaaaattata

SEQ ID NO: 58 - DNA, 20 bp, Synthetic construct
  misc_feature 1..20: note = synthetic Polynucleotide
  source 1..20: mol_type = other DNA
aaattatattcatattgcag

SEQ ID NO: 59 - DNA, 20 bp, Synthetic construct
  misc_feature 1..20: note = synthetic Polynucleotide
  source 1..20: mol_type = other DNA
tattcatattgcaggactcg

SEQ ID NO: 60 - DNA, 20 bp, Synthetic construct
  misc_feature 1..20: note = synthetic Polynucleotide
  source 1..20: mol_type = other DNA
caggactcggcagaagacct

SEQ ID NO: 61 - DNA, 20 bp, Synthetic construct
  misc_feature 1..20: note = synthetic Polynucleotide
  source 1..20: mol_type = other DNA
ttttctacctttctctcgaa

SEQ ID NO: 62 - DNA, 20 bp, Synthetic construct
  misc_feature 1..20: note = synthetic Polynucleotide
  source 1..20: mol_type = other DNA
aaaatccaattaagagagag

SEQ ID NO: 63 - DNA, 20 bp, Synthetic construct
  misc_feature 1..20: note = synthetic Polynucleotide
  source 1..20: mol_type = other DNA
ccccatcactctctcttaat

SEQ ID NO: 64 - DNA, 20 bp, Synthetic construct
  misc_feature 1..20: note = synthetic Polynucleotide
  source 1..20: mol_type = other DNA
tcactctctcttaattggat

SEQ ID NO: 65 - DNA, 20 bp, Synthetic construct
  misc_feature 1..20: note = synthetic Polynucleotide
  source 1..20: mol_type = other DNA
ctctctcttaattggatttt

SEQ ID NO: 66 - DNA, 20 bp, Synthetic construct
  misc_feature 1..20: note = synthetic Polynucleotide
  source 1..20: mol_type = other DNA
attggatttttaaaattata

SEQ ID NO: 67 - DNA, 20 bp, Synthetic construct
  misc_feature 1..20: note = synthetic Polynucleotide
  source 1..20: mol_type = other DNA
ttggatttttaaaattatat

SEQ ID NO: 68 - DNA, 20 bp, Synthetic construct
  misc_feature 1..20: note = synthetic Polynucleotide
  source 1..20: mol_type = other DNA
tttttaaaattatattcata

SEQ ID NO: 69 - DNA, 20 bp, Synthetic construct
  misc_feature 1..20: note = synthetic Polynucleotide
  source 1..20: mol_type = other DNA
aaattatattcatattgcag

SEQ ID NO: 70 - DNA, 20 bp, Synthetic construct
  misc_feature 1..20: note = synthetic Polynucleotide
  source 1..20: mol_type = other DNA
aattatattcatattgcagg

SEQ ID NO: 71 - DNA, 20 bp, Synthetic construct
  misc_feature 1..20: note = synthetic Polynucleotide
  source 1..20: mol_type = other DNA
attatattcatattgcagga

SEQ ID NO: 72 - DNA, 20 bp, Synthetic construct
  misc_feature 1..20: note = synthetic Polynucleotide
  source 1..20: mol_type = other DNA
ttatattcatattgcaggac

SEQ ID NO: 73 - DNA, 20 bp, Synthetic construct
  misc_feature 1..20: note = synthetic Polynucleotide
  source 1..20: mol_type = other DNA
tattcatattgcaggactcg

SEQ ID NO: 74 - DNA, 20 bp, Synthetic construct
  misc_feature 1..20: note = synthetic Polynucleotide
  source 1..20: mol_type = other DNA
ttcatattgcaggactcggc

SEQ ID NO: 75 - DNA, 20 bp, Synthetic construct
  misc_feature 1..20: note = synthetic Polynucleotide
  source 1..20: mol_type = other DNA
tattgcaggactcggcagaa

SEQ ID NO: 76 - DNA, 20 bp, Synthetic construct
  misc_feature 1..20: note = synthetic Polynucleotide
  source 1..20: mol_type = other DNA
ttgcaggactcggcagaaga

SEQ ID NO: 77 - DNA, 20 bp, Synthetic construct
  misc_feature 1..20: note = synthetic Polynucleotide
  source 1..20: mol_type = other DNA
caggactcggcagaagacct

SEQ ID NO: 78 - DNA, 20 bp, Synthetic construct
  misc_feature 1..20: note = synthetic Polynucleotide
  source 1..20: mol_type = other DNA
ggactcggcagaagaccttc

SEQ ID NO: 79 - DNA, 20 bp, Synthetic construct
  misc_feature 1..20: note = synthetic Polynucleotide
  source 1..20: mol_type = other DNA
gactcggcagaagaccttcg

SEQ ID NO: 80 - DNA, 20 bp, Synthetic construct
  misc_feature 1..20: note = synthetic Polynucleotide
  source 1..20: mol_type = other DNA
actcggcagaagaccttcga

SEQ ID NO: 81 - DNA, 20 bp, Synthetic construct
  misc_feature 1..20: note = synthetic Polynucleotide
  source 1..20: mol_type = other DNA
ctcggcagaagaccttcgag

SEQ ID NO: 82 - DNA, 20 bp, Synthetic construct
  misc_feature 1..20: note = synthetic Polynucleotide
  source 1..20: mol_type = other DNA
gcagaagaccttcgagagaa

SEQ ID NO: 83 - DNA, 20 bp, Synthetic construct
  misc_feature 1..20: note = synthetic Polynucleotide
  source 1..20: mol_type = other DNA
cagaagaccttcgagagaaa

SEQ ID NO: 84 - DNA, 20 bp, Synthetic construct
  misc_feature 1..20: note = synthetic Polynucleotide
  source 1..20: mol_type = other DNA
gaagaccttcgagagaaagg

SEQ ID NO: 85 - DNA, 20 bp, Synthetic construct
  misc_feature 1..20: note = synthetic Polynucleotide
  source 1..20: mol_type = other DNA
aagaccttcgagagaaaggt

SEQ ID NO: 86 - DNA, 20 bp, Synthetic construct
  misc_feature 1..20: note = synthetic Polynucleotide
  source 1..20: mol_type = other DNA
agaccttcgagagaaaggta

SEQ ID NO: 87 - DNA, 20 bp, Synthetic construct
  misc_feature 1..20: note = synthetic Polynucleotide
  source 1..20: mol_type = other DNA
gaccttcgagagaaaggtag

SEQ ID NO: 88 - DNA, 20 bp, Synthetic construct
  misc_feature 1..20: note = synthetic Polynucleotide
  source 1..20: mol_type = other DNA
accttcgagagaaaggtaga

SEQ ID NO: 89 - DNA, 20 bp, Synthetic construct
  misc_feature 1..20: note = synthetic Polynucleotide
  source 1..20: mol_type = other DNA
ccttcgagagaaaggtagaa

SEQ ID NO: 90 - DNA, 20 bp, Synthetic construct
  misc_feature 1..20: note = synthetic Polynucleotide
  source 1..20: mol_type = other DNA
agagaaaggtagaaaataag

SEQ ID NO: 91 - DNA, 20 bp, Synthetic construct
  misc_feature 1..20: note = synthetic Polynucleotide
  source 1..20: mol_type = other DNA
gagccaaattcttattttct

SEQ ID NO: 92 - DNA, 20 bp, Synthetic construct
  misc_feature 1..20: note = synthetic Polynucleotide
  source 1..20: mol_type = other DNA
agccaaattcttattttcta

SEQ ID NO: 93 - DNA, 20 bp, Synthetic construct
  misc_feature 1..20: note = synthetic Polynucleotide
  source 1..20: mol_type = other DNA
gccaaattcttattttctac

SEQ ID NO: 94 - DNA, 20 bp, Synthetic construct
  misc_feature 1..20: note = synthetic Polynucleotide
  source 1..20: mol_type = other DNA
ccaaattcttattttctacc

SEQ ID NO: 95 - DNA, 20 bp, Synthetic construct
  misc_feature 1..20: note = synthetic Polynucleotide
  source 1..20: mol_type = other DNA
aattcttattttctaccttt

SEQ ID NO: 96 - DNA, 20 bp, Synthetic construct
  misc_feature 1..20: note = synthetic Polynucleotide
  source 1..20: mol_type = other DNA
attcttattttctacctttc

SEQ ID NO: 97 - DNA, 20 bp, Synthetic construct
  misc_feature 1..20: note = synthetic Polynucleotide
  source 1..20: mol_type = other DNA
ttcttattttctacctttct

SEQ ID NO: 98 - DNA, 20 bp, Synthetic construct
  misc_feature 1..20: note = synthetic Polynucleotide
  source 1..20: mol_type = other DNA
ttttctacctttctctcgaa

SEQ ID NO: 99 - DNA, 20 bp, Synthetic construct
  misc_feature 1..20: note = synthetic Polynucleotide
  source 1..20: mol_type = other DNA
cctttctctcgaaggtcttc

SEQ ID NO: 100 - DNA, 20 bp, Synthetic construct
  misc_feature 1..20: note = synthetic Polynucleotide
  source 1..20: mol_type = other DNA
aaggtcttctgccgagtcct

SEQ ID NO: 101 - DNA, 20 bp, Synthetic construct
  misc_feature 1..20: note = synthetic Polynucleotide
  source 1..20: mol_type = other DNA
aggtcttctgccgagtcctg

SEQ ID NO: 102 - DNA, 20 bp, Synthetic construct
  misc_feature 1..20: note = synthetic Polynucleotide
  source 1..20: mol_type = other DNA
ggtcttctgccgagtcctgc

SEQ ID NO: 103 - DNA, 20 bp, Synthetic construct
  misc_feature 1..20: note = synthetic Polynucleotide
  source 1..20: mol_type = other DNA
gtcttctgccgagtcctgca

SEQ ID NO: 104 - DNA, 20 bp, Synthetic construct
  misc_feature 1..20: note = synthetic Polynucleotide
  source 1..20: mol_type = other DNA
tcttctgccgagtcctgcaa

SEQ ID NO: 105 - DNA, 20 bp, Synthetic construct
  misc_feature 1..20: note = synthetic Polynucleotide
  source 1..20: mol_type = other DNA
ccgagtcctgcaatatgaat

SEQ ID NO: 106 - DNA, 20 bp, Synthetic construct
  misc_feature 1..20: note = synthetic Polynucleotide
  source 1..20: mol_type = other DNA
agtcctgcaatatgaatata

SEQ ID NO: 107 - DNA, 20 bp, Synthetic construct
  misc_feature 1..20: note = synthetic Polynucleotide
  source 1..20: mol_type = other DNA
gtcctgcaatatgaatataa

SEQ ID NO: 108 - DNA, 20 bp, Synthetic construct
  misc_feature 1..20: note = synthetic Polynucleotide
  source 1..20: mol_type = other DNA
tcctgcaatatgaatataat

SEQ ID NO: 109 - DNA, 20 bp, Synthetic construct
  misc_feature 1..20: note = synthetic Polynucleotide
  source 1..20: mol_type = other DNA
tatgaatataattttaaaaa

SEQ ID NO: 110 - DNA, 20 bp, Synthetic construct
  misc_feature 1..20: note = synthetic Polynucleotide
  source 1..20: mol_type = other DNA
atataattttaaaaatccaa

SEQ ID NO: 111 - DNA, 20 bp, Synthetic construct
  misc_feature 1..20: note = synthetic Polynucleotide
  source 1..20: mol_type = other DNA
tataattttaaaaatccaat

SEQ ID NO: 112 - DNA, 20 bp, Synthetic construct
  misc_feature 1..20: note = synthetic Polynucleotide
  source 1..20: mol_type = other DNA
taattttaaaaatccaatta

SEQ ID NO: 113 - DNA, 20 bp, Synthetic construct
  misc_feature 1..20: note = synthetic Polynucleotide
  source 1..20: mol_type = other DNA
attttaaaaatccaattaag

SEQ ID NO: 114 - DNA, 20 bp, Synthetic construct
  misc_feature 1..20: note = synthetic Polynucleotide
  source 1..20: mol_type = other DNA
ttttaaaaatccaattaaga

SEQ ID NO: 115 - DNA, 20 bp, Synthetic construct
  misc_feature 1..20: note = synthetic Polynucleotide
  source 1..20: mol_type = other DNA
aaaatccaattaagagagag

SEQ ID NO: 116 - DNA, 20 bp, Synthetic construct
  misc_feature 1..20: note = synthetic Polynucleotide
  source 1..20: mol_type = other DNA
aaatccaattaagagagagt

SEQ ID NO: 117 - DNA, 20 bp, Synthetic construct
  misc_feature 1..20: note = synthetic Polynucleotide
  source 1..20: mol_type = other DNA
aatccaattaagagagagtg

SEQ ID NO: 118 - DNA, 20 bp, Synthetic construct
  misc_feature 1..20: note = synthetic Polynucleotide
  source 1..20: mol_type = other DNA
atccaattaagagagagtga

SEQ ID NO: 119 - DNA, 20 bp, Synthetic construct
  misc_feature 1..20: note = synthetic Polynucleotide
  source 1..20: mol_type = other DNA
tccaattaagagagagtgat

SEQ ID NO: 120 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 39: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  modified_base 47: mod_base = OTHER; note = Uracil
  modified_base 52: mod_base = OTHER; note = Uracil
  modified_base 53: mod_base = OTHER; note = Uracil
  modified_base 55: mod_base = OTHER; note = Uracil
  misc_feature 37..56: note = spacer sequence
cctgttgtgaatactcttttataggtatcaaacaacattggatttttaaaattata

SEQ ID NO: 121 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 40: mod_base = OTHER; note = Uracil
  modified_base 41: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  modified_base 49: mod_base = OTHER; note = Uracil
  modified_base 51: mod_base = OTHER; note = Uracil
  modified_base 52: mod_base = OTHER; note = Uracil
  misc_feature 37..56: note = spacer sequence
cctgttgtgaatactcttttataggtatcaaacaacaaattatattcatattgcag

SEQ ID NO: 122 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 39: mod_base = OTHER; note = Uracil
  modified_base 40: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  modified_base 54: mod_base = OTHER; note = Uracil
  misc_feature 37..56: note = spacer sequence
cctgttgtgaatactcttttataggtatcaaacaactattcatattgcaggactcg

SEQ ID NO: 123 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 43: mod_base = OTHER; note = Uracil
  modified_base 56: mod_base = OTHER; note = Uracil
  misc_feature 37..56: note = spacer sequence
cctgttgtgaatactcttttataggtatcaaacaaccaggactcggcagaagacct

SEQ ID NO: 124 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 42: mod_base = OTHER; note = Uracil
  modified_base 46: mod_base = OTHER; note = Uracil
  modified_base 47: mod_base = OTHER; note = Uracil
  modified_base 48: mod_base = OTHER; note = Uracil
  modified_base 50: mod_base = OTHER; note = Uracil
  modified_base 52: mod_base = OTHER; note = Uracil
  misc_feature 37..56: note = spacer sequence
cctgttgtgaatactcttttataggtatcaaacaacttttctacctttctctcgaa

SEQ ID NO: 125 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 41: mod_base = OTHER; note = Uracil
  modified_base 46: mod_base = OTHER; note = Uracil
  modified_base 47: mod_base = OTHER; note = Uracil
  misc_feature 37..56: note = spacer sequence
cctgttgtgaatactcttttataggtatcaaacaacaaaatccaattaagagagag

SEQ ID NO: 126 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 42: mod_base = OTHER; note = Uracil
  modified_base 46: mod_base = OTHER; note = Uracil
  modified_base 48: mod_base = OTHER; note = Uracil
  modified_base 50: mod_base = OTHER; note = Uracil
  modified_base 52: mod_base = OTHER; note = Uracil
  modified_base 53: mod_base = OTHER; note = Uracil
  modified_base 56: mod_base = OTHER; note = Uracil
  misc_feature 36..56: note = spacer sequence
cctgttgtgaatactcttttataggtatcaaacaacccccatcactctctcttaat

SEQ ID NO: 127 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 41: mod_base = OTHER; note = Uracil
  modified_base 43: mod_base = OTHER; note = Uracil
  modified_base 45: mod_base = OTHER; note = Uracil
  modified_base 47: mod_base = OTHER; note = Uracil
  modified_base 48: mod_base = OTHER; note = Uracil
  modified_base 51: mod_base = OTHER; note = Uracil
  modified_base 52: mod_base = OTHER; note = Uracil
  modified_base 56: mod_base = OTHER; note = Uracil
  misc_feature 37..56: note = spacer sequence
cctgttgtgaatactcttttataggtatcaaacaactcactctctcttaattggat

SEQ ID NO: 128 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 40: mod_base = OTHER; note = Uracil
  modified_base 42: mod_base = OTHER; note = Uracil
  modified_base 44: mod_base = OTHER; note = Uracil
  modified_base 45: mod_base = OTHER; note = Uracil
  modified_base 48: mod_base = OTHER; note = Uracil
  modified_base 49: mod_base = OTHER; note = Uracil
  modified_base 53: mod_base = OTHER; note = Uracil
  modified_base 54: mod_base = OTHER; note = Uracil
  modified_base 55: mod_base = OTHER; note = Uracil
  modified_base 56: mod_base = OTHER; note = Uracil
  misc_feature 37..56: note = spacer sequence
cctgttgtgaatactcttttataggtatcaaacaacctctctcttaattggatttt

SEQ ID NO: 129 - RNA, 56 nt, Synthetic construct; identical to SEQ ID NO: 120
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 39: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  modified_base 47: mod_base = OTHER; note = Uracil
  modified_base 52: mod_base = OTHER; note = Uracil
  modified_base 53: mod_base = OTHER; note = Uracil
  modified_base 55: mod_base = OTHER; note = Uracil
  misc_feature 37..56: note = spacer sequence

SEQ ID NO: 130 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 42: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  modified_base 51: mod_base = OTHER; note = Uracil
  modified_base 52: mod_base = OTHER; note = Uracil
  modified_base 54: mod_base = OTHER; note = Uracil
  modified_base 56: mod_base = OTHER; note = Uracil
  misc_feature 37..56: note = spacer sequence
cctgttgtgaatactcttttataggtatcaaacaacttggatttttaaaattatat

SEQ ID NO: 131 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 41: mod_base = OTHER; note = Uracil
  modified_base 46: mod_base = OTHER; note = Uracil
  modified_base 47: mod_base = OTHER; note = Uracil
  modified_base 49: mod_base = OTHER; note = Uracil
  modified_base 51: mod_base = OTHER; note = Uracil
  modified_base 52: mod_base = OTHER; note = Uracil
  modified_base 55: mod_base = OTHER; note = Uracil
  misc_feature 37..56: note = spacer sequence
cctgttgtgaatactcttttataggtatcaaacaactttttaaaattatattcata

SEQ ID NO: 132 - RNA, 56 nt, Synthetic construct; identical to SEQ ID NO: 121
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 40: mod_base = OTHER; note = Uracil
  modified_base 41: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  modified_base 49: mod_base = OTHER; note = Uracil
  modified_base 51: mod_base = OTHER; note = Uracil
  modified_base 52: mod_base = OTHER; note = Uracil
  misc_feature 37..56: note = spacer sequence

SEQ ID NO: 133 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 39: mod_base = OTHER; note = Uracil
  modified_base 40: mod_base = OTHER; note = Uracil
  modified_base 42: mod_base = OTHER; note = Uracil
  modified_base 44: mod_base = OTHER; note = Uracil
  modified_base 45: mod_base = OTHER; note = Uracil
  modified_base 48: mod_base = OTHER; note = Uracil
  modified_base 50: mod_base = OTHER; note = Uracil
  modified_base 51: mod_base = OTHER; note = Uracil
  misc_feature 37..56: note = spacer sequence
cctgttgtgaatactcttttataggtatcaaacaacaattatattcatattgcagg

SEQ ID NO: 134 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 39: mod_base = OTHER; note = Uracil
  modified_base 41: mod_base = OTHER; note = Uracil
  modified_base 43: mod_base = OTHER; note = Uracil
  modified_base 44: mod_base = OTHER; note = Uracil
  modified_base 47: mod_base = OTHER; note = Uracil
  modified_base 49: mod_base = OTHER; note = Uracil
  modified_base 50: mod_base = OTHER; note = Uracil
  misc_feature 37..56: note = spacer sequence
cctgttgtgaatactcttttataggtatcaaacaacattatattcatattgcagga

SEQ ID NO: 135 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 40: mod_base = OTHER; note = Uracil
  modified_base 42: mod_base = OTHER; note = Uracil
  modified_base 43: mod_base = OTHER; note = Uracil
  modified_base 46: mod_base = OTHER; note = Uracil
  modified_base 48: mod_base = OTHER; note = Uracil
  modified_base 49: mod_base = OTHER; note = Uracil
  misc_feature 37..56: note = spacer sequence
cctgttgtgaatactcttttataggtatcaaacaacttatattcatattgcaggac

SEQ ID NO: 136 - RNA, 56 nt, Synthetic construct; identical to SEQ ID NO: 122
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 39: mod_base = OTHER; note = Uracil
  modified_base 40: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  modified_base 54: mod_base = OTHER; note = Uracil
  misc_feature 37..56: note = spacer sequence

SEQ ID NO: 137 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 41: mod_base = OTHER; note = Uracil
  modified_base 43: mod_base = OTHER; note = Uracil
  modified_base 44: mod_base = OTHER; note = Uracil
  modified_base 52: mod_base = OTHER; note = Uracil
  misc_feature 37..56: note = spacer sequence
cctgttgtgaatactcttttataggtatcaaacaacttcatattgcaggactcggc

SEQ ID NO: 138 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 39: mod_base = OTHER; note = Uracil
  modified_base 40: mod_base = OTHER; note = Uracil
  modified_base 48: mod_base = OTHER; note = Uracil
  misc_feature 37..56: note = spacer sequence
cctgttgtgaatactcttttataggtatcaaacaactattgcaggactcggcagaa

SEQ ID NO: 139 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 46: mod_base = OTHER; note = Uracil
  misc_feature 37..56: note = spacer sequence
cctgttgtgaatactcttttataggtatcaaacaacttgcaggactcggcagaaga

SEQ ID NO: 140 - RNA, 56 nt, Synthetic construct; identical to SEQ ID NO: 123
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 43: mod_base = OTHER; note = Uracil
  modified_base 56: mod_base = OTHER; note = Uracil
  misc_feature 37..56: note = spacer sequence

SEQ ID NO: 141 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 41: mod_base = OTHER; note = Uracil
  modified_base 54: mod_base = OTHER; note = Uracil
  modified_base 55: mod_base = OTHER; note = Uracil
  misc_feature 37..56: note = spacer sequence
cctgttgtgaatactcttttataggtatcaaacaacggactcggcagaagaccttc

SEQ ID NO: 142 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 40: mod_base = OTHER; note = Uracil
  modified_base 53: mod_base = OTHER; note = Uracil
  modified_base 54: mod_base = OTHER; note = Uracil
  misc_feature 37..56: note = spacer sequence
cctgttgtgaatactcttttataggtatcaaacaacgactcggcagaagaccttcg

SEQ ID NO: 143 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 39: mod_base = OTHER; note = Uracil
  modified_base 52: mod_base = OTHER; note = Uracil
  modified_base 53: mod_base = OTHER; note = Uracil
  misc_feature 37..56: note = spacer sequence
cctgttgtgaatactcttttataggtatcaaacaacactcggcagaagaccttcga

SEQ ID NO: 144 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 51: mod_base = OTHER; note = Uracil
  modified_base 52: mod_base = OTHER; note = Uracil
  misc_feature 37..56: note = spacer sequence
cctgttgtgaatactcttttataggtatcaaacaacctcggcagaagaccttcgag

SEQ ID NO: 145 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 47: mod_base = OTHER; note = Uracil
  modified_base 48: mod_base = OTHER; note = Uracil
  misc_feature 37..56: note = spacer sequence
cctgttgtgaatactcttttataggtatcaaacaacgcagaagaccttcgagagaa

SEQ ID NO: 146 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 46: mod_base = OTHER; note = Uracil
  modified_base 47: mod_base = OTHER; note = Uracil
  misc_feature 37..56: note = spacer sequence
cctgttgtgaatactcttttataggtatcaaacaaccagaagaccttcgagagaaa

SEQ ID NO: 147 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 44: mod_base = OTHER; note = Uracil
  modified_base 45: mod_base = OTHER; note = Uracil
  misc_feature 37..56: note = spacer sequence
cctgttgtgaatactcttttataggtatcaaacaacgaagaccttcgagagaaagg

SEQ ID NO: 148 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 43: mod_base = OTHER; note = Uracil
  modified_base 44: mod_base = OTHER; note = Uracil
  modified_base 56: mod_base = OTHER; note = Uracil
  misc_feature 37..56: note = spacer sequence
cctgttgtgaatactcttttataggtatcaaacaacaagaccttcgagagaaaggt

SEQ ID NO: 149 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 42: mod_base = OTHER; note = Uracil
  modified_base 43: mod_base = OTHER; note = Uracil
  modified_base 55: mod_base = OTHER; note = Uracil
  misc_feature 37..56: note = spacer sequence
cctgttgtgaatactcttttataggtatcaaacaacagaccttcgagagaaaggta

SEQ ID NO: 150 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 41: mod_base = OTHER; note = Uracil
  modified_base 42: mod_base = OTHER; note = Uracil
  modified_base 54: mod_base = OTHER; note = Uracil
  misc_feature 37..56: note = spacer sequence
cctgttgtgaatactcttttataggtatcaaacaacgaccttcgagagaaaggtag

SEQ ID NO: 151 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 40: mod_base = OTHER; note = Uracil
  modified_base 41: mod_base = OTHER; note = Uracil
  modified_base 53: mod_base = OTHER; note = Uracil
  misc_feature 37..56: note = spacer sequence
cctgttgtgaatactcttttataggtatcaaacaacaccttcgagagaaaggtaga

SEQ ID NO: 152 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 39: mod_base = OTHER; note = Uracil
  modified_base 40: mod_base = OTHER; note = Uracil
  modified_base 52: mod_base = OTHER; note = Uracil
  misc_feature 37..56: note = spacer sequence
cctgttgtgaatactcttttataggtatcaaacaacccttcgagagaaaggtagaa

SEQ ID NO: 153 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 46: mod_base = OTHER; note = Uracil
  modified_base 53: mod_base = OTHER; note = Uracil
  misc_feature 37..56: note = spacer sequence
cctgttgtgaatactcttttataggtatcaaacaacagagaaaggtagaaaataag

SEQ ID NO: 154 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 45: mod_base = OTHER; note = Uracil
  modified_base 46: mod_base = OTHER; note = Uracil
  modified_base 48: mod_base = OTHER; note = Uracil
  modified_base 49: mod_base = OTHER; note = Uracil
  modified_base 51: mod_base = OTHER; note = Uracil
  modified_base 52: mod_base = OTHER; note = Uracil
  modified_base 53: mod_base = OTHER; note = Uracil
  modified_base 54: mod_base = OTHER; note = Uracil
  modified_base 56: mod_base = OTHER; note = Uracil
  misc_feature 37..56: note = spacer sequence
cctgttgtgaatactcttttataggtatcaaacaacgagccaaattcttattttct

SEQ ID NO: 155 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 44: mod_base = OTHER; note = Uracil
  modified_base 45: mod_base = OTHER; note = Uracil
  modified_base 47: mod_base = OTHER; note = Uracil
  modified_base 48: mod_base = OTHER; note = Uracil
  modified_base 50: mod_base = OTHER; note = Uracil
  modified_base 51: mod_base = OTHER; note = Uracil
  modified_base 52: mod_base = OTHER; note = Uracil
  modified_base 53: mod_base = OTHER; note = Uracil
  modified_base 55: mod_base = OTHER; note = Uracil
  misc_feature 37..56: note = spacer sequence
cctgttgtgaatactcttttataggtatcaaacaacagccaaattcttattttcta

SEQ ID NO: 156 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 43: mod_base = OTHER; note = Uracil
  modified_base 44: mod_base = OTHER; note = Uracil
  modified_base 46: mod_base = OTHER; note = Uracil
  modified_base 47: mod_base = OTHER; note = Uracil
  modified_base 49: mod_base = OTHER; note = Uracil
  modified_base 50: mod_base = OTHER; note = Uracil
  modified_base 51: mod_base = OTHER; note = Uracil
  modified_base 52: mod_base = OTHER; note = Uracil
  modified_base 54: mod_base = OTHER; note = Uracil
  misc_feature 37..56: note = spacer sequence
cctgttgtgaatactcttttataggtatcaaacaacgccaaattcttattttctac

SEQ ID NO: 157 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 42: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  modified_base 48: mod_base = OTHER; note = Uracil
  modified_base 49: mod_base = OTHER; note = Uracil
  modified_base 50: mod_base = OTHER; note = Uracil
  modified_base 51: mod_base = OTHER; note = Uracil
  modified_base 53: mod_base = OTHER; note = Uracil
  misc_feature 37..56: note = spacer sequence
cctgttgtgaatactcttttataggtatcaaacaacccaaattcttattttctacc

SEQ ID NO: 158 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 39: mod_base = OTHER; note = Uracil
  modified_base 40: mod_base = OTHER; note = Uracil
  modified_base 42: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  modified_base 47: mod_base = OTHER; note = Uracil
  modified_base 48: mod_base = OTHER; note = Uracil
  modified_base 50: mod_base = OTHER; note = Uracil
  modified_base 54: mod_base = OTHER; note = Uracil
  modified_base 55: mod_base = OTHER; note = Uracil
  modified_base 56: mod_base = OTHER; note = Uracil
  misc_feature 37..56: note = spacer sequence
cctgttgtgaatactcttttataggtatcaaacaacaattcttattttctaccttt

SEQ ID NO: 159 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 39: mod_base = OTHER; note = Uracil
  modified_base 41: mod_base = OTHER; note = Uracil
  modified_base 42: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  modified_base 47: mod_base = OTHER; note = Uracil
  modified_base 49: mod_base = OTHER; note = Uracil
  modified_base 53: mod_base = OTHER; note = Uracil
  modified_base 54: mod_base = OTHER; note = Uracil
  modified_base 55: mod_base = OTHER; note = Uracil
  misc_feature 37..56: note = spacer sequence
cctgttgtgaatactcttttataggtatcaaacaacattcttattttctacctttc

SEQ ID NO: 160 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 40: mod_base = OTHER; note = Uracil
  modified_base 41: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  modified_base 48: mod_base = OTHER; note = Uracil
  modified_base 52: mod_base = OTHER; note = Uracil
  modified_base 53: mod_base = OTHER; note = Uracil
  modified_base 54: mod_base = OTHER; note = Uracil
  modified_base 56: mod_base = OTHER; note = Uracil
  misc_feature 37..56: note = spacer sequence
cctgttgtgaatactcttttataggtatcaaacaacttcttattttctacctttct

SEQ ID NO: 161 - RNA, 56 nt, Synthetic construct; identical to SEQ ID NO: 124
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 42: mod_base = OTHER; note = Uracil
  modified_base 46: mod_base = OTHER; note = Uracil
  modified_base 47: mod_base = OTHER; note = Uracil
  modified_base 48: mod_base = OTHER; note = Uracil
  modified_base 50: mod_base = OTHER; note = Uracil
  modified_base 52: mod_base = OTHER; note = Uracil
  misc_feature 37..56: note = spacer sequence

SEQ ID NO: 162 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 39: mod_base = OTHER; note = Uracil
  modified_base 40: mod_base = OTHER; note = Uracil
  modified_base 41: mod_base = OTHER; note = Uracil
  modified_base 43: mod_base = OTHER; note = Uracil
  modified_base 45: mod_base = OTHER; note = Uracil
  modified_base 52: mod_base = OTHER; note = Uracil
  modified_base 54: mod_base = OTHER; note = Uracil
  modified_base 55: mod_base = OTHER; note = Uracil
  misc_feature 36..56: note = spacer sequence
cctgttgtgaatactcttttataggtatcaaacaaccctttctctcgaaggtcttc

SEQ ID NO: 163 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 41: mod_base = OTHER; note = Uracil
  modified_base 43: mod_base = OTHER; note = Uracil
  modified_base 44: mod_base = OTHER; note = Uracil
  modified_base 46: mod_base = OTHER; note = Uracil
  modified_base 53: mod_base = OTHER; note = Uracil
  modified_base 56: mod_base = OTHER; note = Uracil
  misc_feature 37..56: note = spacer sequence
cctgttgtgaatactcttttataggtatcaaacaacaaggtcttctgccgagtcct

SEQ ID NO: 164 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 40: mod_base = OTHER; note = Uracil
  modified_base 42: mod_base = OTHER; note = Uracil
  modified_base 43: mod_base = OTHER; note = Uracil
  modified_base 45: mod_base = OTHER; note = Uracil
  modified_base 52: mod_base = OTHER; note = Uracil
  modified_base 55: mod_base = OTHER; note = Uracil
  misc_feature 37..56: note = spacer sequence
cctgttgtgaatactcttttataggtatcaaacaacaggtcttctgccgagtcctg

SEQ ID NO: 165 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 39: mod_base = OTHER; note = Uracil
  modified_base 41: mod_base = OTHER; note = Uracil
  modified_base 42: mod_base = OTHER; note = Uracil
  modified_base 44: mod_base = OTHER; note = Uracil
  modified_base 51: mod_base = OTHER; note = Uracil
  modified_base 54: mod_base = OTHER; note = Uracil
  misc_feature 37..56: note = spacer sequence
cctgttgtgaatactcttttataggtatcaaacaacggtcttctgccgagtcctgc

SEQ ID NO: 166 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 40: mod_base = OTHER; note = Uracil
  modified_base 41: mod_base = OTHER; note = Uracil
  modified_base 43: mod_base = OTHER; note = Uracil
  modified_base 50: mod_base = OTHER; note = Uracil
  modified_base 53: mod_base = OTHER; note = Uracil
  misc_feature 37..56: note = spacer sequence
cctgttgtgaatactcttttataggtatcaaacaacgtcttctgccgagtcctgca

SEQ ID NO: 167 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 39: mod_base = OTHER; note = Uracil
  modified_base 40: mod_base = OTHER; note = Uracil
  modified_base 42: mod_base = OTHER; note = Uracil
  modified_base 49: mod_base = OTHER; note = Uracil
  modified_base 52: mod_base = OTHER; note = Uracil
  misc_feature 37..56: note = spacer sequence
cctgttgtgaatactcttttataggtatcaaacaactcttctgccgagtcctgcaa

SEQ ID NO: 168 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 42: mod_base = OTHER; note = Uracil
  modified_base 45: mod_base = OTHER; note = Uracil
  modified_base 50: mod_base = OTHER; note = Uracil
  modified_base 52: mod_base = OTHER; note = Uracil
  modified_base 56: mod_base = OTHER; note = Uracil
  misc_feature 37..56: note = spacer sequence
cctgttgtgaatactcttttataggtatcaaacaacccgagtcctgcaatatgaat

SEQ ID NO: 169 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 39: mod_base = OTHER; note = Uracil
  modified_base 42: mod_base = OTHER; note = Uracil
  modified_base 47: mod_base = OTHER; note = Uracil
  modified_base 49: mod_base = OTHER; note = Uracil
  modified_base 53: mod_base = OTHER; note = Uracil
  modified_base 55: mod_base = OTHER; note = Uracil
  misc_feature 37..56: note = spacer sequence
cctgttgtgaatactcttttataggtatcaaacaacagtcctgcaatatgaatata

SEQ ID NO: 170 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 41: mod_base = OTHER; note = Uracil
  modified_base 46: mod_base = OTHER; note = Uracil
  modified_base 48: mod_base = OTHER; note = Uracil
  modified_base 52: mod_base = OTHER; note = Uracil
  modified_base 54: mod_base = OTHER; note = Uracil
  misc_feature 37..56: note = spacer sequence
cctgttgtgaatactcttttataggtatcaaacaacgtcctgcaatatgaatataa

SEQ ID NO: 171 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 40: mod_base = OTHER; note = Uracil
  modified_base 45: mod_base = OTHER; note = Uracil
  modified_base 47: mod_base = OTHER; note = Uracil
  modified_base 51: mod_base = OTHER; note = Uracil
  modified_base 53: mod_base = OTHER; note = Uracil
  modified_base 56: mod_base = OTHER; note = Uracil
  misc_feature 37..56: note = spacer sequence
cctgttgtgaatactcttttataggtatcaaacaactcctgcaatatgaatataat

SEQ ID NO: 172 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 39: mod_base = OTHER; note = Uracil
  modified_base 43: mod_base = OTHER; note = Uracil
  modified_base 45: mod_base = OTHER; note = Uracil
  modified_base 48: mod_base = OTHER; note = Uracil
  modified_base 49: mod_base = OTHER; note = Uracil
  modified_base 50: mod_base = OTHER; note = Uracil
  modified_base 51: mod_base = OTHER; note = Uracil
  misc_feature 37..56: note = spacer sequence
cctgttgtgaatactcttttataggtatcaaacaactatgaatataattttaaaaa

SEQ ID NO: 173 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 40: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  modified_base 52: mod_base = OTHER; note = Uracil
  misc_feature 37..56: note = spacer sequence
cctgttgtgaatactcttttataggtatcaaacaacatataattttaaaaatccaa

SEQ ID NO: 174 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 39: mod_base = OTHER; note = Uracil
  modified_base 42: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 51: mod_base = OTHER; note = Uracil
  modified_base 56: mod_base = OTHER; note = Uracil
  misc_feature 37..56: note = spacer sequence
cctgttgtgaatactcttttataggtatcaaacaactataattttaaaaatccaat

SEQ ID NO: 175 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 40: mod_base = OTHER; note = Uracil
  modified_base 41: mod_base = OTHER; note = Uracil
  modified_base 42: mod_base = OTHER; note = Uracil
  modified_base 43: mod_base = OTHER; note = Uracil
  modified_base 49: mod_base = OTHER; note = Uracil
  modified_base 54: mod_base = OTHER; note = Uracil
  modified_base 55: mod_base = OTHER; note = Uracil
  misc_feature 37..56: note = spacer sequence
cctgttgtgaatactcttttataggtatcaaacaactaattttaaaaatccaatta

SEQ ID NO: 176 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 41: mod_base = OTHER; note = Uracil
  modified_base 47: mod_base = OTHER; note = Uracil
  modified_base 52: mod_base = OTHER; note = Uracil
  modified_base 53: mod_base = OTHER; note = Uracil
  misc_feature 37..56: note = spacer sequence
cctgttgtgaatactcttttataggtatcaaacaacattttaaaaatccaattaag

SEQ ID NO: 177 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 46: mod_base = OTHER; note = Uracil
  modified_base 51: mod_base = OTHER; note = Uracil
  modified_base 52: mod_base = OTHER; note = Uracil
  misc_feature 37..56: note = spacer sequence
cctgttgtgaatactcttttataggtatcaaacaacttttaaaaatccaattaaga

SEQ ID NO: 178 - RNA, 56 nt, Synthetic construct; identical to SEQ ID NO: 125
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 41: mod_base = OTHER; note = Uracil
  modified_base 46: mod_base = OTHER; note = Uracil
  modified_base 47: mod_base = OTHER; note = Uracil
  misc_feature 37..56: note = spacer sequence

SEQ ID NO: 179 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 40: mod_base = OTHER; note = Uracil
  modified_base 45: mod_base = OTHER; note = Uracil
  modified_base 46: mod_base = OTHER; note = Uracil
  modified_base 56: mod_base = OTHER; note = Uracil
  misc_feature 37..56: note = spacer sequence
cctgttgtgaatactcttttataggtatcaaacaacaaatccaattaagagagagt

SEQ ID NO: 180 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 39: mod_base = OTHER; note = Uracil
  modified_base 44: mod_base = OTHER; note = Uracil
  modified_base 45: mod_base = OTHER; note = Uracil
  modified_base 55: mod_base = OTHER; note = Uracil
  misc_feature 37..56: note = spacer sequence
cctgttgtgaatactcttttataggtatcaaacaacaatccaattaagagagagtg

SEQ ID NO: 181 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 38: mod_base = OTHER; note = Uracil
  modified_base 43: mod_base = OTHER; note = Uracil
  modified_base 44: mod_base = OTHER; note = Uracil
  modified_base 54: mod_base = OTHER; note = Uracil
  misc_feature 37..56: note = spacer sequence
cctgttgtgaatactcttttataggtatcaaacaacatccaattaagagagagtga

SEQ ID NO: 182 - RNA, 56 nt, Synthetic construct
  misc_feature 1..56: note = synthetic Polynucleotide
  source 1..56: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 37: mod_base = OTHER; note = Uracil
  modified_base 42: mod_base = OTHER; note = Uracil
  modified_base 43: mod_base = OTHER; note = Uracil
  modified_base 53: mod_base = OTHER; note = Uracil
  modified_base 56: mod_base = OTHER; note = Uracil
  misc_feature 37..56: note = spacer sequence
cctgttgtgaatactcttttataggtatcaaacaactccaattaagagagagtgat

SEQ ID NO: 183 - RNA, 36 nt, Synthetic construct
  misc_feature 1..36: note = synthetic Polynucleotide
  source 1..36: mol_type = other RNA
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
cctgttgtgaatactcttttataggtatcaaacaac

SEQ ID NO: 184 - DNA, 20 bp, Synthetic construct
  misc_feature 1..20: note = synthetic Polynucleotide
  source 1..20: mol_type = other DNA
tgccccatcactctctctta

SEQ ID NO: 185 - DNA, 20 bp, Synthetic construct
  misc_feature 1..20: note = synthetic Polynucleotide
  source 1..20: mol_type = other DNA
ccccatcactctctcttaat

SEQ ID NO: 186 - DNA, 20 bp, Synthetic construct
  misc_feature 1..20: note = synthetic Polynucleotide
  source 1..20: mol_type = other DNA
tctcttaattggatttttaa

SEQ ID NO: 187 - DNA, 20 bp, Synthetic construct
  misc_feature 1..20: note = synthetic Polynucleotide
  source 1..20: mol_type = other DNA
tcttaattggatttttaaaa

SEQ ID NO: 188 - DNA, 20 bp, Synthetic construct
  misc_feature 1..20: note = synthetic Polynucleotide
  source 1..20: mol_type = other DNA
attggatttttaaaattata

SEQ ID NO: 189 - DNA, 20 bp, Synthetic construct
  misc_feature 1..20: note = synthetic Polynucleotide
  source 1..20: mol_type = other DNA
ttaaaattatattcatattg

SEQ ID NO: 190 - DNA, 20 bp, Synthetic construct
  misc_feature 1..20: note = synthetic Polynucleotide
  source 1..20: mol_type = other DNA
taaaattatattcatattgc

SEQ ID NO: 191 - DNA, 20 bp, Synthetic construct
  misc_feature 1..20: note = synthetic Polynucleotide
  source 1..20: mol_type = other DNA
aaaattatattcatattgca

SEQ ID NO: 192 - DNA, 20 bp, Synthetic construct
  misc_feature 1..20: note = synthetic Polynucleotide
  source 1..20: mol_type = other DNA
aaattatattcatattgcag

SEQ ID NO: 193 - DNA, 20 bp, Synthetic construct
  misc_feature 1..20: note = synthetic Polynucleotide
  source 1..20: mol_type = other DNA
tattcatattgcaggactcg

SEQ ID NO: 194 - DNA, 20 bp, Synthetic construct
  misc_feature 1..20: note = synthetic Polynucleotide
  source 1..20: mol_type = other DNA
atattgcaggactcggcaga

SEQ ID NO: 195 - DNA, 20 bp, Synthetic construct
  misc_feature 1..20: note = synthetic Polynucleotide
  source 1..20: mol_type = other DNA
caggactcggcagaagacct

SEQ ID NO: 196 - DNA, 20 bp, Synthetic construct
  misc_feature 1..20: note = synthetic Polynucleotide
  source 1..20: mol_type = other DNA
ggcagaagaccttcgagaga

SEQ ID NO: 197 - DNA, 20 bp, Synthetic construct
  misc_feature 1..20: note = synthetic Polynucleotide
  source 1..20: mol_type = other DNA
cgagagaaaggtagaaaata

SEQ ID NO: 198 - DNA, 20 bp, Synthetic construct
  misc_feature 1..20: note = synthetic Polynucleotide
  source 1..20: mol_type = other DNA
gagagaaaggtagaaaataa

SEQ ID NO: 199 - DNA, 20 bp, Synthetic construct
  misc_feature 1..20: note = synthetic Polynucleotide
  source 1..20: mol_type = other DNA
ttattttctacctttctctc

SEQ ID NO: 200 - DNA, 20 bp, Synthetic construct
  misc_feature 1..20: note = synthetic Polynucleotide
  source 1..20: mol_type = other DNA
attttctacctttctctcga

SEQ ID NO: 201 - DNA, 20 bp, Synthetic construct
  misc_feature 1..20: note = synthetic Polynucleotide
  source 1..20: mol_type = other DNA
ttttctacctttctctcgaa

SEQ ID NO: 202 - DNA, 20 bp, Synthetic construct
  misc_feature 1..20: note = synthetic Polynucleotide
  source 1..20: mol_type = other DNA
tctacctttctctcgaaggt

SEQ ID NO: 203 - DNA, 20 bp, Synthetic construct
  misc_feature 1..20: note = synthetic Polynucleotide
  source 1..20: mol_type = other DNA
ctacctttctctcgaaggtc

SEQ ID NO: 204 - DNA, 20 bp, Synthetic construct
  misc_feature 1..20: note = synthetic Polynucleotide
  source 1..20: mol_type = other DNA
tacctttctctcgaaggtct

SEQ ID NO: 205 - DNA, 20 bp, Synthetic construct
  misc_feature 1..20: note = synthetic Polynucleotide
  source 1..20: mol_type = other DNA
cctttctctcgaaggtcttc

SEQ ID NO: 206 - DNA, 20 bp, Synthetic construct
  misc_feature 1..20: note = synthetic Polynucleotide
  source 1..20: mol_type = other DNA
tctctcgaaggtcttctgcc

SEQ ID NO: 207 - DNA, 20 bp, Synthetic construct
  misc_feature 1..20: note = synthetic Polynucleotide
  source 1..20: mol_type = other DNA
ctctcgaaggtcttctgccg

SEQ ID NO: 208 - DNA, 20 bp, Synthetic construct
  misc_feature 1..20: note = synthetic Polynucleotide
  source 1..20: mol_type = other DNA
tctcgaaggtcttctgccga

SEQ ID NO: 209 - DNA, 20 bp, Synthetic construct
  misc_feature 1..20: note = synthetic Polynucleotide
  source 1..20: mol_type = other DNA
tcgaaggtcttctgccgagt

SEQ ID NO: 210 - DNA, 20 bp, Synthetic construct
  misc_feature 1..20: note = synthetic Polynucleotide
  source 1..20: mol_type = other DNA
gaaggtcttctgccgagtcc

SEQ ID NO: 211 - DNA, 20 bp, Synthetic construct
  misc_feature 1..20: note = synthetic Polynucleotide
  source 1..20: mol_type = other DNA
ctgccgagtcctgcaatatg

SEQ ID NO: 212 - DNA, 20 bp, Synthetic construct
  misc_feature 1..20: note = synthetic Polynucleotide
  source 1..20: mol_type = other DNA
tgccgagtcctgcaatatga

SEQ ID NO: 213 - DNA, 20 bp, Synthetic construct
  misc_feature 1..20: note = synthetic Polynucleotide
  source 1..20: mol_type = other DNA
ccgagtcctgcaatatgaat

SEQ ID NO: 214 - DNA, 20 bp, Synthetic construct
  misc_feature 1..20: note = synthetic Polynucleotide
  source 1..20: mol_type = other DNA
caatatgaatataattttaa

SEQ ID NO: 215 - DNA, 20 bp, Synthetic construct
  misc_feature 1..20: note = synthetic Polynucleotide
  source 1..20: mol_type = other DNA
aatataattttaaaaatcca

SEQ ID NO: 216 - DNA, 20 bp, Synthetic construct
  misc_feature 1..20: note = synthetic Polynucleotide
  source 1..20: mol_type = other DNA
attttaaaaatccaattaag

SEQ ID NO: 217 - DNA, 20 bp, Synthetic construct
  misc_feature 1..20: note = synthetic Polynucleotide
  source 1..20: mol_type = other DNA
taaaaatccaattaagagag

SEQ ID NO: 218 - DNA, 20 bp, Synthetic construct
  misc_feature 1..20: note = synthetic Polynucleotide
  source 1..20: mol_type = other DNA
aaaaatccaattaagagaga

SEQ ID NO: 219 - DNA, 20 bp, Synthetic construct
  misc_feature 1..20: note = synthetic Polynucleotide
  source 1..20: mol_type = other DNA
aaaatccaattaagagagag

SEQ ID NO: 220 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 38: mod_base = OTHER; note = Uracil
  modified_base 42: mod_base = OTHER; note = Uracil
  modified_base 44: mod_base = OTHER; note = Uracil
  modified_base 46: mod_base = OTHER; note = Uracil
  modified_base 48: mod_base = OTHER; note = Uracil
  modified_base 49: mod_base = OTHER; note = Uracil
  misc_feature 31..50: note = spacer sequence
ggtcccatcggaacgggttgtggttccgactgccccatcactctctctta

SEQ ID NO: 221 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 36: mod_base = OTHER; note = Uracil
  modified_base 40: mod_base = OTHER; note = Uracil
  modified_base 42: mod_base = OTHER; note = Uracil
  modified_base 44: mod_base = OTHER; note = Uracil
  modified_base 46: mod_base = OTHER; note = Uracil
  modified_base 47: mod_base = OTHER; note = Uracil
  modified_base 50: mod_base = OTHER; note = Uracil
  misc_feature 31..50: note = spacer sequence
ggtcccatcggaacgggttgtggttccgacccccatcactctctcttaat

SEQ ID NO: 222 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 33: mod_base = OTHER; note = Uracil
  modified_base 35: mod_base = OTHER; note = Uracil
  modified_base 36: mod_base = OTHER; note = Uracil
  modified_base 39: mod_base = OTHER; note = Uracil
  modified_base 40: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  modified_base 47: mod_base = OTHER; note = Uracil
  modified_base 48: mod_base = OTHER; note = Uracil
  misc_feature 31..50: note = spacer sequence
ggtcccatcggaacgggttgtggttccgactctcttaattggatttttaa

SEQ ID NO: 223 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 33: mod_base = OTHER; note = Uracil
  modified_base 34: mod_base = OTHER; note = Uracil
  modified_base 37: mod_base = OTHER; note = Uracil
  modified_base 38: mod_base = OTHER; note = Uracil
  modified_base 42: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 46: mod_base = OTHER; note = Uracil
  misc_feature 31..50: note = spacer sequence
ggtcccatcggaacgggttgtggttccgactcttaattggatttttaaaa

SEQ ID NO: 224 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 32: mod_base = OTHER; note = Uracil
  modified_base 33: mod_base = OTHER; note = Uracil
  modified_base 37: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 41: mod_base = OTHER; note = Uracil
  modified_base 46: mod_base = OTHER; note = Uracil
  modified_base 47: mod_base = OTHER; note = Uracil
  modified_base 49: mod_base = OTHER; note = Uracil
  misc_feature 31..50
ggtcccatcggaacgggttgtggttccgacattggatttttaaaattata

SEQ ID NO: 225 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 32: mod_base = OTHER; note = Uracil
  modified_base 37: mod_base = OTHER; note = Uracil
  modified_base 38: mod_base = OTHER; note = Uracil
  modified_base 40: mod_base = OTHER; note = Uracil
  modified_base 42: mod_base = OTHER; note = Uracil
  modified_base 43: mod_base = OTHER; note = Uracil
  modified_base 46: mod_base = OTHER; note = Uracil
  modified_base 48: mod_base = OTHER; note = Uracil
  modified_base 49: mod_base = OTHER; note = Uracil
  misc_feature 31..50
ggtcccatcggaacgggttgtggttccgacttaaaattatattcatattg

SEQ ID NO: 226 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 36: mod_base = OTHER; note = Uracil
  modified_base 37: mod_base = OTHER; note = Uracil
  modified_base 39: mod_base = OTHER; note = Uracil
  modified_base 41: mod_base = OTHER; note = Uracil
  modified_base 42: mod_base = OTHER; note = Uracil
  modified_base 45: mod_base = OTHER; note = Uracil
  modified_base 47: mod_base = OTHER; note = Uracil
  modified_base 48: mod_base = OTHER; note = Uracil
  misc_feature 31..50
ggtcccatcggaacgggttgtggttccgactaaaattatattcatattgc

SEQ ID NO: 227 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 35: mod_base = OTHER; note = Uracil
  modified_base 36: mod_base = OTHER; note = Uracil
  modified_base 38: mod_base = OTHER; note = Uracil
  modified_base 40: mod_base = OTHER; note = Uracil
  modified_base 41: mod_base = OTHER; note = Uracil
  modified_base 44: mod_base = OTHER; note = Uracil
  modified_base 46: mod_base = OTHER; note = Uracil
  modified_base 47: mod_base = OTHER; note = Uracil
  misc_feature 31..50
ggtcccatcggaacgggttgtggttccgacaaaattatattcatattgca

SEQ ID NO: 228 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 34: mod_base = OTHER; note = Uracil
  modified_base 35: mod_base = OTHER; note = Uracil
  modified_base 37: mod_base = OTHER; note = Uracil
  modified_base 39: mod_base = OTHER; note = Uracil
  modified_base 40: mod_base = OTHER; note = Uracil
  modified_base 43: mod_base = OTHER; note = Uracil
  modified_base 45: mod_base = OTHER; note = Uracil
  modified_base 46: mod_base = OTHER; note = Uracil
  misc_feature 31..50
ggtcccatcggaacgggttgtggttccgacaaattatattcatattgcag

SEQ ID NO: 229 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 33: mod_base = OTHER; note = Uracil
  modified_base 34: mod_base = OTHER; note = Uracil
  modified_base 37: mod_base = OTHER; note = Uracil
  modified_base 39: mod_base = OTHER; note = Uracil
  modified_base 40: mod_base = OTHER; note = Uracil
  modified_base 48: mod_base = OTHER; note = Uracil
  misc_feature 31..50
ggtcccatcggaacgggttgtggttccgactattcatattgcaggactcg

SEQ ID NO: 230 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 32: mod_base = OTHER; note = Uracil
  modified_base 34: mod_base = OTHER; note = Uracil
  modified_base 35: mod_base = OTHER; note = Uracil
  modified_base 43: mod_base = OTHER; note = Uracil
  misc_feature 31..50
ggtcccatcggaacgggttgtggttccgacatattgcaggactcggcaga

SEQ ID NO: 231 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 37: mod_base = OTHER; note = Uracil
  modified_base 50: mod_base = OTHER; note = Uracil
  misc_feature 30..50: note = spacer sequence
ggtcccatcggaacgggttgtggttccgaccaggactcggcagaagacct

SEQ ID NO: 232 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 42: mod_base = OTHER; note = Uracil
  modified_base 43: mod_base = OTHER; note = Uracil
  misc_feature 31..50
ggtcccatcggaacgggttgtggttccgacggcagaagaccttcgagaga

SEQ ID NO: 233 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 42: mod_base = OTHER; note = Uracil
  modified_base 49: mod_base = OTHER; note = Uracil
  misc_feature 31..50
ggtcccatcggaacgggttgtggttccgaccgagagaaaggtagaaaata

SEQ ID NO: 234 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 41: mod_base = OTHER; note = Uracil
  modified_base 48: mod_base = OTHER; note = Uracil
  misc_feature 31..50: note = spacer sequence
ggtcccatcggaacgggttgtggttccgacgagagaaaggtagaaaataa

SEQ ID NO: 235 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 32: mod_base = OTHER; note = Uracil
  modified_base 34: mod_base = OTHER; note = Uracil
  modified_base 35: mod_base = OTHER; note = Uracil
  modified_base 36: mod_base = OTHER; note = Uracil
  modified_base 37: mod_base = OTHER; note = Uracil
  modified_base 39: mod_base = OTHER; note = Uracil
  modified_base 43: mod_base = OTHER; note = Uracil
  modified_base 44: mod_base = OTHER; note = Uracil
  modified_base 45: mod_base = OTHER; note = Uracil
  modified_base 47: mod_base = OTHER; note = Uracil
  modified_base 49: mod_base = OTHER; note = Uracil
  misc_feature 31..50: note = spacer sequence
ggtcccatcggaacgggttgtggttccgacttattttctacctttctctc

SEQ ID NO: 236 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 32: mod_base = OTHER; note = Uracil
  modified_base 33: mod_base = OTHER; note = Uracil
  modified_base 34: mod_base = OTHER; note = Uracil
  modified_base 35: mod_base = OTHER; note = Uracil
  modified_base 37: mod_base = OTHER; note = Uracil
  modified_base 41: mod_base = OTHER; note = Uracil
  modified_base 42: mod_base = OTHER; note = Uracil
  modified_base 43: mod_base = OTHER; note = Uracil
  modified_base 45: mod_base = OTHER; note = Uracil
  modified_base 47: mod_base = OTHER; note = Uracil
  misc_feature 31..50: note = spacer sequence
ggtcccatcggaacgggttgtggttccgacattttctacctttctctcga

SEQ ID NO: 237 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 32: mod_base = OTHER; note = Uracil
  modified_base 33: mod_base = OTHER; note = Uracil
  modified_base 34: mod_base = OTHER; note = Uracil
  modified_base 36: mod_base = OTHER; note = Uracil
  modified_base 40: mod_base = OTHER; note = Uracil
  modified_base 41: mod_base = OTHER; note = Uracil
  modified_base 42: mod_base = OTHER; note = Uracil
  modified_base 44: mod_base = OTHER; note = Uracil
  modified_base 46: mod_base = OTHER; note = Uracil
  misc_feature 31..50: note = spacer sequence
ggtcccatcggaacgggttgtggttccgacttttctacctttctctcgaa

SEQ ID NO: 238 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 33: mod_base = OTHER; note = Uracil
  modified_base 37: mod_base = OTHER; note = Uracil
  modified_base 38: mod_base = OTHER; note = Uracil
  modified_base 39: mod_base = OTHER; note = Uracil
  modified_base 41: mod_base = OTHER; note = Uracil
  modified_base 43: mod_base = OTHER; note = Uracil
  modified_base 50: mod_base = OTHER; note = Uracil
  misc_feature 31..50: note = spacer sequence
ggtcccatcggaacgggttgtggttccgactctacctttctctcgaaggt

SEQ ID NO: 239 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 32: mod_base = OTHER; note = Uracil
  modified_base 36: mod_base = OTHER; note = Uracil
  modified_base 37: mod_base = OTHER; note = Uracil
  modified_base 38: mod_base = OTHER; note = Uracil
  modified_base 40: mod_base = OTHER; note = Uracil
  modified_base 42: mod_base = OTHER; note = Uracil
  modified_base 49: mod_base = OTHER; note = Uracil
  misc_feature 31..50: note = spacer sequence
ggtcccatcggaacgggttgtggttccgacctacctttctctcgaaggtc

SEQ ID NO: 240 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 35: mod_base = OTHER; note = Uracil
  modified_base 36: mod_base = OTHER; note = Uracil
  modified_base 37: mod_base = OTHER; note = Uracil
  modified_base 39: mod_base = OTHER; note = Uracil
  modified_base 41: mod_base = OTHER; note = Uracil
  modified_base 48: mod_base = OTHER; note = Uracil
  modified_base 50: mod_base = OTHER; note = Uracil
  misc_feature 31..50: note = spacer sequence
ggtcccatcggaacgggttgtggttccgactacctttctctcgaaggtct

SEQ ID NO: 241 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 33: mod_base = OTHER; note = Uracil
  modified_base 34: mod_base = OTHER; note = Uracil
  modified_base 35: mod_base = OTHER; note = Uracil
  modified_base 37: mod_base = OTHER; note = Uracil
  modified_base 39: mod_base = OTHER; note = Uracil
  modified_base 46: mod_base = OTHER; note = Uracil
  modified_base 48: mod_base = OTHER; note = Uracil
  modified_base 49: mod_base = OTHER; note = Uracil
  misc_feature 31..50: note = spacer sequence
ggtcccatcggaacgggttgtggttccgaccctttctctcgaaggtcttc

SEQ ID NO: 242 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 33: mod_base = OTHER; note = Uracil
  modified_base 35: mod_base = OTHER; note = Uracil
  modified_base 42: mod_base = OTHER; note = Uracil
  modified_base 44: mod_base = OTHER; note = Uracil
  modified_base 45: mod_base = OTHER; note = Uracil
  modified_base 47: mod_base = OTHER; note = Uracil
  misc_feature 31..50: note = spacer sequence
ggtcccatcggaacgggttgtggttccgactctctcgaaggtcttctgcc

SEQ ID NO: 243 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 32: mod_base = OTHER; note = Uracil
  modified_base 34: mod_base = OTHER; note = Uracil
  modified_base 41: mod_base = OTHER; note = Uracil
  modified_base 43: mod_base = OTHER; note = Uracil
  modified_base 44: mod_base = OTHER; note = Uracil
  modified_base 46: mod_base = OTHER; note = Uracil
  misc_feature 31..50: note = spacer sequence
ggtcccatcggaacgggttgtggttccgacctctcgaaggtcttctgccg

SEQ ID NO: 244 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 33: mod_base = OTHER; note = Uracil
  modified_base 40: mod_base = OTHER; note = Uracil
  modified_base 42: mod_base = OTHER; note = Uracil
  modified_base 43: mod_base = OTHER; note = Uracil
  modified_base 45: mod_base = OTHER; note = Uracil
  misc_feature 31..50: note = spacer sequence
ggtcccatcggaacgggttgtggttccgactctcgaaggtcttctgccga

SEQ ID NO: 245 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 38: mod_base = OTHER; note = Uracil
  modified_base 40: mod_base = OTHER; note = Uracil
  modified_base 41: mod_base = OTHER; note = Uracil
  modified_base 43: mod_base = OTHER; note = Uracil
  modified_base 50: mod_base = OTHER; note = Uracil
  misc_feature 31..50: note = spacer sequence
ggtcccatcggaacgggttgtggttccgactcgaaggtcttctgccgagt

SEQ ID NO: 246 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 36: mod_base = OTHER; note = Uracil
  modified_base 38: mod_base = OTHER; note = Uracil
  modified_base 39: mod_base = OTHER; note = Uracil
  modified_base 41: mod_base = OTHER; note = Uracil
  modified_base 48: mod_base = OTHER; note = Uracil
  misc_feature 31..50: note = spacer sequence
ggtcccatcggaacgggttgtggttccgacgaaggtcttctgccgagtcc

SEQ ID NO: 247 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 32: mod_base = OTHER; note = Uracil
  modified_base 39: mod_base = OTHER; note = Uracil
  modified_base 42: mod_base = OTHER; note = Uracil
  modified_base 47: mod_base = OTHER; note = Uracil
  modified_base 49: mod_base = OTHER; note = Uracil
  misc_feature 31..50: note = spacer sequence
ggtcccatcggaacgggttgtggttccgacctgccgagtcctgcaatatg

SEQ ID NO: 248 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 38: mod_base = OTHER; note = Uracil
  modified_base 41: mod_base = OTHER; note = Uracil
  modified_base 46: mod_base = OTHER; note = Uracil
  modified_base 48: mod_base = OTHER; note = Uracil
  misc_feature 31..50: note = spacer sequence
ggtcccatcggaacgggttgtggttccgactgccgagtcctgcaatatga

SEQ ID NO: 249 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 36: mod_base = OTHER; note = Uracil
  modified_base 39: mod_base = OTHER; note = Uracil
  modified_base 44: mod_base = OTHER; note = Uracil
  modified_base 46: mod_base = OTHER; note = Uracil
  modified_base 50: mod_base = OTHER; note = Uracil
  misc_feature 31..50: note = spacer sequence
ggtcccatcggaacgggttgtggttccgacccgagtcctgcaatatgaat

SEQ ID NO: 250 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 34: mod_base = OTHER; note = Uracil
  modified_base 36: mod_base = OTHER; note = Uracil
  modified_base 40: mod_base = OTHER; note = Uracil
  modified_base 42: mod_base = OTHER; note = Uracil
  modified_base 45: mod_base = OTHER; note = Uracil
  modified_base 46: mod_base = OTHER; note = Uracil
  modified_base 47: mod_base = OTHER; note = Uracil
  modified_base 48: mod_base = OTHER; note = Uracil
  misc_feature 31..50: note = spacer sequence
ggtcccatcggaacgggttgtggttccgaccaatatgaatataattttaa

SEQ ID NO: 251 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 33: mod_base = OTHER; note = Uracil
  modified_base 35: mod_base = OTHER; note = Uracil
  modified_base 38: mod_base = OTHER; note = Uracil
  modified_base 39: mod_base = OTHER; note = Uracil
  modified_base 40: mod_base = OTHER; note = Uracil
  modified_base 41: mod_base = OTHER; note = Uracil
  modified_base 47: mod_base = OTHER; note = Uracil
  misc_feature 31..50: note = spacer sequence
ggtcccatcggaacgggttgtggttccgacaatataattttaaaaatcca

SEQ ID NO: 252 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 32: mod_base = OTHER; note = Uracil
  modified_base 33: mod_base = OTHER; note = Uracil
  modified_base 34: mod_base = OTHER; note = Uracil
  modified_base 35: mod_base = OTHER; note = Uracil
  modified_base 41: mod_base = OTHER; note = Uracil
  modified_base 46: mod_base = OTHER; note = Uracil
  modified_base 47: mod_base = OTHER; note = Uracil
  misc_feature 31..50: note = spacer sequence
ggtcccatcggaacgggttgtggttccgacattttaaaaatccaattaag

SEQ ID NO: 253 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 31: mod_base = OTHER; note = Uracil
  modified_base 37: mod_base = OTHER; note = Uracil
  modified_base 42: mod_base = OTHER; note = Uracil
  modified_base 43: mod_base = OTHER; note = Uracil
  misc_feature 31..50: note = spacer sequence
ggtcccatcggaacgggttgtggttccgactaaaaatccaattaagagag

SEQ ID NO: 254 - RNA, 50 nt, Synthetic construct
  misc_feature 1..50: note = synthetic Polynucleotide
  source 1..50: mol_type = other RNA
  modified_base 3: mod_base = OTHER; note = Uracil
  modified_base 8: mod_base = OTHER; note = Uracil
  modified_base 18: mod_base = OTHER; note = Uracil
  modified_base 19: mod_base = OTHER; note = Uracil
  modified_base 21: mod_base = OTHER; note = Uracil
  modified_base 24: mod_base = OTHER; note = Uracil
  modified_base 25: mod_base = OTHER; note = Uracil
  modified_base 36: mod_base = OTHER; note = Uracil
  modified_base 41: mod_base = OTHER; note = Uracil
  modified_base 42: mod_base = OTHER; note = Uracil
  misc_feature 31..50: note = spacer sequence
ggtcccatcggaacgggttgtggttccgacaaaaatccaattaagagaga

SEQ ID NO: 255 - protein, 745 aa, Synthetic construct
  REGION 1..745: note = synthetic polypeptide
MIKSIQLKVKGECRITKDVINEYKEYYNNCSRWIKNNLTSITIGEMAKFLQSLSDKEVAYRSMGLSDEWKDKPLYHLFTKKYHTKNADNLLYYYIKEKNLDGYKGNTLNISNTSFRQFGYFKLVVSNYRTKIRTLNCKIKRKKIDADSTSEDIEMQVMYEIIKYSLNKKSDWDNFISYIENVENPNIDNINRYKLLRECFCENENMIKNKLELLSVEQLKKFGGCIMKPHINSMTINIQDFKIEEKENSLGFILHLPLNKKQYQIELLGNRQIKKGTKEIHETLVDITNTHGENIVFTIKNDNLYIVFSYRSEFEKEEVNFAKTVGLDVNFKHAFFVTSEKDNCHLDGYINLYKYLLEHDEFTNLLTEDERKDYEELSKVVTFCPFENQLLFARYNKMSKFCKKEQVLSKLLYALQKKLKDENRTKEYIYVSCVNKLRAKYVSYFILKEKYYEKQKEYDIEMGFVDDSTESKESMDKRRTEYPFRNTPVANELLSKLNNVQQDINGCLKNIINYIYKIFEQNGYKVVALENLENSNFEKKQVLPTIKSLLKYHKLENQNVNDIKASDKVKEYIENGYYELMTNENNEIVDAKYTEKGAMKVKNANFFNLMMKSLHFASVKDEFVLLSNNGKTQIALVPSEFTSQMDSTDHCLYMKKNDKGKLVKADKKEVRTKQERHINGLNADFNAANNIKYIVENEVWRGIFCTRPKKTEYNVPSLDTTKKGPSAILNMLKKIEAIKVLETEK

SEQ ID NO: 256 - RNA, 245 nt, Synthetic construct
  misc_feature 1..245: note = synthetic Polynucleotide
  source 1..245: mol_type = other RNA
  modified_base 1: mod_base = OTHER; note = Uracil
  modified_base 2: mod_base = OTHER; note = Uracil
  modified_base 10: mod_base = OTHER; note = Uracil
  modified_base 23: mod_base = OTHER; note = Uracil
  modified_base 24: mod_base = OTHER; note = Uracil
  modified_base 36: mod_base = OTHER; note = Uracil
  modified_base 43: mod_base = OTHER; note = Uracil
  modified_base 49: mod_base = OTHER; note = Uracil
  modified_base 50: mod_base = OTHER; note = Uracil
  modified_base 51: mod_base = OTHER; note = Uracil
  modified_base 55: mod_base = OTHER; note = Uracil
  modified_base 57: mod_base = OTHER; note = Uracil
  modified_base 59: mod_base = OTHER; note = Uracil
  modified_base 61: mod_base = OTHER; note = Uracil
  modified_base 63: mod_base = OTHER; note = Uracil
  modified_base 69: mod_base = OTHER; note = Uracil
  modified_base 71: mod_base = OTHER; note = Uracil
  modified_base 73: mod_base = OTHER; note = Uracil
  modified_base 77: mod_base = OTHER; note = Uracil
  modified_base 79: mod_base = OTHER; note = Uracil
  modified_base 81: mod_base = OTHER; note = Uracil
  modified_base 105: mod_base = OTHER; note = Uracil
  modified_base 109: mod_base = OTHER; note = Uracil
  modified_base 119: mod_base = OTHER; note = Uracil
  modified_base 123: mod_base = OTHER; note = Uracil
  modified_base 125: mod_base = OTHER; note = Uracil
  modified_base 129: mod_base = OTHER; note = Uracil
  modified_base 135: mod_base = OTHER; note = Uracil
  modified_base 136: mod_base = OTHER; note = Uracil
  modified_base 138: mod_base = OTHER; note = Uracil
  modified_base 146: mod_base = OTHER; note = Uracil
  modified_base 147: mod_base = OTHER; note = Uracil
  modified_base 156: mod_base = OTHER; note = Uracil
  modified_base 159: mod_base = OTHER; note = Uracil
  modified_base 163: mod_base = OTHER; note = Uracil
  modified_base 167: mod_base = OTHER; note = Uracil
  modified_base 170: mod_base = OTHER; note = Uracil
  modified_base 177: mod_base = OTHER; note = Uracil
  modified_base 178: mod_base = OTHER; note = Uracil
  modified_base 181: mod_base = OTHER; note = Uracil
  modified_base 183: mod_base = OTHER; note = Uracil
  modified_base 186: mod_base = OTHER; note = Uracil
  modified_base 187: mod_base = OTHER; note = Uracil
  modified_base 188: mod_base = OTHER; note = Uracil
  modified_base 190: mod_base = OTHER; note = Uracil
  modified_base 194: mod_base = OTHER; note = Uracil
  modified_base 200: mod_base = OTHER; note = Uracil
  modified_base 203: mod_base = OTHER; note = Uracil
  modified_base 211: mod_base = OTHER; note = Uracil
  modified_base 212: mod_base = OTHER; note = Uracil
  modified_base 224: mod_base = OTHER; note = Uracil
  modified_base 225: mod_base = OTHER; note = Uracil
  modified_base 228: mod_base = OTHER; note = Uracil
  modified_base 230: mod_base = OTHER; note = Uracil
  modified_base 232: mod_base = OTHER; note = Uracil
  intron 1..6: note = TTGCAG
  polyA_site 210..215: note = premature poly(a) site
  protein_bind 59..63: bound_moiety = TDP-43 binding motif
  protein_bind 69..74: bound_moiety = TDP-43 binding
  protein_bind 76..81: bound_moiety = TDP-43 binding motif
ttgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaaaaaaaaaaaa

SEQ ID NO: 257 - DNA, 21 bp, Synthetic construct
  misc_feature 1..21: note = synthetic Polynucleotide
  source 1..21: mol_type = other DNA
ctttctctagcacggtcccac

SEQ ID NO: 258 - DNA, 23 bp, Synthetic construct
  misc_feature 1..23: note = synthetic Polynucleotide
  source 1..23: mol_type = other DNA
atgctcacacagagagccaaatt

SEQ ID NO: 259 - DNA, 20 bp, Synthetic construct
  misc_feature 1..20: note = synthetic Polynucleotide
  source 1..20: mol_type = other DNA
ctctcgaaggtcttctgccg

SEQ ID NO: 260 - RNA, 56 nt, Synthetic construct
  misc_feature 1..56: note = synthetic Polynucleotide
  source 1..56: mol_type = other RNA
  modified_base 2: mod_base = OTHER; note = Uracil
  modified_base 3: mod_base = OTHER; note = Uracil
  modified_base 5: mod_base = OTHER; note = Uracil
  modified_base 6: mod_base = OTHER; note = Uracil
  modified_base 8: mod_base = OTHER; note = Uracil
  modified_base 10: mod_base = OTHER; note = Uracil
  modified_base 12: mod_base = OTHER; note = Uracil
  modified_base 14: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 20: mod_base = OTHER; note = Uracil
  modified_base 22: mod_base = OTHER; note = Uracil
  modified_base 26: mod_base = OTHER; note = Uracil
  modified_base 28: mod_base = OTHER; note = Uracil
  modified_base 29: mod_base = OTHER; note = Uracil
  modified_base 39: mod_base = OTHER; note = Uracil
  modified_base 43: mod_base = OTHER; note = Uracil
  modified_base 50: mod_base = OTHER; note = Uracil
cttgttgtatatgtccttttataggtattaaacaacagtgcgtacgagctcggacg

SEQ ID NO: 261 - DNA, 48 bp, Synthetic construct
  misc_feature 1..21: note = synthetic Polynucleotide
  source 1..48: mol_type = other DNA
agatcttaagactggtcattcctcgaggaatgaccagtcttaagatct

SEQ ID NO: 262 - DNA, 48 bp, Synthetic construct
  misc_feature 1..21: note = synthetic Polynucleotide
  source 1..48: mol_type = other DNA
cctaaggttaagtcgccctcgctcgagcgagggcgacttaaccttagg

SEQ ID NO: 263 - DNA, 22 bp, synthetic construct
  source 1..22: mol_type = other DNA
gccttactcagactcctctctc

SEQ ID NO: 264 - DNA, 19 bp, synthetic construct
  source 1..19: mol_type = other DNA
tcttctgccgagtcccatt

SEQ ID NO: 265 - DNA, 24 bp, synthetic construct
  source 1..24: mol_type = other DNA
ctggacccttctcctttgccttcg

SEQ ID NO: 266 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 267 - RNA, 20 nt, synthetic construct
  source 1..20: mol_type = other RNA
ctacctttctctcgaaggtc

SEQ ID NO: 268 - RNA, 20 nt, synthetic construct
  source 1..20: mol_type = other RNA
ctctcgaaggtcttctgccg

SEQ ID NO: 269 - RNA, 20 nt, synthetic construct
  source 1..20: mol_type = other RNA
tctcgaaggtcttctgccga

SEQ ID NO: 270 - RNA, 20 nt, synthetic construct
  source 1..20: mol_type = other RNA
tattcatattgcaggactcg

SEQ ID NO: 271 - RNA, 20 nt, synthetic construct
  source 1..20: mol_type = other RNA
aaattatattcatattgcag